(19) United States
(12) Patent Application Publication
(10) Pub. No.: US 2023/0340498 A1
(43) Pub. Date: Oct. 26, 2023

(54) COMPOSITIONS AND METHODS FOR INDUCIBLE ALTERNATIVE SPLICING REGULATION OF GENE EXPRESSION

(71) Applicant: The Children's Hospital of Philadelphia, Philadelphia, PA (US)

(72) Inventors: Beverly L. DAVIDSON, Philadelphia, PA (US); Alejandro Mas MONTEYS, Philadelphia, PA (US); Amiel A. HUNDLEY, Neptune, NJ (US); Paul T. RANUM, Gainesville, FL (US)

(73) Assignee: The Children's Hospital of Philadelphia, Philadelphia, PA (US)

(21) Appl. No.: 17/798,851

(22) PCT Filed: Feb. 12, 2021
(86) PCT No.: PCT/US2021/017950

§ 371 Date: Aug. 10, 2022

Related U.S. Application Data
(60) Provisional application No. 62/975,400, filed on Feb. 12, 2020.

(51) Int. Cl. C12N 15/62; A61K 48/00; C12N 15/86; C07K 14/015

(52) U.S. Cl. CPC C12N 15/625; A61K 48/005; C07K 14/015; C12N 15/86; C07K 2319/02; C12N 2320/33; C12N 2750/14143

(57) ABSTRACT

Provided herein are chimeric minigenes, where the alternative splicing of the minigene determines whether an encoded gene is expressed. In particular, the minigenes are alternatively spliced in response to splicing modulator dmgs, such that the encoded gene is only expressed in the present of the splicing modulator dmg. The encoded gene may encode an inhibitory RNA, a CRISPR-Cas9 protein, a transactivator, or a therapeutic protein.

### REFERENCE TO RELATED APPLICATIONS

The present application is a national phase application under 35 U.S.C. § 371 of International Application No. PCT/US2021/017950, filed Feb. 12, 2021, which claims the priority benefit of United States provisional application number 62/975,400, filed Feb. 12, 2020, the entire contents of each of which are incorporated herein by reference.

### REFERENCE TO A SEQUENCE LISTING

The instant application contains a Sequence Listing, which has been submitted in ASCII format via EFS-Web and is hereby incorporated by reference in its entirety. Said ASCII copy, created on Apr. 3, 2023, is named CHOPP0041US_ST25.txt and is 36,474 bytes in size.

### BACKGROUND

### 1. Field

The present invention relates generally to the fields of molecular biology and medicine. More particularly, it concerns compositions and methods for using alternative splicing regulation to modulate expression of a therapeutic gene.

### 2. Description of Related Art

While viral and nonviral approaches for gene therapies have made tremendous advancements over the last twenty years, the major focus has been on the cargo delivery system; e.g., viral capsid evolution and engineering for adeno-associated viruses (AAVs), expanding the landscape of cell-targeting envelopes for lentiviruses, and refining lipid nanoparticles for improved uptake. However, the cargo itself, and more importantly the elements controlling the expression from that cargo, have been largely untouched aside from using engineered promoters or 3′ regulatory elements to restrict expression to certain cell types (Brown et al., 2006; Domenger & Grimm, 2019). As such, compositions and methods for modulating expression of therapeutic genes in cargo delivery systems are needed.

### SUMMARY

Provided herein are compositions and methods for finely controlling gene expression via a drug inducible alternative splicing switch. Importantly, these compositions and methods do not require any bacterial or other external elements for regulation. These compositions and methods can be applied to any genetic element of interest in cells or animals, and take advantage of drugs that are orally bioavailable and in human use.

In one embodiment, provided herein are nucleic acid molecules comprising a first expression cassette comprising, from 5′ to 3′, (a) a minigene having an alternatively spliced exon and (b) an encoded gene. In some aspects, the alternatively spliced exon is a pseudoexon. In some aspects, the minigene comprises, from 5′ to 3′, Exon 1, Intron 1, Exon 2, Intron 2, and Exon 3, wherein Exon 2 is the alternatively spliced exon, and wherein Exon 2 comprises translation initiation regulatory sequences.

In some aspects, inclusion of Exon 2 causes a frameshift. In some aspects, the number of nucleotides present in Exon 2 is not divisible by 3. In some aspects, Exon 3 comprises a stop codon that is in frame when Exon 2 is skipped. In some aspects, the encoded gene is in frame with the translation initiation regulatory sequence in Exon 2.

In some aspects, the encoded gene encodes a signal peptide such that the encoded protein enters the secretory pathway. In some aspects, the amino acids encoded by Exon 2 of the minigene correspond to a sequence of a predicted signal peptide. The sequence of the predicted signal peptide may correspond to the native signal peptide of the encoded gene or to a signal peptide that is heterologous to the encoded gene. In some aspects, at least a portion of the native signal peptide of the encoded gene is deleted, such that the protein produced has a signal peptide that is partially encoded by Exons 2 and 3 of the minigene and partially encoded by the encoded gene.

In some aspects, Exon 2 comprises a sequence according to nucleotides 1203-1257 of SEQ ID NO: 1. In some aspects, Intron 1 comprises a sequence according to nucleotides 159-1202 of SEQ ID NO: 1, or a fragment thereof having at least 90%, at least 95%, at least 96%, at least 97%, at least 98%, or at least 99% identity thereto. In some aspects, Intron 2 comprises a sequence according to nucleotides 1258-1701 of SEQ ID NO: 1, or a fragment thereof having at least 90%, at least 95%, at least 96%, at least 97%, at least 98%, or at least 99% identity thereto. In some aspects, the minigene comprises a sequence according to SEQ ID NO: 1.

In some aspects, Exon 2 comprises a sequence according to nucleotides 595-653 of SEQ ID NO: 4, or a fragment thereof having at least 90%, at least 95%, at least 96%, at least 97%, at least 98%, or at least 99% identity thereto. In some aspects, Intron 1 comprises a sequence according to nucleotides 97-594 of SEQ ID NO: 4, or a fragment thereof having at least 90%, at least 95%, at least 96%, at least 97%, at least 98%, or at least 99% identity thereto. In some aspects, Intron 2 comprises a sequence according to nucleotides 654-1153 of SEQ ID NO: 4, or a fragment thereof having at least 90%, at least 95%, at least 96%, at least 97%, at least 98%, or at least 99% identity thereto. In some aspects, the minigene comprises a sequence according to SEQ ID NO: 4.

In some aspects, Exon 2 comprises a sequence according to nucleotides 427-471 of SEQ ID NO: 10, or a fragment thereof having at least 90%, at least 95%, at least 96%, at least 97%, at least 98%, or at least 99% identity thereto. In some aspects, Intron 1 comprises a sequence according to nucleotides 103-426 of SEQ ID NO: 10, or a fragment thereof having at least 90%, at least 95%, at least 96%, at least 97%, at least 98%, or at least 99% identity thereto. In some aspects, Intron 2 comprises a sequence according to nucleotides 472-834 of SEQ ID NO: 10, or a fragment thereof having at least 90%, at least 95%, at least 96%, at least 97%, at least 98%, or at least 99% identity thereto. In some aspects, the minigene comprises a sequence according to SEQ ID NO: 10.

In some aspects, Exon 2 comprises a sequence according to nucleotides 621-759 of SEQ ID NO: 11, or a fragment thereof having at least 90%, at least 95%, at least 96%, at least 97%, at least 98%, or at least 99% identity thereto. In some aspects, Intron 1 comprises a sequence according to nucleotides 119-620 of SEQ ID NO: 11, or a fragment thereof having at least 90%, at least 95%, at least 96%, at least 97%, at least 98%, or at least 99% identity thereto. In some aspects, Intron 2 comprises a sequence according to nucleotides 760-1228 of SEQ ID NO: 11, or a fragment thereof having at least 90%, at least 95%, at least 96%, at least 97%, at least 98%, or at least 99% identity thereto. In some aspects, the minigene comprises a sequence according to SEQ ID NO: 11.

In some aspects, Exon 2 comprises a sequence according to nucleotides 750-817 of SEQ ID NO: 12, or a fragment thereof having at least 90%, at least 95%, at least 96%, at least 97%, at least 98%, or at least 99% identity thereto. In some aspects, Intron 1 comprises a sequence according to nucleotides 99-749 of SEQ ID NO: 12. In some aspects, Intron 2 comprises a sequence according to nucleotides 818-936 of SEQ ID NO: 12, or a fragment thereof having at least 90%, at least 95%, at least 96%, at least 97%, at least 98%, or at least 99% identity thereto. In some aspects, the minigene comprises a sequence according to SEQ ID NO: 12.

In some aspects, Exon 2 comprises a sequence according to nucleotides 593-650 of SEQ ID NO: 13, or a fragment thereof having at least 90%, at least 95%, at least 96%, at least 97%, at least 98%, or at least 99% identity thereto. In some aspects, Exon 3 comprises a sequence according to nucleotides 1149-1153 of any of SEQ ID NOs: 13-15, or a fragment thereof having at least 90%, at least 95%, at least 96%, at least 97%, at least 98%, or at least 99% identity thereto. In some aspects, Intron 1 comprises a sequence according to nucleotides 96-592 of SEQ ID NO: 13, or a fragment thereof having at least 90%, at least 95%, at least 96%, at least 97%, at least 98%, or at least 99% identity thereto. In some aspects, Intron 2 comprises a sequence according to nucleotides 651-1148 of SEQ ID NO: 13, or a fragment thereof having at least 90%, at least 95%, at least 96%, at least 97%, at least 98%, or at least 99% identity thereto. In some aspects, the minigene comprises a sequence according to any of SEQ ID NOs: 13-15.

In some aspects, the minigene comprises fewer than 2000, fewer than 1900, fewer than 1800, fewer than 1700, fewer than 1600, fewer than 1500, fewer than 1400, fewer than 1300, fewer than 1200, fewer than 1100, fewer than 1000, fewer than 900, fewer than 800, fewer than 700, fewer than 600 or fewer than 500 nucleotides.

In some aspects, the expression of the encoded gene does not require the co-expression of any exogenous regulatory protein. In some aspects, the encoded gene encodes an inhibitory RNA, a therapeutic protein, a Cas9 protein, or a transactivator protein. In some aspects, the inhibitory RNA is a siRNA, shRNA, or miRNA. In some aspects, the inhibitory RNA inhibits or decreases expression of an aberrant or abnormal protein associated with a disease. In some aspects, the therapeutic protein is a protein whose deficiency is associated with a disease. In some aspects, the encoded is not a reporter.

In some aspects, the minigene and the encoded gene are separated by a cleavable peptide.

In some aspects, the first expression cassette is operably linked to a first promoter. In some aspects, the first promoter is a constitutive promoter. In some aspects, the first promoter is a Rous sarcoma virus (RSV) promoter, the phosphoglycerate kinase (PGK) promoter, a JeT promoter, a CBA promoter, a synapsin promoter, or the minimal cytomegalovirus (mCMV) promoter.

In some aspects, the nucleic acid molecules further comprise a second expression cassette. In some aspects, the second expression cassette comprises a nucleic acid sequence encoding a guide RNA operably linked to a second promoter. In some aspects, the second expression cassette comprises a nucleic acid sequence encoding a therapeutic protein, an inhibitory RNA, or a Cas9 protein, wherein the nucleic acid sequence is operably linked to a second promoter, wherein the second promoter is activated by the transactivator encoded by the first expression cassette.

In one embodiment, provided herein are cells comprising the nucleic acid molecule of any one of the present embodiments.

In one embodiment, provided herein are recombinant adeno-associated virus (rAAV) vectors comprising an AAV capsid protein and nucleic acid molecule of any one of the present embodiments.

In one embodiment, provided herein are methods of inducing the expression of the encoded gene in a cell any one of the present embodiments, the methods comprising contacting the cell with a splicing modifier drug. In some aspects, in the presence of the splice modifier drug, the second exon is included in an mRNA product of the nucleic acid, and in the absent of said splice modifier drug, said exon is not included in an mRNA product of the nucleic acid. In some aspects, the splicing modifier drug is LMI070 or RG7800/RG7619.

In one embodiment, provided herein are methods of administering the encoded gene to a patient in need thereof, the method comprising administering the nucleic acid molecule of any one of the present embodiments to the patient. In some aspects, administering the encoded gene comprises administering an rAAV of any one of the present embodiments to the patient.

In some aspects, the expression of the encoded gene is regulated by a disease state in the patient. In some aspects, Exon 2 is only included in a diseased cell, such that the encoded gene is only expressed in the diseased cell. For example, the minigene may comprise PCDH1 (5 : 141869432 - 141878222) such that the encoded gene is only expressed in a cell expressing mutant HTT.

In some aspects, the expression of the encoded gene is regulated by a cell type or tissue type. In some aspects, Exon 2 is only included in the cell type or tissue type.

In some aspects, the methods further comprise administering a splicing modifier drug to the patient to induce expression of the encoded gene. In some aspects, the splicing modifier drug is LMI070 or RG7800/RG7619. In some aspects, administering the splicing modifier drug is performed more than once. In some aspects, administering the splicing modifier drug is performed at regular intervals. In some aspects, administering the splicing modifier drug causes increase in expression of the encoded gene, for example, by at least 2, 3, 4, 5, 6, 7, 8, 9, 10, 20, 30, 50 or 100 fold. In some aspects, administering the splicing modifier drug causes at least a 20-fold increase in expression of the encoded gene.

In some aspects, the rAAV vector comprises an AAV particle comprising AAV capsid proteins, and wherein the first and/or second expression cassette is inserted between a pair of AAV inverted terminal repeats (ITRs). In some aspects, the rAAV is a self-complementary AAV (scAAV) vector. In some aspects, the rAAV is a single-stranded AAV (ssAAV). In some aspects, the AAV capsid proteins are derived from or selected from the group consisting of AAV1, AAV2, AAV3, AAV4, AAV5, AAV6, AAV7, AAV8, AAV9, AAV10, AAV11, AAV12, AAV-rh74, AAV-rh10, and AAV-2i8 VP1, VP2 and/or VP3 capsid proteins, or a capsid protein having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 VP1, VP2 and/or VP3 capsid proteins. In some aspects, the pair of AAV ITRs is derived from, comprises or consists of an AAV1, AAV2, AAV3, AAV4, AAV5, AAV6, AAV7, AAV8, AAV9, AAV10, AAV11, AAV12, AAV-rh74, AAV-rh10 or AAV-2i8 ITR, or an ITR having 70% or more identity to AAV1, AAV2, AAV3, AAV4, AAV5, AAV6, AAV7, AAV8, AAV9, AAV10, AAV11, AAV12, AAV-rh74, AAV-Rh10, or AAV-2i8 ITR sequence.

In some aspects, a plurality of the viral vectors are administered. In some aspects, the viral vectors are administered at a dose of about 1×10⁶ to about 1×10¹⁸ vector genomes per kilogram (vg/kg). In some aspects, the viral vectors are administered at a dose from about 1x10⁷-1x10¹⁷, about 1x10⁸-1x10¹⁶, about 1x10⁹-1x10¹⁵, about 1x10¹⁰-1x10¹⁴, about 1x10¹⁰-1x10¹³, about 1x10¹⁰-1x10¹³, about 1x10¹⁰-1x10¹¹, about 1x10¹¹-1x10¹², about 1x10¹²-x10¹³, or about 1x10¹³-1X10¹⁴ vg/kg of the patient. In some aspects, the viral vectors are administered at a dose of about 0.5-4 ml of 1x10⁶ -1x10¹⁶ vg/ml.

In some aspects, the methods further comprise administering a plurality of empty viral capsids. In some aspects, the empty viral capsids are formulated with the viral particles administered to the patient. In some aspects, the empty viral capsids are administered or formulated with 1.0 to 100-fold excess of viral vector particles or empty viral capsids. In some aspects, the empty viral capsids are administered or formulated with 1.0 to 100-fold excess of viral vector particles to empty viral capsids. In some aspects, the empty viral capsids are administered or formulated with about 1.0 to 100-fold excess of empty viral capsids to viral vector particles.

In some aspects, the administration is to the central nervous system. In some aspects, the administration is to the brain. In some aspects, the administration is to a cisterna magna, an intraventricular space, an ependyma, a brain ventricle, a subarachnoid space, and/or an intrathecal space. In some aspects, the brain ventricle is the rostral lateral ventricle, and/or the caudal lateral ventricle, and/or the right lateral ventricle, and/or the left lateral ventricle, and/or the right rostral lateral ventricle, and/or the left rostral lateral ventricle, and/or the right caudal lateral ventricle, and/or the left caudal lateral ventricle. In some aspects, the administering comprises intraventricular injection and/or intraparenchymal injection. In some aspects, the administration is at a single location in the brain. In some aspects, the administration is at 1-5 locations in the brain.

In some aspects, the patient is a human.

In some aspects, the methods further comprise administering one or more immunosuppressive agents. In some aspects, the immunosuppressive agent is administered prior to or contemporaneously with administration of the expression cassettes. In some aspects, the immunosuppressive agent is an anti-inflammatory agent.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, the variation that exists among the study subjects, or a value that is within 10% of a stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1H. Generation and assessment of the SMN2-on cassette. (FIG. 1A) Cartoon depicting the SMN2-on cassette and its mechanism of action. In the absence of exon 7 (e7), a premature stop codon in the exon 6/8 transcript (e6/8) blocks translation of the luciferase cDNA sequence, while inclusion of the alternative splice exon 7 by LMI070 or RG7800 (depicted by arrow) permits translation of the e6/7/8:Firefly luciferase fusion protein. (FIG. 1B) Cartoon depicting SMN2 exon 7 in its native sequence (see positions 1196-1206 and 1254-1262 of SEQ ID NO: 1), or with the splice site modifications introduced for constitutive inclusion (5′ donor splice site, actSMN2) (see positions 1196-1206 and 1254-1262 of SEQ ID NO: 3) or for reduced background levels of exon 7 inclusion (3′ acceptor splice site, indSMN2) (see positions 1196-1206 and 1254-1262 of SEQ ID NO: 2). (FIG. 1C) Representative RT-PCR reaction showing exon 7 inclusion with the SMN2-on cassettes in the absence of LMI070. The quantification of the exon 7 spliced-in or spliced-out transcripts are depicted, and are the mean ± SEM of 6 biological replicates. (FIG. 1D) Representative RT-PCR reaction showing exon 7 inclusion of the indSMN2 cassette in response to different concentrations of LMI070. The quantification of the exon 7 spliced-in or spliced-out transcripts are the relative transcript levels (mean ± SEM) of 9 biological replicates. (FIG. 1E) Firefly luciferase induction of the SMN2 and indSMN2 cassettes in response to LMI070 (100 nM) relative to control DMSO treated cells. The fold change of luciferase activity in LMI070-treated samples relative to DMSO treated control cells is shown. All samples are normalized to Renilla luciferase activity. The data are the mean ± SEM of 8 biological replicates. (FIG. 1F) Exon 7 splicing of the SMN2 cassette in response to LMI070 dose. Representative RT-PCR reaction of exon 7 inclusion as a function of LMI070 dose. The quantification of exon 7 spliced-in or spliced-out transcripts are the relative transcript levels presented as the mean ± SEM of 9 biological replicates. (FIG. 1G) Exon 7 splicing of the indSMN2 cassette in response to RG7800 dose. Representative RT-PCR reaction showing exon 7 inclusion as a function of RG7800 dose. The quantification of the e7 spliced-in or spliced-out transcripts are the relative transcript levels presented as the mean ± SEM of 8 biological replicates. (FIG. 1H) Luciferase activity of the SMN2 and indSMN2 cassettes in response to LMI070. Graph shows relative expression of Firefly luciferase expressed from the SMN2-on or indSMN2-on cassettes in cells treated with DMSO or LMI070 (100 nM). The activity of the transfection control Renilla luciferase cassette is represented as a line above the bar graph. The data are the mean ± SEM of 9 biological replicates.

FIGS. 2A-2N. RNA-Seq for LMI070-responsive pseudo exon discovery. (FIG. 2A) Table showing the top candidate spliced in events identified by RNA-Seq in HEK293 cells treated with 25 nM LMI070. Shown are Gene ID, the LMI070-induced exon and the flanking intron positions, average intron counts, and the U1 LMI070-targeted binding sequence (see SEQ ID NOS: 24-31). (FIG. 2B) Sashimi plot depicting the SF3B3 splicing event in the absence (red) or in response (blue) to 25 nM LMI070. The genomic location of the LMI070 spliced-in exon for SF3B3 is indicated (yellow bar), and the number of intron counts indicated. (FIG. 2C) Sequence logo of the U1 RNA binding sequence targeted by LMI070 from 45 spliced-in exons identified by RNA-Seq. (FIG. 2D) cDNAs amplified from HEK293 cells treated with DMSO or LMI070 shows spliced-in events for SF3B3, BENC1, GXYLT1, C12orƒ4 and PDXDC2, which were confirmed by Sanger sequencing. Asterisks mark nonspecific bands amplified from the PCR reaction. (FIG. 2E) Volcano plot illustrating the differentially expressed genes between DMSO- and LMI070-treated cells. The horizontal bar extending from the y axis represents the significance, 0.05, plotted on a -log10 scale. Thresholds of -0.1 and 0.1 fold-change are indicated by red vertical bars. Genes that meet the threshold for significance and minimum fold change requirements are labeled. (FIGS. 2F-2N) Sashimi plots depicting novel LMI070-spliced in exons for the top ranked genes identified by RNA-Seq. Genomic location and position of the LMI070 spliced in exon are indicated. (FIG. 2F) BENC1, (FIG. 2G) GXYLT1, (FIG. 2H) C12ORF4, (FIG. 2I) PDXDC2, (FIG. 2J) RARS, (FIG. 2K) WNK1, (FIG. 2L) WDR27, (FIG. 2M) CIP2A, and (FIG. 2N) IFT57.

FIGS. 3A-3M. Candidate minigene cassette-responses to LMI070. (FIG. 3A) Cartoon depicting the candidate minigene configuration for controlling translation of Firefly luciferase, with e1 referring in all cases to the exon 5′ of the LMI070-induced pseudoexon from the RNA-Seq analysis (see FIGS. 2B and 2F-2N). A Kozak and ATG initiation codon were positioned within the LMI070-spliced-in exon to initiate translation only in response to drug. (FIG. 3B) Luciferase induction of the minigene cassettes for SF3B3, BENC1, C12ORF4 and PDXDC2. The fold change luciferase activity in LMI070-treated samples (depicted as +) is relative to DMSO-treated (depicted as -) cells, with data normalized to Renilla luciferase. Data are the mean ± SEM of 8 biological replicates. (FIG. 3C) Cartoon depicting the minigene cassette controlling eGFP expression. A Kozak and ATG initiation codon were positioned within the LMI070-induced exon to initiate translation only after treatment with drug. (FIG. 3D) eGFP expression in HEK293 cells transfected with the SF3B3 minigene cassette (X^{on}-eGFP) and treated 24 hr later with DMSO (left) or LMI070 (right). (FIG. 3E) Luciferase activity of the minigene cassettes for SF3B3, BENC1, C12ORF4 and PDXDC2. Data show expression of Firefly luciferase from the minigenes in response to DMSO (depicted as minus) or LMI070 (depicted as plus) treatment relative to Renilla luciferase activity. Data are mean ± SEM of 8 biological replicates. (FIG. 3F) Depiction of the use frequency of the non-AUG start codons (CITE), and those in frame with the luciferase cDNA sequences in transcripts derived from the SF3B3 (see SEQ ID NOS: 16 and 17), BENC1 (see SEQ ID NOS: 18 and 19), C12orƒ4 (see SEQ ID NOS: 20 and 21) or PDXDC2 (see SEQ ID NOS: 22 and 23) minigenes. (FIG. 3G) Representative RT-PCR reaction showing inclusion of the LMI070-induced SF3B3 exon in response to DMSO or LMI070 treatment. Inclusion of the LMI070-spliced in exon was detected using primers binding the exons flanking the LMI070-induced exon (left), or using primers binding within the novel exon sequence (right). (FIG. 3H) Graph depicting the position of predicted enhancer and silencer intronic sequences within the SF3B3 intron. The elements were identified by the Human splicing finder website: (available on the world wide web at umd.be/HSF3/index.html). The position of the LMI070-induced exon (PSEx) and the intronic regions with a high density of silencer sequences are indicated. The grey and red shaded circles indicate intronic regions with a high density of silencer sequences contained with the SF3B3 X^{on} minigenes and the SF3B3int, SF3B3i1, SF3B3i2 and SF3B3i3 cassettes. (FIG. 3I) Cartoon depicting the SF3B3 minigene cassettes containing the original minigene cassette (SF3B3), the full intron sequence (SF3B3int), versions with a high density of intronic silencer sequences (red circles; SF3B3i1, SF3B3i2, SF3B3i3), or a control sequence lacking these same silencers sequences (SF3B3i4). (FIG. 3J) Luciferase activity of various SF3B3 minigene constructs containing additional SF3B3 intron regions. HEK293 cells were transfected with equimolar amounts of plasmids and luciferase activity was determined 24 h after transfection. The graphs show luciferase activity of new SF3B3 cassettes after DMSO (left) or LMI070 (right) treatment and are relative to the original SF3B3 minigene switch (blue and pink for DMSO or LMI070, respectively). Data are the mean ± SEM of 8 biological replicates. (FIG. 3K) Fold-induction of luciferase of the SF3B3 minigene constructs. The fold change of luciferase activity in LMI070-treated samples is relative to DMSO-treated cells. All samples are normalized to Renilla luciferase activity. The original SF3B3 minigene switch is denoted (pink). Data are the mean ± SEM of 8 biological replicates. (FIGS. 3L-3M) Representative gels showing PCR assay for the LMI070-induced pseudoexon. Priming was either to the exons flanking the pseudoexon (FIG. 3L), or within the pseudoexon sequence (FIG. 3M).

FIGS. 4A-4F. Activity of the SF3B3-X^{on} cassette when expressed various promoters and their responsiveness to LMI070 dose. (FIG. 4A) Luciferase induction after transfection of plasmids containing the noted SF3B3-X^{on} -luciferase expression cassettes into HEK293 cells followed by treatment with LMI070 (100 nM, denoted as plus) or treated with DMSO (denoted as minus). All samples are normalized to Renilla luciferase activity and are relative to DMSO treated cells. Data are the mean ± SEM of 8 biological replicates. (FIG. 4B) Luciferase induction in HEK293 cells transfected with plasmids containing the noted SF3B3-X^{on} cassettes and treated with varying doses of LMI070. All samples are normalized to Renilla luciferase activity and are relative to DMSO treated cells (0 nM). Data are the mean ± SEM of 8 biological replicates. (FIG. 4C) Representative gels from RT-PCR analysis for assessment of the LMI070-induced pseudoexons expressed from the noted promoters in response varying doses of LMI070. Pseudoexon inclusion was detected using primers flanking the pseudoexon. Splicing was quantified and transcript levels presented as the mean ± SEM of 8 biological replicates. (FIGS. 4D-4E) Activity of the SF3B3-X^{on} cassette when expressed various promoters and their responsiveness to LMI070 dose. (FIG. 4D) Firefly luciferase from the X^{on} cassettes in response to DMSO or LMI070 treatment (minus, plus, respectively) relative to Renilla luciferase (grey line). (FIG. 4E) Firefly luciferase from the X^{on} cassettes in response to varying doses of LMI070 relative to Renilla luciferase (grey line). The data are the mean ± SEM of 8 biological replicates. (FIG. 4F) Representative gels from RT-PCR analysis for assessment of the LMI070-induced pseudoexons expressed from the noted promoters in response varying doses of LMI070. Pseudoexon inclusion was detected using primers binding within the LMI070-induced pseudoexon and the downstream exon. Splicing was quantified and transcript levels presented as the mean ± SEM of 8 biological replicates.

FIGS. 5A-5X. In vivo activity of X^{on}. (FIG. 5A) Schematic of the in vivo studies using either AAV9.X^{on}.eGFP or AAVPHPeB.X^{on}.eGFP. Mice were injected iv and 4 weeks later were treated with a single dose of 5 or 50 mg/kg LMI070, and tissues harvested 24 h later to assess splicing, transcript levels and protein expression. (FIG. 5B) Representative photomicrograph of liver tissue sections showing eGFP in liver 24 h after treatment with LMI070 at 5 or 50 mg/Kg (scale bar 100 µm). (FIG. 5C) Representative western analysis of eGFP protein levels (2 samples shown; 4 mice/group). β-catenin is shown as loading control. (FIG. 5D) Cartoon depicting the X^{on} assays designed to quantify the LMI070-induced transcripts and eGFP expression levels from the X^{on} cassette after AAV9.X^{on}.eGFP gene transfer. (FIG. 5E) Representative gel from PCR assays demonstrates inclusion of the splicing activity in response LMI070. Data shows the average Ct values for eGFP or LMI070-induced expression using the X^{on} gene expression assays depicted in FIG. 5D. Fold change of the spliced expression cassette is shown relative to basal levels in mice injected with AAV9.X^{on}.eGFP and treated with vehicle. (FIG. 5F) Extended exposure of the western blot from FIG. 5C. (FIG. 5G) Photomicrographs of tissue sections showing eGFP expression in brain from mice treated iv 4 weeks earlier with AAVPHBeB.X^{on}.eGFP, and 24 h after treatment with LMI070 at 5 or 50 mg/kg. eGFP in hippocampus and cortex are shown (scale bar 100 µm). (FIG. 5H) Representative gel from PCR assays demonstrates inclusion of the splicing activity after AAVPHBeB.X^{on}.eGFP gene delivery, in response LMI070. Data shows the average Ct values for eGFP or LMI070-induced expression using the X^{on} gene expression assays depicted in FIG. 5D. Fold change of the spliced expression cassette is shown relative to basal levels in mice injected with AAVPHBeB.X^{on}.eGFP and treated with vehicle. (FIG. 5I) Schematic of the redosing in vivo studies using AAV9.X^{on}.eGFP. Mice were injected iv and 4 weeks later were treated with a single dose of 50 mg/kg LMI070. A group of mice treated with LMI070 were left for one week to washout the drug after which they were redosed. Tissues were harvested 24 h later to assess splicing, transcript levels and protein expression. Predicted eGFP protein levels are also indicated. (FIG. 5J) Representative photomicrograph of liver tissue sections showing eGFP expression in sections harvested from liver 24 h after each dose of LMI070 or vehicle (scale bar 100 µm). (FIG. 5K) Representative western blot of eGFP demonstrating eGFP induction in liver 24 h after dosing with LMI070 or vehicle. β- catenin is shown as loading control. (FIG. 5L) Representative gel from PCR assays demonstrates inclusion of the splicing activity in response LMI070 after each dose. (FIG. 5M) Fold change of the spliced expression cassette in liver tissues over baseline (vehicle treated) is shown in mice injected with AAV9.X^{on}.eGFP and treated with vehicle or drug after each dose. (FIG. 5N) Representative photomicrograph of heart tissue sections showing eGFP expression in heart 24 h after a single dose of LMI070 (50 mg/Kg) (scale bar 200 µm, inset 50 µm). (FIG. 5O) Fold change of the spliced expression cassette is shown relative to basal levels in mice injected with AAV9.X^{on}.eGFP and treated with vehicle in both heart and skeletal muscle tissue. (FIG. 5P) Representative gel from PCR assays demonstrates inclusion of the novel exon after AAV9.X^{on}.eGFP gene delivery in heart and skeletal muscle in response to LMI070. (FIG. 5Q) Extended exposure of the western blot showed on FIG. 5K of eGFP protein levels in liver 24 h after each LMI070 or vehicle dosing. (FIG. 5R) Schematic of the in vivo studies using AAVPHPeB .X^{on}.eGFP to express the X^{on} system in brain.. Mice were injected iv and 4 weeks later were treated with a single dose of 5 or 50 mg/kg LMI070, and brain harvested 24 h later to assess splicing, transcript levels and protein expression. (FIG. 5S) Photomicrographs showing eGFP expression from mice treated iv 4 weeks earlier with AAVPHPeB.X^{on}.eGFP, and 24 h after treatment with vehicle or LMI070 at 5 or 50 mg/kg. eGFP fluorescence is evident in a representative photomicrogrpah of a 40 µm thick sagittal section. (FIG. 5T) Photomicrographs from higher magnifications from sections from of thalamus (Th), hippocampus (Hc), cerebellum (Cb) and facial motor nucleus (VII) from mice injected iv 4 weeks earlier with AAVPHPeB.X^{on}.eGFP, and 24 h after treatment with vehicle or LMI070 at 5 or 50 mg/kg. Scale bar = 100 µm, Insets: scale bar 25 µm) In hippocampus, * and ** there is evidence of expression in the specific hippocampal regions. In cerebellum, * signifies cells that are positive in the deep cerebellar nuclei. (FIG. 5U) Photograph of a representative agarose gel showing PCR amplification demonstrating inclusion of the novel exon after AAVPHPeB.X^{on}.eGFP gene delivery in response to LMI070 taken orally. Data are from tissues harvested from cortex and hippocampus. (FIG. 5V) Data show the average Ct values for eGFP or LMI070-induced expression using the X^{on} cassette. Fold change in cortex and hippocampus of the spliced expression cassette is shown relative to basal levels in mice injected with AAVPHPeB.Xon.eGFP and treated with vehicle. (FIG. 5W) Representative western analysis of eGFP protein levels in cortex and hippocampus samples from mice injected with AAVPHPeB.Xon.eGFP and treated with vehicle or LMI at 5 or 50 mg/Kg., β-catenin is shown as loading control. (FIG. 5X) Photomicrographs showing eGFP expression from mice treated iv 4 weeks earlier with AAVPHPeB.Xon.eGFP, and 24 h after treatment with vehicle or LMI070 at 5 or 50 mg/kg. eGFP in cortex (Cx), Striatum (Str), Substantia Nigra (SN) and medial vestibular nucleus (MV) is shown (Scale bar 100 µm, Insets: scale bar 25 µm).

FIGS. 6A-6M. Regulated SaCas9 editing for Huntington’s disease. (FIG. 6A) Cartoon depicting the allele-specific editing strategy to abrogate mutant HTT expression. SNPs within PAM sequences upstream of HTT exon 1 permit targeted deletions of the mutant allele when present in heterozygosity. After DNA repair, mutant HTT exon 1 is deleted by a pair of sgRNA/Cas9 complexes binding upstream and downstream of exon 1. (FIG. 6B) Cartoon depicting the plasmids used to co-express the sgRNA sequences, SaCas9 (constitutively or with the X^{on} switch), and the selective reporter eGFP/puromycin expression cassettes. (FIG. 6C) Bicistronic plasmids containing the CBh.X^{on}.SaCas9, or the CBhpSaCas9 expression cassette together with CMVeGFP expression cassettes were transfected in HEK293 cells and treated with varying doses of LMI070 and SaCas9 levels determined by western blot 24 h later. eGFP protein levels served as transfection control. Blot is representative of 2 of 4 technical replicates. (FIG. 6D) Schematic of regulated CRISPR for assessing HTT editing. HEK293 cells were transfected with plasmids expressing the sgRNA sequences, SaCas9 (constitutively or via the X^{on} switch), and the selective reporter eGFP/puromycin expression cassettes. Four hours later cells were treated with 100 nM LMI070, and 24 h later with 3 µM Puromycin. After 24 hours of selection cells the Puromycin was removed and cells expanded for 4 days after which cells were collected for genomic DNA, protein and RNA isolation. (FIG. 6E) Representative gel (2 of 5 biological replicates) depicting HTT exon 1-targeted deletion assessed by PCR assay after transfection with constitutive or X^{on}-SaCas9 CRISPR plasmids using control (Ctl) sgRNAs or 935/i3 sgRNA sequences and the selective reporter eGFP/puromycin expression cassettes. (FIG. 6F) Transcript levels as assessed RT-qPCR analysis of HTT mRNA levels from HEK293 cells transfected with constitutive or X^{on}-SaCas9 CRISPR cassettes. Samples are normalized to human GAPDH, and data are the mean ± SEM relative to cells transfected with plasmids containing the constitutive SaCas9 CRISPR cassette expressing a control sgRNA sequence (n = 8 biological replicates; *p < 0.0001, one-way ANOVA followed by a Bonferroni’s post hoc). (FIG. 6G) Representative western blot (2 of 4 biological replicates) for HTT, SaCas9 and eGFP protein levels after puromycin selection and expansion of HEK293 cells transfected with constitutive or X^{on}-SaCas9 CRISPR cassettes. Cells transfected with constitutive cassettes containing sgRNA expression cassette were used as a control. β-catenin served as loading control. (FIG. 6H) Cartoon depicting the allele-specific editing strategy for targeted deletions of mutant HTT exon 1. SNPs within SaCas9 PAM sequences upstream of HTT exon-1 allow selective editing of the mutant allele when present in heterozygosity. After DNA repair, mutant HTT exon 1 is removed by a pair of sgRNA/SaCas9 complexes binding upstream and downstream of exon 1. The SNP (referred to as 935) is annotated. (FIG. 6I) HTT mRNA levels in HEK293 cells transfected with constitutive SaCas9 CRISPR cassettes targeting HTT exon 1 flanking sequences as assessed by RT-qPCR. Samples are normalized to human GAPDH, and data are the mean ± SEM relative to cells transfected with SaCas9 CRISPR expression plasmids together with the control sgRNA sequence (n = 8 biological replicates; *p < 0.0001, one-way ANOVA followed by a Bonferroni’s post hoc). (FIG. 6J) Representative gel showing HTT exon-1-targeted deletion in cells transfected with SaCas9 CRISPR expression plasmids together with the control sgRNA sequence or sgRNA sequences targeting the flanking sequences of HTT exon 1 and the selective reporter eGFP/puromycin expression cassettes. The Sanger sequencing results of the small PCR products confirmed deletion of HTT exon 1 at the DNA cleavage site. (FIG. 6K) A representative western blot showing HTT exon 1 levels in cells transfected with SaCas9 CRISPR plasmids and the noted sgRNAs. SaCas9 and b catenin was used as protein loading controls, respectively. (FIG. 6L) Extended exposure of blot from FIG. 6B. (FIG. 6M) Extended exposure of SaCas9 expression from FIG. 6G.

FIGS. 7A-7H. (FIG. 7A) Schematic of the in vivo studies using AAV8.X^{on}.mEpo. Mice were injected iv and 4 weeks later were treated with three consecutive doses of LMI070. Mouse Epo and hematocrit percentages were determined at different time points before and after treatment with LMI070 or vehicle, as indicated (FIG. 7B) Mouse Epo (pg/ml) (left) and hematocrit levels (right) were determined before (basal) or after LMI070 or vehicle treatment. (FIG. 7C) Schematic of the in vivo studies using AAV8.X^{on}.SaCas9 and AAV8sgAi9.eGFP vectors. Ai14 mice were injected iv and 4 weeks later were treated with a single dose of LMI070 (25 mg/ml). Mice were euthanized 1 week after SaCas9 induction with LMI070 to determine the extent of editing of the Ai14 reporter sequence in the mouse genome. (FIG. 7D) Demonstration of genomic DNA editing via PCR of liver lysates from Ai14 reporter mice injected with AAV8.X^{on}.SaCas9 + AAV8.sgAi9 eGFP vectors. Editing is evident in response to LMI070 (FIG. 7E) Photomicrographs showing eGFP and tdTomato expression from mice treated iv with AAV8.Xon.SaCas9 + AAV8.sgAi9 eGFP vectors, after treatment with vehicle or LMI070 at 25 mg/kg. (FIG. 7F) Schematic of the in vivo redosing studies using AAV8.X^{on}.mEpo. LMI070 redosing was redosed after hematocrite levels returned to basal levels. (FIG. 7G) Epo levels (pg/ml) in response to LMI070 after the first treatment (FIG. 7H) Time course of the mouse hematocrit levels after treatment with LMI070 or vehicle.

FIGS. 8A-8F. (FIG. 8A) Cartoon depicting the structure and size of the SF3B3.Xon and the SF3B3.Xon100 cassette. Size of the novel exon flanking introns sequences are indicated. (FIG. 8B) Firefly luciferase induction of the SF3B3.Xon and the SF3B3.Xon100 cassette in response to LMI070 relative to Renilla luciferase. The data are the mean ± SEM of 8 biological replicates. (FIG. 8C) Luciferase activity of the SF3B3.Xon.100 and SF3B3.Xon cassettes. Data show expression of Firefly luciferase from the minigenes in response to DMSO or LMI070 treatment relative to Renilla luciferase activity. Data are mean ± SEM of 8 biological replicates. (FIG. 8D) Luciferase expression from the SF3B3.Xon.100 cassette relative to expression of the SF3B3.X^{on}. Data show expression of SF3B3.X^{on}100 is comparable to the expression of SF3B3.X^{on} in the off and the on state. (FIG. 8E) Representative RT-PCR data showing LMI070-induced SF3B3 exon inclusion in response to DMSO or LMI070 treatment in the SF3B3.X^{on}100 and the SF3B3.X^{on}. The LMI070-spliced-in exon was detected using primers flanking the LMI070-induced exon. (FIG. 8F) Representative RT-PCR data showing LMI070-induced SF3B3 exon inclusion in response to DMSO or LMI070 treatment in the SF3B3.X^{on}100 and the SF3B3.X^{on}. Inclusion of the LMI070-spliced in exon was detected using one primer within the novel exon sequence and one in a flanking exon.

FIGS. 9A-9D. (FIG. 9A) Schematic of the in vivo studies using AAVPHPeB.X^{on}.SaCas9 and AAVPHPeB sgAi9.eGFP vectors. Ai14 mice were injected iv and 4 weeks later were treated with three doses of LMI070 (25 mg/ml). Mice were euthanized 1 week after SaCas9 induction with LMI070 to determine editing of the Ai14 reporter sequence in the mouse genome. (FIG. 9B) Photomicrographs showing tdTomato expression in cortex and hippocampus from mice treated iv with AAVPHPeB.Xon.SaCas9 + AAVPHPeB.sgAi9 eGFP vectors, after treatment with vehicle or three doses of LMI070 at 25 mg/kg. (FIG. 9C) Schematic of the in vivo studies using AAVPHPeB.X^{on}.SaCas9 and AAVPHPeB sg4/i3.eGFP vectors. BacHD mice were injected iv and 3 weeks later were treated with three doses of LMI070 (25 mg/ml). Mice were euthanized 3 week after SaCas9 induction with LMI070 to determine editing of the Huntingtin locus. (FIG. 9D) Representative RT-PCR data showing LMI070-induced SF3B3 exon inclusion in response to LMI070 treatment in BacHD mice 24 h after the last treatment, relative to vehicle treated animals.

FIGS. 10A-10B. Optimized minigene cassette for secreted proteins -responses to LMI070 (FIG. 10A) eGFP expression in HEK293 cells transfected with the optimized SF3B3 minigene cassettes and treated 24 hr later with DMSO (left) or LMI070 (right). (FIG. 10B) Representative RT-PCR reaction showing inclusion of the LMI070-induced SF3B3 exon in response to DMSO or LMI070 treatment in the optimized minigene. Inclusion of the LMI070-spliced in exon was detected using primers binding the exons flanking the LMI070-induced exon (left), or one primer binding within the novel exon sequence and one binding a flanking exon (right).

FIGS. 11A-11O. Minigene sequences. Upstream exon (Exon 1 or e1), novel exon (Exon2 or e2) and downstream exon (Exon 3 or e3) are shown in bolded letters. The sequence between upstream exon and novel exon is Intron 1, and the sequence between novel exon and downstream exon is Intron 2. Start codons are underlined with solid lines. Kozak sequences are underlined with dash lines. (FIG. 11A) The SMN2 minigene sequence. (FIG. 11B) The SMN2ind minigene sequence. (FIG. 11C) The SMN2act minigene sequence. (FIG. 11D) The SF3B3 minigene sequence. (FIG. 11E) The SF3B3int minigene sequence. (FIG. 11F) The SF3B3int1 minigene sequence. (FIG. 11G) The SF3B3int2 minigene sequence. (FIG. 11H) The SF3B3i3 minigene sequence. (FIG. 11I) The SF3B3i4 minigene sequence. (FIG. 11J) The Benc1 minigene sequence. (FIG. 11K) The C12orf4 minigene sequence. (FIG. 11L) The PDXDC 2 minigene sequence. (FIG. 11M) The SF3B3.OPTX^{ON}NGS minigene sequence. (FIG. 11N) The SF3B3.OPTX^{ON}KGS minigene sequence. (FIG. 11O) The SF3B3.OPTX^{ON}RGS minigene sequence. In FIGS. 11M-O, the initial coding sequence encoding the N-terminal portion of the signal peptide is boxed and the codon encoding for the N, K, or R, respectively, is double underlined.

### DETAILED DESCRIPTION

To date, gene therapies for human application rely on engineered promoters that cannot be finely controlled. Provided herein are universal switch elements that allows precise control for gene silencing or gene replacement after exposure to a small molecule. Importantly, these small molecule inducers are in human use, are orally bioavailable when given to animals or humans, and can reach both peripheral tissues and the brain. Moreover, the switch system (X^{on}) does not require the co-expression of any regulatory proteins. Using X^{on}, translation of desired elements for gene knockdown or gene replacement occurs after a single oral dose, and expression levels can be controlled by drug dose or in waves with repeat drug intake. This universal switch can provide temporal control of gene editing machinery and gene addition cassettes that can be adapted to cell biology applications and animal studies. Additionally, due to the oral bioavailability and safety of the drugs employed, the X^{on} switch provides an unprecedented opportunity to refine gene therapies for more appropriate human application.

### I. Alternative Splicing-regulated Transgene Expression

Disclosed herein are chimeric minigenes, where the alternative splicing of the minigene determines whether the downstream encoded gene is expressed. The encoded gene may be an inhibitory RNA, a CRISPR-Cas9 protein, a therapeutic protein, or a transactivator.

In one example, the minigene comprises three exons, Exons 1-3, and Exon 2 is skipped in the basal state. When Exon 2 is skipped, the downstream encoded gene is not produced because the translation initiation regulatory sequences are located in Exon 2. As such, translation of the encoded protein is not initiated. In order to turn on expression of the encoded gene, the inclusion of the skipped exon must be induced. Such can occur as a result of the presence of a small molecule splicing modifier. For example, the minigene may comprise Exons 6-8 of the SMN2 gene, in which case Exon 7 is skipped in the basal state. However, Exon 7 is included in the presence of certain splicing modifier small molecules (e.g., LMI070 or RG7800/RG7619). As such, the downstream encoded gene will be expressed in the presence of LMI070 or RG7800/RG7619, but not in its absence. As another example, the minigene may comprise an upstream exon and a downstream exon from SF3B3, in addition to an intervening pseudoexon, in which case the pseudoexon is skipped in the basal state. However, the pseuodexon is included in the presence of certain splicing modifier small molecules (e.g., LMI070 or RG7800/RG7619). As such, in both of these examples, the downstream encoded gene will be expressed in the presence of LMI070 or RG7800/RG7619, but not in its absence.

As another alternative, inclusion of the skipped exon may be induced by a certain disease state. For example, Huntington’s disease results in the generation of transcript isoforms generated by alternative splicing. As such, the minigene may comprise exons from a gene whose splicing is altered in Huntington’s disease, i.e., when mutant HTT is expressed, such that an exon that is normally skipped in a healthy cell is included instead (e.g., PCDH1 (5 : 141869432 - 141878222). If needed, a stop codon may be engineered into the exon downstream of the alternatively spliced exon to ensure that no encoded gene is expressed in non-diseased cells. The result is that the encoded gene will only be expressed when mutant HTT is present. In this example, the encoded gene may encode an inhibitory RNA that knocks down the expression of mutant HTT, thus creating an autoregulatory feedback loop-the presence of mutant HTT will induce expression of an inhibitory RNA that targets mutant HTT, thereby reducing mutant HTT levels to a level that causes the splicing of the minigene to return to the non-diseased state, thereby turning off the expression of the inhibitory RNA and allowing for expression of mutant HTT, which will reach a level that induces expression of the inhibitory RNA, and so on. Alternatively, the target gene may encode a CRISPR-Cas9 system that represses the transcription of the HTT gene.

The expression of the chimeric minigene may be regulated by various types of promoters, depending on the desired expression pattern. For example, the promoter may be a universally constitutive promoter, such as a promoter for a housekeeping gene (e.g., ACTB). As another example, the promoter may be a cell-type specific promoter, such as the promoter for synapsin for neuronal expression. As yet another example, the promoter may be an inducible promoter.

The chimeric minigene may have a cleavable peptide located between the minigene and the encoded gene. In some cases, the cleavable peptide may be a self-cleavable peptide, such as, for example, a 2A peptide. The 2A peptide may be a T2A peptide, a P2A peptide, an E2A peptide, or a F2A peptide. The presence of this peptide provides for separation of the minigene-encoded peptide from the encoded protein following translation. In some cases, the cleavable peptide may be a cleavage site for a widely expressed, endogenous endoprotease, such as, for example, furin, prohormone convertase 7 (PC7), paired basic amino-acid cleaving enzyme 4 (PACE4), or subtilisin kexin isozyme 2 (SKI-1). In some cases, the cleavable peptide may be a cleavage site for a tissue-specific or cell-specific endoprotease (such as, e.g., prohormone convertase 2 (PC2; primarily expressed in endocrine tissue and brain), prohormone convertase ⅓ (PC⅓; primarily expressed in endocrine tissue and brain), prohormone convertase 4 (PC4; primarily expressed in the testis and ovary), and proprotein convertase subtilisin kexin 9 (PSCK9; primarily expressed in the lung and liver)).

### II. LMI070-induced Pseudoexons

Tables 1A-1E provide the genomic locations of candidate LMI070-induced exons and the frequency of events from RNA-Seq datasets. Summarized here are the differentially expressed candidate LMI070-induced splicing positions as identified by RNA-Seq of HEK293 cells treated with either DMSO or LMI070 (25 nM). All candidates shown were manually selected from a bioinformatically generated list of top hits based on their suitability for construction of an exon switching genomic minigene, their exclusivity to the LMI070 condition, and their minimal to undetectable levels in DMSO treated cells. The top 25 rows of each table indicate hits observed exclusively upon LMI070 exposure. The following 22 rows of each table indicate candidates where splicing was enriched but not totally exclusive to LMI070 treatment.

Table 1A provides the GRCh38 genomic positions used to create the splice event-containing genomic minigene, the GRCh38 genomic positions of the pseudoexon created by LMI070-induced splicing, the number of exon-exon junction spanning reads observed with DMSO treatment, and the number of exon-exon junctions spanning reads observed with LMI070 treatment. To assess the frequency with which LMI070-induced events occur we queried Introlopolis (Nellore et al., 2016), a database containing the frequency of splicing events observed across 21,504 human RNA-Seq samples, representing a diverse set of human tissues and conditions. The reference genome used for the Intropolis database is GRCh37 so the LiftOver feature from the UCSC genome browser was used to convert the GRCh38 coordinates to GRCh37.

Table 1B provides the GRCh37 genomic position of each minigene, the GRCh37 genomic position of the pseudoexon, and the DNA sequence of the LMI070 binding sequence in the pseudoexon.

Table 1C provides the position of canonical splice junction (CJ), the number of Intropolis RNA-Seq datasets in which each canonical splice event was observed, and the total number of observations identified for each canonical splice site.

Table 1D provides the position of junction 1 (J1) and the first LMI070-induced exon-exon junction (sorted by genomic position) connecting a canonical exon to a LMI070-induced pseudoexon. 14 and 15 indicate the number and percentage of Intropolis datasets in which each S1 splice event was observed. Also indicated are the number and percentage of total counts in which each S1 splice event was observed.

Table 1E provides the position of junction 2 (J2) the second LMI070 induced exon-exon junction (sorted by genomic position) connecting a LMI070 induced pseudoexon to a canonical exon. Also listed are the number and percentage of Intropolis datasets in which the LMI070 induced splicing event was observed. Also indicates are the total number and percentage of reads containing each junction in the Intropolis dataset.

TABLE 1A

GRCh38 genomic minigenes

| | Gene ID | Genomic Minigene region (GRCh38) | Pseudo exon position (GRCh38) | DMSO Observed Avg Counts (J1, J2) | LMI070 Observed Avg Counts (J1, J2) |
|---|---|---|---|---|---|
| Exclusive | SF3B3 | chr16:70,526,657-70,529,199 | chr16:70,527,376-70,527,429 | 0, 0 | 31, 30.5 |
| | BENC1 | chr17:42,810,759-42,811,797 | chr17:42,811,292-42,811,330 | 0, 0 | 24.45, 15.75 |
| | GXYLT1 | chr12:42,087,786-42,097,614 | chr12:42,095,151-42,095,214 | 0, 0 | 10.75, 23.75 |
| | SKP1 | chr5:134,173,809-134,177,053 | chr5:134,175,284-134,175,385 | 0, 0 | 5.75, 23.75 |
| | SKP1 | chr5:134,173,809-134,177,053 | chr5:134,175,284-134,175,423 | 0, 0 | 15.25, 11.75 |
| | C12orƒ4 | chr12:4,536,017-4,538,508 | chr12:4,537,380-4,537,514 | 0, 0 | 17.5, 19 |
| | SSBP1 | chr7:141,739,167-141,742,229 | chr7: 141,741,310-141,741,459 | 0, 0 | 17.25, 2.25 |
| | RARS | chr5:168,517,815-168,519,190 | chr5:168,518,369-168,518,523 | 0, 0 | 13.5, 1.5 |
| | RARS | chr5:168,517,815-168,519,190 | chr5:168,518,469-168,518,523 | 0, 0 | 13.6, 1.75 |
| | PDXDC2P | chr16:70,030,988-70,031,968 | chr16:70,031,186-70,031,248 | 0, 0 | 13.25, 10.25 |
| | STRADB | chr2:201,469,953-201,473,076 | chr2:201,470,907-201,471,111 | 0, 0 | 9.5, 5.25 |
| | WNK1 | chr12:894,562-896,732 | chr12:895,161-895,196 | 0, 0 | 9, 5.5 |
| | WDR27 | chr6:169,660,663-169,662,424 | chr6:169,661,703-169,661,750 | 0, 0 | 8.5, 7.25 |
| | CIP2A | chr3:108,565,355-108,566,638 | chr3:108,565,898-108,565,931 | 0, 0 | 7.75, 5 |
| | ITF57 | chr3:108,191,521-108,206,696 | chr3:108,192,476-108,192,526 | 0, 0 | 7.25, 5.25 |
| | HTT | chr4:3,212,555-3,214,145 | chr4:3213622-3213736 | 0, 0 | 7, 2.25 |
| | SKA2 | chr17:59,112,228-59,119,514 | chr17:59119395-59119495 | 0, 0 | 6.75, 1 |
| | EVC | chr4:5,733,318-5,741,822 | chr4:5741334-5741441 | 0, 0 | 6.5, 3 |
| | DYRK1A | chr21:37,420,144-37,473,056 | chr21:37422581-37422652 | 0, 0 | 6.25,6 |
| | GNAQ | chr9:77,814,652-77,923,557 | chr9:77920648-77920703 | 0, 0 | 6, 1 |
| | ZMYM6 | chr1:35,019,257-35,020,472 | chr1:35020261-35020279 | 0, 0 | 5.75, 4 |
| | CYB5B | chr16:69,448,031-69,459,260 | chr16:69448605-69448753 | 0, 0 | 5.75, 1.25 |
| | MMS22L | chr6:97,186,342-97,229,533 | chr6:97201362-97201465 | 0, 0 | 5.75, 2 |
| | MEMO1 | chr2:31,883,262-31,892,301 | chr2:31,887,035-31887087 | 0, 0 | 5, 2.25 |
| | PNISR | chr6:99,416,278-99,425,413 | chr6:99420523-99420584 | 0, 0 | 5, 4 |
| Exclusive | CACNA2D1 | chr7:82,066,406-82,084,958 | chr7:82,076,016-82,076,122 | 0.25, 0.75 | 18.5, 1.5 |
| | SSBP1 | chr7:141,739,083-141,742,248 | chr7:141741310-141741459 | 0.25,0 | 16.75, 2.25 |
| | DDX42 | chr17:63,805,048-63,806,672 | chr17:63,806,151-63,805,994 | 0.25,0 | 13.75, 4.5 |
| | ASAP1 | chr8:130,159,817-130,167,688 | chr8:130,160,785-130,160,793 | 0.25, 0.25 | 13, 11.5 |
| | DUXAP10 | chr14:19,294,564-19,307,199 | chr14:19,305,354-19,305,469 | 0.25, 0.75 | 9.5, 1.25 |
| | AVL9 | chr7:32,558,783-32,570,372 | chr7:32,562,558-32,562,913 | 0.25, 0.5 | 7.5, 1.5 |
| | DYRKIA | chr21:37,419,920-37,472,960 | chr21:37,422,582-37,422,652 | 0.25,0 | 6.25,6 |
| | FAM3A | chrX:154,512,311-154,512,939 | chrX: 154,512,568-154,512,706 | 0.25,0 | 5.75, 1 |
| | FHOD3 | chr18:36,740,620-36,742,886 | chr18:36,742,377-36,742,468 | 0.5, 0.25 | 15.25, 3.5 |
| | TBCA* | chr5:77,707,994-77,777,000 | chr5:77,774,217 | 0.5 | 14 |
| | MZT1 | chr13:72,718,939-72,727,611 | chr13:72,725,642-72,725,778 | 0.5, 1 | 13.25, 1.25 |
| | LINC01296 | chr14:19,092,877-19,096,652 | chr14:19094556-19094671 | 0.5, | 8.25, |
| | SF3B3 | chr16:70,541,627-70,544,553 | chr16:70544169-70544249 | 0.5, 0 | 8.25, 3 |
| | SAFB | chr19:5,654,060-5,654,457 | chr19:5,654,140-5,654,368 | 0.5, 0 | 6.25,2 |
| | GCFC2 | chr2:75,702,163-75,706,652 | chr2:75,702,691-75,702,807 | 0.5, 0 | 6.25,2 |
| | MRPL45 | chr17:38,306,450-38,319,088 | chr17:38,312,587-38,312,661 | 0.5, 0 | 5.75, 1.25 |
| | SPIDR | chr8:47,260,788-47,280,196 | chr8:47,273,337-47,273,450 | 0.5, 0 | 5.5, 1.75 |
| | DUXAP8 | chr22:15,815,315-15,828,713 | chr22:15,817,119-15,817,234 | 0.75, 0.25 | 13.75, 1.25 |
| | PDXDC1 | chr16:15,008,772-15,009,763 | chr16:15,009,499-15,009,561 | 2, 1 | 16.75, 1.5 |
| | MAN1A2 | chr1:117,442,104-117,461,030 | chr1:117,456,085-117,456,206 | 0.75, 1 | 8, 5.25 |
| | RAF1 | chr3:12,600,376-12,604,350 | chr3:12,603,478-12,603,537 | 1, 0.25 | 16.5, 1 |
| | ERGIC3 | chr20:35,548,787-35,554,452 | chr20:35,549,163-35,549,207 | 1, 0 | 7.5, 2.5 |

TABLE 1B

GRCh37 (hg19) genomic minigenes

| | | | | |
|---|---|---|---|---|
| | Gene ID | Genomic Minigene region (GRCh37; hg19) | Pseudo exon position (GRCh37; hg19) | LMI070 Binding sequence |
| Exclusive | SF3B3 | chr16:70560560-70563102 | chr16:70561279-70561332 | AGAGTAAGAC |
| | BENC1 | chr17:40962777-40963815 | chr17:40963310-40963348 | AGAGTAAGGC |
| | GXYLT1 | chr12:42481588-42491416 | chr12:42488953-42489016 | AGAGTATAGT |
| | SKP1 | chr5:133509500-133512744 | chr5:133510975-133511076 | AGAGTAGGAT |
| | SKP1 | chr5:133509500-133512744 | chr5:133510975-133511114 | AGAGTAGGAT |
| | C12orƒ4 | chr12:4645183-4647674 | chr12:4646546-4646680 | AGAGTAAGAA |
| | SSBP1 | chr7:141438967-141442029 | chr7:141441110-141441259 | AGAGTAAGGC |
| | RARS | chr5:167944820-167946195 | chr5:167945374-167945528 | AGAGTAGGAT |
| | RARS | chr5:167944820-167946195 | chr5:167945474-167945528 | AGAGTAGGAT |
| | PDXDC2P | chr16:70064891-70065871 | chr16:70065089-70065151 | AGAGTAAGAA |
| | STRADB | chr2:202334676-202337799 | chr2:202335630-202335834 | AGAGTAAGGA |
| | WNK1 | chr12:1003728-1005898 | chr12:1004327-1004362 | AGAGTAGGTG |
| | WDR27 | chr6:170060759-170062520 | chr6:170061799-170061846 | AGAGTAAGCA |
| | CIP2A | chr3:108284202-108285485 | chr3:108284745-108284778 | AGAGTAAGAA |
| | ITF57 | chr3:107910368-107925543 | chr3:107911323-107911373 | AGAGTAGGCC |
| | HTT | chr4:3214282-3215872 | chr4:3215349-3215463 | AGAGTAAGGG |
| | SKA2 | chr17:57189589-57196875 | chr17:57196756-57196856 | AGAGTAAGAG |
| | EVC | chr4:5735045-5743549 | chr4:5743061-5743168 | AGAGTAAGCA |
| | DYRKIA | chr21:38792446-38845358 | chr21:38794883-38794954 | AGAGTAGGTT |
| | GNAQ | chr9:80429568-80538473 | chr9:80535564-80535619 | AGAGTAAGCT |
| | ZMYM6 | chr1:35484858-35486073 | chr1:35485862-35485880 | ACTGTGAGTA |
| | CYB5B | chr16:69481934-69493163 | chr16:69482508-69482656 | TAGGTGGTTC |
| | MMS22L | chr6:97634218-97677409 | chr6:97649238-97649341 | GAGGTGATTG |
| | MEMO1 | chr2:32108331-32117370 | chr2:32112104-32112156 | AGAGTAAGGT |
| | PNISR | chr6:99864154-99873289 | chr6:99868399-99868460 | AGAGTAGTGT |
| Exclusive | CACNA2D1 | chr7:81695722-81714274 | chr7:81705332-81705438 | CAGGTTGGTA |
| | SSBPI | chr7:141438883-141442048 | chr7:141441110-141441259 | AGAGTAAGGC |
| | DDX42 | chr17:61882408-61884032 | Unable to lift over | AGAGTAAGAT |
| | ASAP1 | chr8:131172063-131179934 | chr8:131173031-131173039 | AGAGTAAGTA |
| | DUXAP10 | chr14:19882243-19894878 | chr14:19893035-19893150 | AGAGTAAGGT |
| | AVL9 | chr7:32598395-32609984 | chr7:32602170-32602525 | AGAGTAAGAC |
| | DYRKIA | chr21:38792222-38845262 | chr21:38794884-38794954 | AGAGTAGGTT |
| | FAM3A | chrX:153740635-153741263 | chrX:153740892-153741030 | GGGGTAGGGA |
| | FHOD3 | chr18:34320583-34322849 | chr18:34322340-34322431 | AGAGTAAGAG |
| | TBCA ^{∗} | chr5:77003819-77072824 | chr5:77070041 | ND |
| | MZT1 | chr13:73293077-73301749 | chr13:73299780-73299916 | AGAGTAAGAA |
| | LINC01296 | chr14:19680556-19684333 | chr14:19682237-19682352 | AGAGTAAGAT |
| | SF3B3 | chr16:70575530-70578456 | chr16:70578072-70578152 | AGAGTAAAGA |
| | SAFB | chr19:5654071-5654468 | chr19:5654151-5654379 | AGAGTAAGGA |
| | GCFC2 | chr2:75929289-75933778 | chr2:75929817-75929933 | TGAGTAAGAG |
| | MRPL45 | chr17:36462417-36474972 | chr17:36468550-36468624 | AGAGTAAGAC |
| | SPIDR | chr8:48173380-48192784 | chr8:48185929-48186042 | AGAGTAAGAC |
| | DUXAP8 | chr14:19,680,685-19,691,354 | chr14:19682237-19682352 | AGAGTAAGAT |
| | PDXDC1 | chr16:15102629-15103620 | chr16:15103356-15103418 | AGAGTAAGAA |
| | MANIA2 | chr1:117984726-118003652 | chr1:117998707-117998828 | AGAGTAAGGT |
| | RAF1 | chr3:12641875-12645849 | chr3:12644977-12645036 | AGAGTAGGTA |
| | ERGIC3 | chr20:34136540-34142223 | chr20:34136917-34136961 | GTGGTAGGTA |

TABLE 1C

GRCh37 (hg19) - Canonical Junction Metrics

| | Gene ID | CJ exon-exon junction | CJ Intropolis Datasets | CJ_TotalCounts |
|---|---|---|---|---|
| Exclusive | SF3B3 | chr16:70560630-70562775 | 12873 | 825337 |
| | BENC1 | chr17:40962947-40963672 | 13635 | 910210 |
| | GXYLT1 | chr12:42481750-42491243 | 7430 | 43383 |
| | SKP1 | chr5:133509714-133512545 | 13277 | 2756168 |
| | SKP1 | chr5:133509714-133512545 | 13277 | 2756168 |
| | C12orf4 | chr12:4645386-4647574 | 9856 | 102163 |
| | SSBP | chr7:141438991-141441968 | 14501 | 2473164 |
| | RARS | chr5:167945068-167946085 | 13602 | 826669 |
| | RARS | chr5:167945068-167946085 | 13602 | 826669 |
| | PDXDC2P | chr16:70064970-70065802 | 9586 | 117767 |
| | STRADB | chr2:202334776-202337677 | 9420 | 155599 |
| | WNK1 | chr12:1003802-1005236 | 12935 | 800572 |
| | WDR27 | chr6:170060863-170062399 | 9937 | 128203 |
| | CIP24 | chr3:108284302-108285343 | 9210 | 124822 |
| | 1TF57 | chr3:107910491-107925474 | 12662 | 583056 |
| | HTT | chr4:3214437-3215684 | 11146 | 243427 |
| | SKA2 | chr17:57189707-57196679 | 10377 | 297301 |
| | EVC | chr4:5735163-5743442 | 6716 | 74348 |
| | DYRK1A | chr21:38792687-38844985 | 10821 | 152058 |
| | GNAQ | chr9:80430687-80537076 | 10041 | 224120 |
| | ZMYM6 | chr1:35485204-35485983 | 11692 | 199379 |
| | CYB5B | chr16:69482048-69492995 | 13834 | 1150919 |
| | MMS22L | chr6:97634567-97676769 | 8959 | 91164 |
| | MEMO1 | chr2:32108532-32117060 | 8678 | 99228 |
| | PNISR | chr6:99864305-99873090 | 13184 | 888372 |
| Exclusive | CACNA2D | chr7:81695841-81714084 | 6256 | 96528 |
| | SSBP1 | chr7:141438991-141441968 | 14501 | 2473164 |
| | DDX42 | chr17:61882536-61883894 | 12718 | 722896 |
| | ASAP1 | chr8:131172211-131179781 | 11448 | 396871 |
| | DUXAP1O | chr14:19884030-19894699 | 638 | 1502 |
| | AVL9 | chr7:32599077-32609631 | 11339 | 199392 |
| | DYRK1A | chr21:38792687-38844985 | 10821 | 152058 |
| | FAM3A | chrX:153740736-153741146 | 9459 | 98652 |
| | FHOD3 | chr18:34320802-34322699 | 7279 | 113308 |
| | TBCA ^{∗} | chr5:77004173-77072028 | 14180 | 942768 |
| | MZT | chr13:73293236-73301661 | 12409 | 516948 |
| | LINC01296 | chr14:19680686-19683691 | 239 | 315 |
| | SF3B3 | chr16:70575738-70578340 | 12928 | 880623 |
| | SAFB | chr19:5654212-5654378 | 13168 | 1083231 |
| | GCFC2 | chr2:75929550-75933648 | 11225 | 168121 |
| | MRPL45 | chr17:36462599-36474585 | 12718 | 608565 |
| | SPIDR | chr8:48173584-48192449 | 10729 | 158491 |
| | DUXAP8 | chr14:19680686-19683691 | 239 | 315 |
| | PDXDC1 | chr16:15102705-15103537 | 12939 | 767189 |
| | MAN1A2 | chr1:117984948-118003110 | 11582 | 229559 |
| | RAF1 | chr3:12641915-12645634 | 12808 | 832362 |
| | ERGIC3 | chr20:34136619-34142142 | 4423 | 84287 |

TABLE 1D

GRCh37 (hg19) - LMI070 Junction1 Metrics

| | Gene ID | J1 Position | J1 NumDatasets | J1 % Data Sets | J1 TotalCounts | J1 % Total counts |
|---|---|---|---|---|---|---|
| Exclusive | SF3B3 | chr16:70560630-70561278 | 10 | 0.08 | 13 | 0.002 |
| | BENC1 | chr17:40962947-40963309 | 301 | 2.21 | 552 | 0.061 |
| | GXYLT1 | chr12:42481750-42488952 | 8 | 0.11 | 9 | 0.021 |
| | SKP1 | chr5:133509714-133510974 | 15 | 0.11 | 16 | 0.001 |
| | SKP1 | chr5:133509714-133510974 | 15 | 0.11 | 16 | 0.001 |
| | C12orf4 | chr12:4645386-4646545 | 354 | 3.59 | 563 | 0.551 |
| | SSBP1 | chr7:141438991-141441109 | 38 | 0.26 | 71 | 0.003 |
| | RARS | chr5:167945068-167945373 | 20 | 0.15 | 21 | 0.003 |
| | RARS | chr5:167945068-167945473 | 2 | 0.01 | 2 | 0.000 |
| | PDXDC2P | chr16:70064970-70065088 | 284 | 2.96 | 587 | 0.498 |
| | STRADB | chr2:202334776-202335629 | 150 | 1.59 | 189 | 0.121 |
| | WNK1 | chr12:1003802-1004326 | 30 | 0.23 | 39 | 0.005 |
| | WDR27 | chr6:170060863-170061798 | 1322 | 13.30 | 2407 | 1.877 |
| | CIP2A | chr3:108284302-108284744 | 126 | 1.37 | 181 | 0.145 |
| | ITF57 | chr3:107910491-107911322 | 64 | 0.51 | 150 | 0.026 |
| | HTT | chr4:3214437-3215348 | 452 | 4.06 | 599 | 0.246 |
| | SKA2 | chr17:57189707-57196756 | 373 | 3.59 | 605 | 0.203 |
| | EVC | chr4:5735163-5743060 | 86 | 1.28 | 120 | 0.161 |
| | DYRK1A | chr21:38792687-38794883 | 29 | 0.27 | 51 | 0.034 |
| | GNAQ | chr9:80430687-80535563 | 23 | 0.23 | 28 | 0.012 |
| | ZMYM6 | chr1:35485204-35485861 | 312 | 2.67 | 1041 | 0.522 |
| | CYB5B | chr16:69482048-69482509 | 191 | 1.38 | 286 | 0.025 |
| | MMS22L | chr6:97634567-97649237 | 41 | 0.46 | 58 | 0.064 |
| | MEMO1 | chr2:32108532-32112103 | 167 | 1.92 | 230 | 0.232 |
| | PNISR | chr6:99864305-99868398 | 1 | 0.01 | 1 | 0.000 |
| Enriched | CACNA2D1 | chr7:81695 841-81705331 | 1 | 0.02 | 1 | 0.001 |
| | SSBP1 | chr7:141438991-141441109 | 38 | 0.26 | 71 | 0.003 |
| | DDX42 | NA | NA | NA | NA | NA |
| | ASAP1 | chr8:131172211-131173030 | 2429 | 21.22 | 16818 | 4.238 |
| | DUXAP10 | Not Found | Not Found | 0.00 | Not Found | 0.000 |
| | AVL9 | chr7:32599077-32602169 | 2728 | 24.06 | 6204 | 3.111 |
| | DYRK1A | chr21:38792687-38794883 | 29 | 0.27 | 51 | 0.034 |
| | FAM3A | chrX:153740736-153740891 | 351 | 3.71 | 481 | 0.488 |
| | FHOD3 | chr18:34320802-34322339 | 210 | 2.89 | 295 | 0.260 |
| | TBCA* | NA | NA | NA | NA | NA |
| | MZT1 | chr13:73293236-73299779 | 51 | 0.41 | 103 | 0.020 |
| | LINC01296 | chr14:19680686-19682236 | 20 | 8.37 | 22 | 6.984 |
| | SF3B3 | chr16:70575738-70578071 | 233 | 1.80 | 467 | 0.053 |
| | SAFB | Not Found | Not Found | 0.00 | Not Found | 0.000 |
| | GCFC2 | chr2:75929550-75929816 | 124 | 1.10 | 195 | 0.116 |
| | MRPL45 | chr17:36462599-36468549 | 778 | 6.12 | 1191 | 0.196 |
| | SPIDR | chr8:48173584-48185928 | 414 | 3.86 | 568 | 0.358 |
| | DUXAP8 | chr14:19680686-19682236 | 20 | 8.37 | 22 | 6.984 |
| | PDXDC1 | chr16:15102705-15103355 | 1472 | 11.38 | 3778 | 0.492 |
| | MAN1A2 | chr1:117984948-117998706 | 427 | 3.69 | 914 | 0.398 |
| | RAF1 | chr3:12641915-12644976 | 1841 | 14.37 | 4800 | 0.577 |
| | ERGIC3 | chr20:34136619-34136916 | 2778 | 62.81 | 7045 | 8.358 |

TABLE 1E

GRCh37 (hg19) - LMI070 Junction2 Metrics

| | Gene ID | J2 Position | J2 NumDatasets | J2 % Data Sets | J2 TotalCounts | J2 % Total counts |
|---|---|---|---|---|---|---|
| Exclusive | SF3B3 | chr16:70561333-70562775 | 1 | 0.01 | 1 | 0.0001 |
| | BENC1 | chr17:40963349-40963672 | 182 | 1.33 | 303 | 0.0333 |
| | GXYLT1 | chr12:42489017-42491243 | 79 | 1.06 | 97 | 0.2236 |
| | SKP1 | chr5:133511077-133512545 | 6 | 0.05 | 6 | 0.0002 |
| | SKP1 | chr5:133511115-133512545 | 13 | 0.10 | 18 | 0.0007 |
| | C12orf4 | chr12:4646681-4647574 | 579 | 5.87 | 1102 | 1.0787 |
| | SSBP1 | chr7:141441260-141441968 | 41 | 0.28 | 60 | 0.0024 |
| | RARS | chr5:167945529-167946085 | 7 | 0.05 | 7 | 0.0008 |
| | RARS | chr5:167945529-167946085 | 7 | 0.05 | 7 | 0.0008 |
| | PDXDC2P | chr16:70065152-70065802 | 1097 | 11.44 | 3160 | 2.6833 |
| | STRADB | chr2:202335835-202337677 | 171 | 1.82 | 260 | 0.1671 |
| | WNK1 | chr12:1004363-1005236 | 33 | 0.26 | 49 | 0.0061 |
| | WDR27 | chr6:170061847-170062399 | 960 | 9.66 | 1533 | 1.1958 |
| | CIP24 | Not Found | Not Found | 0.00 | Not Found | 0.0000 |
| | ITF57 | chr3:107911374-107925474 | 68 | 0.54 | 157 | 0.0269 |
| | HTT | chr4:3215464-3215684 | 738 | 6.62 | 1064 | 0.4371 |
| | SKA2 | Not Found | Not Found | 0.00 | Not Found | 0.0000 |
| | EVC | chr4:5743169-5743442 | 107 | 1.59 | 154 | 0.2071 |
| | DYRK1A | chr21:38794955-38844985 | 11 | 0.10 | 15 | 0.0099 |
| | GNAQ | chr9:80535619-80537076 | 325 | 3.24 | 810 | 0.3614 |
| | ZMYM6 | chr1:35485881-35485983 | 413 | 3.53 | 1465 | 0.7348 |
| | CYB5B | chr16:69482657-69492995 | 16 | 0.12 | 19 | 0.0017 |
| | MMS22L | chr6:97649343-97676769 | 128 | 1.43 | 185 | 0.2029 |
| | MEMO1 | chr2:32112157-32117060 | 1010 | 11.64 | 1589 | 1.6014 |
| | PNISR | chr6:99868461-99873090 | 21 | 0.16 | 29 | 0.0033 |
| Enriched | CACNA2D1 | chr7:81705439-81713748 | 2 | 0.03 | 7 | 0.0073 |
| | SSBP1 | chr7:141441260-141441968 | 41 | 0.28 | 60 | 0.0024 |
| | DDX42 | NA | NA | NA | NA | NA |
| | ASAP1 | chr8:131173040-131179781 | 2397 | 20.94 | 16510 | 4.1600 |
| | DUXAP10 | chr14:19893151-19894699 | 31 | 4.86 | 34 | 2.2636 |
| | AVL9 | chr7:32602526-32609631 | 811 | 7.15 | 1355 | 0.6796 |
| | DYRK1A | chr21:38794955-38844985 | 11 | 0.10 | 15 | 0.0099 |
| | FAM3A | chrX:153741031-153741146 | 1834 | 19.39 | 3467 | 3.5144 |
| | FHOD3 | chr18:34322432-34322699 | 170 | 2.34 | 216 | 0.1906 |
| | TBCA^{∗} | chr5:77070042-77072028 | 1306 | 9.21 | 4838 | 0.5132 |
| | MZT1 | chr13:73299917-73301661 | 51 | 0.41 | 79 | 0.0153 |
| | LINC01296 | chr14:19682353-19683691 | 1 | 0.42 | 1 | 0.3175 |
| | SF3B3 | chr16:70578153-70578340 | 174 | 1.35 | 273 | 0.0310 |
| | SAFB | Not Found | Not Found | 0.00 | Not Found | 0.0000 |
| | GCFC2 | chr2:75929934-75933648 | 534 | 4.76 | 697 | 0.4146 |
| | MRPL45 | chr17:36468625-36474585 | 847 | 6.66 | 1292 | 0.2123 |
| | SPIDR | chr8:48186043-48192449 | 708 | 6.60 | 1131 | 0.7136 |
| | DUXAP8 | chr14:19682353-19683691 | 1 | 0.42 | 1 | 0.3175 |
| | PDXDC1 | chr16:15103419-15103537 | 116 | 0.90 | 190 | 0.0248 |
| | MANIA2 | chr1:117998829-118003110 | 40 | 0.35 | 57 | 0.0248 |
| | RAF1 | chr3:12645037-12645634 | 2187 | 17.08 | 5789 | 0.6955 |
| | ERGIC3 | chr20:34136962-34142142 | 194 | 4.39 | 922 | 1.0939 |

### III. Target Genes for Alternative Splicing Regulation

### A. Inhibitory RNAs

“RNA interference (RNAi)” is the process of sequence-specific, post-transcriptional gene silencing initiated by siRNA. During RNAi, siRNA induces degradation of target mRNA with consequent sequence-specific inhibition of gene expression. Examples of genes whose expression may be inhibited using the expression systems of the present disclosure include, but are not limited to, HTT (for Huntington’s disease), SCA (for Spinocerebellar ataxia (type 1, 2, 3, 6, 7), FXTAS (for Fragile X ataxia syndrome), and FMRP (for Fragile X).

An “inhibitory RNA,” “RNAi,” “small interfering RNA” or “short interfering RNA” or “siRNA” molecule, “short hairpin RNA” or “shRNA” molecule, or “miRNA” is a RNA duplex of nucleotides that is targeted to a nucleic acid sequence of interest. As used herein, the term “siRNA” is a generic term that encompasses the subset of shRNAs and miRNAs. An “RNA duplex” refers to the structure formed by the complementary pairing between two regions of an RNA molecule. siRNA is “targeted” to a gene in that the nucleotide sequence of the duplex portion of the siRNA is complementary to a nucleotide sequence of the targeted gene. In certain embodiments, the siRNAs are targeted to the sequence encoding huntingtin. In some embodiments, the length of the duplex of siRNAs is less than 30 base pairs. In some embodiments, the duplex can be 29, 28, 27, 26, 25, 24, 23, 22, 21, 20, 19, 18, 17, 16, 15, 14, 13, 12, 11 or 10 base pairs in length. In some embodiments, the length of the duplex is 19 to 25 base pairs in length. In certain embodiment, the length of the duplex is 19 or 21 base pairs in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12, 13, 14, 15, 16, 17, 18, 19, 20, 21, 22, 23, 24 or 25 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length.

shRNAs are comprised of stem-loop structures which are designed to contain a 5′ flanking region, siRNA region segments, a loop region, a 3′ siRNA region and a 3′ flanking region. Most RNAi expression strategies have utilized short-hairpin RNAs (shRNAs) driven by strong polIII-based promoters. Many shRNAs have demonstrated effective knock down of the target sequences in vitro as well as in vivo, however, some shRNAs which demonstrated effective knock down of the target gene were also found to have toxicity in vivo.

miRNAs are small cellular RNAs (~22 nt) that are processed from precursor stem loop transcripts. Known miRNA stem loops can be modified to contain RNAi sequences specific for genes of interest. miRNA molecules can be preferable over shRNA molecules because miRNAs are endogenously expressed. Therefore, miRNA molecules are unlikely to induce dsRNA-responsive interferon pathways, they are processed more efficiently than shRNAs, and they have been shown to silence 80% more effectively.

A recently discovered alternative approach is the use of artificial miRNAs (pri-miRNA scaffolds shuttling siRNA sequences) as RNAi vectors. Artificial miRNAs more naturally resemble endogenous RNAi substrates and are more amenable to Pol-II transcription (e.g., allowing tissue-specific expression of RNAi) and polycistronic strategies (e.g., allowing delivery of multiple siRNA sequences). See U.S. Pat. No. 10,093,927, which is incorporated by reference.

The transcriptional unit of a “shRNA” is comprised of sense and antisense sequences connected by a loop of unpaired nucleotides. shRNAs are exported from the nucleus by Exportin-5, and once in the cytoplasm, are processed by Dicer to generate functional siRNAs. “miRNAs” stem-loops are comprised of sense and antisense sequences connected by a loop of unpaired nucleotides typically expressed as part of larger primary transcripts (pri-miRNAs), which are excised by the Drosha-DGCR8 complex generating intermediates known as pre-miRNAs, which are subsequently exported from the nucleus by Exportin-5, and once in the cytoplasm, are processed by Dicer to generate functional siRNAs. “Artificial miRNA” or an “artificial miRNA shuttle vector”, as used herein interchangably, refers to a primary miRNA transcript that has had a region of the duplex stem loop (at least about 9-20 nucleotides) which is excised via Drosha and Dicer processing replaced with the siRNA sequences for the target gene while retaining the structural elements within the stem loop necessary for effective Drosha processing. The term “artificial” arises from the fact the flanking sequences (~35 nucleotides upstream and ~40 nucleotides downstream) arise from restriction enzyme sites within the multiple cloning site of the siRNA. As used herein the term “miRNA” encompasses both the naturally occurring miRNA sequences as well as artificially generated miRNA shuttle vectors.

The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal or a sequence of six Ts.

In designing RNAi there are several factors that need to be considered, such as the nature of the siRNA, the durability of the silencing effect, and the choice of delivery system. To produce an RNAi effect, the siRNA that is introduced into the organism will typically contain exonic sequences. Furthermore, the RNAi process is homology dependent, so the sequences must be carefully selected so as to maximize gene specificity, while minimizing the possibility of cross-interference between homologous, but not gene-specific sequences. Preferably the siRNA exhibits greater than 80%, 85%, 90%, 95%, 98%, or even 100% identity between the sequence of the siRNA and the gene to be inhibited. Sequences less than about 80% identical to the target gene are substantially less effective. Thus, the greater homology between the siRNA and the gene to be inhibited, the less likely expression of unrelated genes will be affected.

In addition, the size of the siRNA is an important consideration. In some embodiments, the present invention relates to siRNA molecules that include at least about 19-25 nucleotides and are able to modulate gene expression. In the context of the present invention, the siRNA is preferably less than 500, 200, 100, 50, or 25 nucleotides in length. More preferably, the siRNA is from about 19 nucleotides to about 25 nucleotides in length.

A siRNA target generally means a polynucleotide comprising a region that encodes a polypeptide, or a polynucleotide region that regulates replication, transcription, or translation or other processes important to expression of the polypeptide, or a polynucleotide comprising both a region that encodes a polypeptide and a region operably linked thereto that regulates expression. Any gene being expressed in a cell can be targeted. Preferably, a target gene is one involved in or associated with the progression of cellular activities important to disease or of particular interest as a research object.

### B. CRISPR Systems

Gene editing is a technology that allows for the modification of target genes within living cells. Recently, harnessing the bacterial immune system of CRISPR to perform on demand gene editing revolutionized the way scientists approach genomic editing. The Cas9 protein of the CRISPR system, which is an RNA guided DNA endonuclease, can be engineered to target new sites with relative ease by altering its guide RNA sequence. This discovery has made sequence specific gene editing functionally effective.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. Examples of genes whose expression may be inhibited or whose sequence may be edited using the CRISPR expression systems of the present disclosure include, but are not limited to, HTT (for Huntington’s disease), SCA (for Spinocerebellar ataxia (type 1, 2, 3, 6, 7)), FXTAS (for Fragile X ataxia syndrome), and FMRP (for Fragile X).

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). CRISPR/Cas systems are classified into two classes, comprising six types and numerous subtypes. The classification is based upon identifying all cas genes in a CRISPR/Cas locus and determining the signature genes in each CRISPR/Cas locus, ultimately determining that the CRISPR/Cas systems can be placed in either Class 1 or Class 2 based upon the genes encoding the effector module, i.e., the proteins involved in the interference stage. Class 1 systems have a multi-subunit crRNA-effector complex, whereas Class 2 systems have a single protein, such as Cas9, Cpf1, C2c1, C2c2, C2c3, or a crRNA-effector complex. Class 1 systems comprise Type I, Type III, and Type IV systems. Class 2 systems comprise Type II, Type V, and Type VI systems. As such, one or more elements of a CRISPR system can derive from any class or type of CRISPR system, e.g., derived from a particular organism comprising an endogenous CRISPR system, such as Streptococcus pyogenes.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor (e.g., KRAB) or activator, to affect gene expression. Alternatively, a CRISPR system with a catalytically inactivate Cas9 further comprises a transcriptional repressor or activator fused to a ribosomal binding protein.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence.” In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. The Cas enzyme may be a target gene under the control of a regulated alternative splicing event, as disclosed herein, either as a chimeric target gene minigene or as a target gene for a chimeric minigene transactivator. The gRNA may be under the control of a constitutive promoter.

Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5- transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### C. Therapeutic Proteins

Some embodiments concern expression of recombinant proteins and polypeptides. Examples of proteins that may be expressed using the expression systems of the present disclosure include, but are not limited to, STXBP1 (also known as Munc18-1; for STXBP1 deficiency, a form neonatal epilepsy, a form of developmental delay), SCN1a (for Dravet syndrome, also known as genetic epileptic encephalopathy, also known as severe myoclonic epilepsy of Infancy (SMEI); mutations in Nav1.1); SCN1b (mutations in Nav1.1 beta subunit); SCN2b (for familial atrial fibrillation; beta 2 subunit of the type II voltage-gated sodium channel); KCNA1 (for dominantly inherited episodic ataxia; muscle spasms with rigidity with or without ataxia); KCNQ2 (KCNQ2-related epilepsies); GABRB3 (early onset epilepsy; β3 subunit of the GABAA receptor); CACNAIA (for familial ataxias and hemiplegic migraines; transmembrane pore-forming subunit of the P/Q-type voltage-gated calcium channel); CHRNA2 (for autosomal dominant nocturnal frontal lobe epilepsy; alpha subunit of the neuronal nicotinic cholinergic receptor (nAChR)); KCNT1 (for autosomal dominant nocturnal frontal lobe epilepsy (ADNFLE) and malignant migrating partial seizures of infancy (MMPSI); sodium-activated potassium channel); SCN8A (for epilepsy and neurodevelopmental disorders; Nav1.6 deficiency, a voltage-dependent sodium channels); CHRNA4-alpha subunit (for autosomal dominant nocturnal frontal lobe epilepsy; mutation in alpha subunit of nicotinic acetylcholine receptor); CHRNB2-b2 subunit (for autosomal dominant nocturnal frontal lobe epilepsy; mutation in alpha subunit of nicotinic acetylcholine receptor); ARX (for Otohara syndrome, polyAla expansion in ARX gene); MECP2 (for Rett syndrome); FMRP (for Fragile X); and CLN3 (for CLN-disease, also known as Juvenile form of Batten’s disease, also known as JNCL). Other examples of therapeutic proteins that may be expressed using the expression systems of the present disclosure include erythropoietin (EPO, for anemia), progranulin (GRN, for neurodegenerative diseases), tripeptidyl-peptidase 1 (TPP1, for lysosomal storage disease), factor IX (F9, for hemophilia), human α-galactosidase (GLA, for Fabry disease), alpha-1-antitrypsin (AIAT, for alpha-1-antitrypsin deficiency), human growth hormone (HGH, for growth hormone deficiency), ion channels, components of the complement pathway, cytokines, chemokines, chemoattractants, protein hormones (e.g. EGF, PDF), protein components of serum, antibodies, secretable toll-like receptors, coagulation factors, kinases growth factors, and other signaling molecules. Other examples of proteins that may be expressed using the expression systems of the present disclosure may be found in Lindy et al. (2018) and Heyne et al. (2018) and U.S. Pat. Publn. 2018/0353616, each of which is incorporated herein by reference in its entirety.

Disorders for which the present invention are useful include, but are not limited to, disorders such as Pompe Disease, Gaucher Disease, beta-thalassemia, Huntington’s Disease; Parkinson’s Disease; muscular dystrophies (such as, e.g. Duchenne and Becker); hemophilia diseases (such as, e.g., hemophilia B (FIX), hemophilia A (FVIII); SMN1-related spinal muscular atrophy (SMA); amyotrophic lateral sclerosis (ALS); GALT-related galactosemia; Cystic Fibrosis (CF); SLC3Al-related disorders including cystinuria; COL4A5-related disorders including Alport syndrome; galactocerebrosidase deficiencies; X-linked adrenoleukodystrophy and adrenomyeloneuropathy; Friedreich’s ataxia; Pelizaeus-Merzbacher disease; TSC1 and TSC2-related tuberous sclerosis; Sanfilippo B syndrome (MPS IIIB); CTNS-related cystinosis; the FMR1-related disorders which include Fragile X syndrome, Fragile X-Associated Tremor/Ataxia Syndrome and Fragile X Premature Ovarian Failure Syndrome; Prader-Willi syndrome; hereditary hemorrhagic telangiectasia (AT); Niemann-Pick disease Type C1; the neuronal ceroid lipofuscinoses-related diseases including Juvenile Neuronal Ceroid Lipofuscinosis (JNCL), Juvenile Batten disease, Santavuori-Haltia disease, Jansky-Bielschowsky disease, and PTT-1 and TPP1 deficiencies; EIF2B1, EIF2B2, EIF2B3, EIF2B4 and EIF2B5-related childhood ataxia with central nervous system hypomyelination/vanishing white matter; CACNAIA and CACNB4-related Episodic Ataxia Type 2; the MECP2-related disorders including Classic Rett Syndrome, MECP2-related Severe Neonatal Encephalopathy and PPM-X Syndrome; CDKL5-related Atypical Rett Syndrome; Kennedy’s disease (SBMA); Notch-3 related cerebral autosomal dominant arteriopathy with subcortical infarcts and leukoencephalopathy (CADASIL); SCNIA and SCN1B-related seizure disorders; the Polymerase G-related disorders which include Alpers-Huttenlocher syndrome, POLG-related sensory ataxic neuropathy, dysarthria, and ophthalmoparesis, and autosomal dominant and recessive progressive external ophthalmoplegia with mitochondrial DNA deletions; X-Linked adrenal hypoplasia; X-linked agammaglobulinemia; Wilson’s disease; and Fabry Disease.

In some aspects, the protein or polypeptide may be modified to increase serum stability. Thus, when the present application refers to the function or activity of “modified protein” or a “modified polypeptide,” one of ordinary skill in the art would understand that this includes, for example, a protein or polypeptide that possesses an additional advantage over the unmodified protein or polypeptide. It is specifically contemplated that embodiments concerning a “modified protein” may be implemented with respect to a “modified polypeptide,” and vice versa.

Recombinant proteins may possess deletions and/or substitutions of amino acids; thus, a protein with a deletion, a protein with a substitution, and a protein with a deletion and a substitution are modified proteins. In some embodiments, these proteins may further include insertions or added amino acids, such as with fusion proteins or proteins with linkers, for example. A “modified deleted protein” lacks one or more residues of the native protein, but may possess the specificity and/or activity of the native protein. A “modified deleted protein” may also have reduced immunogenicity or antigenicity. An example of a modified deleted protein is one that has an amino acid residue deleted from at least one antigenic region that is, a region of the protein determined to be antigenic in a particular organism, such as the type of organism that may be administered the modified protein.

Substitution or replacement variants typically contain the exchange of one amino acid for another at one or more sites within the protein and may be designed to modulate one or more properties of the polypeptide, particularly its effector functions and/or bioavailability. Substitutions may or may not be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine, or methionine; serine to threonine; threonine to serine; tryptophan to tyrosine; tyrosine to tryptophan or phenylalanine; and valine to isoleucine or leucine.

In addition to a deletion or substitution, a modified protein may possess an insertion of residues, which typically involves the addition of at least one residue in the polypeptide. This may include the insertion of a targeting peptide or polypeptide or simply a single residue. Terminal additions, called fusion proteins, are discussed below.

The term “biologically functional equivalent” is well understood in the art and is further defined in detail herein. Accordingly, sequences that have between about 70% and about 80%, or between about 81% and about 90%, or even between about 91% and about 99% of amino acids that are identical or functionally equivalent to the amino acids of a control polypeptide are included, provided the biological activity of the protein is maintained. A recombinant protein may be biologically functionally equivalent to its native counterpart in certain aspects.

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

As used herein, a protein or peptide generally refers, but is not limited to, a protein of greater than about 200 amino acids, up to a full-length sequence translated from a gene; a polypeptide of greater than about 100 amino acids; and/or a peptide of from about 3 to about 100 amino acids. For convenience, the terms “protein,” “polypeptide,” and “peptide are used interchangeably herein.

As used herein, an “amino acid residue” refers to any naturally occurring amino acid, any amino acid derivative, or any amino acid mimic known in the art. In certain embodiments, the residues of the protein or peptide are sequential, without any non-amino acids interrupting the sequence of amino acid residues. In other embodiments, the sequence may comprise one or more non-amino acid moieties. In particular embodiments, the sequence of residues of the protein or peptide may be interrupted by one or more non-amino acid moieties.

Accordingly, the term “protein or peptide” encompasses amino acid sequences comprising at least one of the 20 common amino acids found in naturally occurring proteins, or at least one modified or unusual amino acid.

Certain embodiments of the present invention concern fusion proteins. These molecules may have a therapeutic protein linked at the N- or C-terminus to a heterologous domain. For example, fusions may also employ leader sequences from other species to permit the recombinant expression of a protein in a heterologous host. Another useful fusion includes the addition of a protein affinity tag, such as a serum albumin affinity tag or six histidine residues, or an immunologically active domain, such as an antibody epitope, preferably cleavable, to facilitate purification of the fusion protein. Non-limiting affinity tags include polyhistidine, chitin binding protein (CBP), maltose binding protein (MBP), and glutathione-S-transferase (GST).

Methods of generating fusion proteins are well known to those of skill in the art. Such proteins can be produced, for example, by de novo synthesis of the complete fusion protein, or by attachment of the DNA sequence encoding the heterologous domain, followed by expression of the intact fusion protein.

Production of fusion proteins that recover the functional activities of the parent proteins may be facilitated by connecting genes with a bridging DNA segment encoding a peptide linker that is spliced between the polypeptides connected in tandem. The linker would be of sufficient length to allow proper folding of the resulting fusion protein.

### IV. Splicing Modifiers

A representative splice modifier is LMI070 (5-(1H-Pyrazol-4-yl)-2-(6-((2,2,6,6-tetramethylpiperidin-4-yl)oxy)pyridazin-3-yl)phenol; Spinraza™; Novartis,³¹), which is able to penetrate the blood brain barrier, having the following structure:

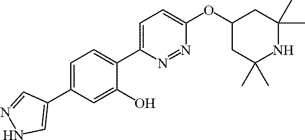 [SMILES: CC1(C)CC(Oc2ccc(-c3ccc(-c4cn[nH]c4)cc3O)nn2)CC(C)(C)N1]

Examples of alternative splicing events where a novel exon is included only in the presence of LMI070, and which can be used for controlling gene expression in the systems of the present disclosure, include, but are not limited to, SF3B3 (chr16:70,526,657-70,529,199), BENC1 (chr17:42,810,759-42,811,797), GXYLT1 (chr12:42,087,786-42,097,614), SKP1 (chr5:134,173,809-134,177,053), C12orf4 (chr12:4,536,017-4,538,508), SSBP1 (chr7:141,739,167-141,742,229), RARS (chr5:168,517,815-168,519,190), PDXDC2P (chr16:70,030,988-70,031,968), STRADB (chr2:201,469,953-201,473,076), WNK1 (chr12:894,562-896,732), WDR27 (chr6:169,660,663-169,662,424), CIP2A (chr3:108,565,355-108,566,638), IFT57 (chr3:108,191,521-108,206,696), WDR27 (chr6:169,660,649-169,662,458), HTT (chr4:3,212,555-3,214,145), SKA2 (chr17:59,112,228-59,119,514), EVC (chr4:5,733,318-5,741,822), DYRKIA (chr21:37,420,144-37,473,056), GNAQ (chr9:77,814,652-77,923,557), ZMYM6 (chr1:35,019,257-35,020,472), CYB5B (chr16:69,448,031-69,459,160), MMS22L (chr6:97,186,342-97,229,533), MEMO1 (chr2:31,883,262-31,892,301), and PNISR (chr6:99,416,278-99,425,413). Examples of alternative splicing events where the inclusion of a novel exon is enhanced by the presence of LMI070, and which can be used for controlling gene expression in the systems of the present disclosure, include, but are not limited to, CACNA2D1 (chr7:82,066,406-82,084,958), SSBP1 (chr7:141,739,083-141,742,248), DDX42 (chr17:63,805,048-63,806,672), ASAP1 (chr8:130,159,817-130,167,688), DUXAP10 (chr14:19,294,564-19,307,199), AVL9 (chr7:32,558,783-32,570,372), DYRK1A (chr21:37,419,920-37,472,960), FAM3A (chrX:154,512,311-154,512,939), FHOD3 (chr18:36,740,620-36,742,886), TBCA (chr5:77,707,994-77,777,000), MZT1 (chr13:72,718,939-72,727,611), LINC01296 (chr14:19,092,877-19,096,652), SF3B3 (chr16:70,541,627-70,544,553), SAFB (chr19:5,654,060-5,654,457), GCFC2 (chr2:75,702,163-75,706,652), MRPL45 (chr17:38,306,450-38,319,088), SPIDR (chr8:47,260,788-47,280,196), DUXAP8 (chr22:15,815,315-15,828,713), PDXDC1 (chr16:15,008,772-15,009,763), MANIA2 (chr1:117,442,104-117,461,030), RAF1 (chr3:12,600,376-12,604,350), and ERGIC3 (chr20:35,548,787-35,554,452). For the above lists, each genomic location includes the upstream and downstream exon and the intervening intronic sequence targeted by LMI070.

Analogues of splice modifiers such as LMI070 that can be used also are included, for example, 6-(naphthalen-2-yl)-N-(2,2,6,6-tetramethylpiperidin-4-yl)pyridazin-3-amine; 6-(benzo[b]thio-phen-2-yl)-N-methyl-N-(2,2,6,6-tetra-methylpiperidin-4-yl)pyridazin-3-amine; 2-(6-(2,2,6,6-tetramethylpiperidin-4-ylamino)-pyridazin-3-yl)phenol; 2-(6-(methyl-(2,2,6,6-tetra-methylpiperidin-4-yl)amino)pyridazin-3-yl)benzo[b]-thiophene-5-carbonitrile; 6-(quinolin-3-yl)-N-(2,2,6,6-tetramethyl-piperidin-4-yl)pyridazin-3-amine; 3-(benzo[b]-thiophen-2-yl)-6-(2,2,6,6-tetra-methylpiperidin-4-yloxy)pyridazine; 2-(6-(methyl-(2,2,6,6-tetra-methylpiperidin-4-yl)amino)-pyridazin-3-yl)phenol; 6-(6-(methyl-(2,2,6,6-tetra-methylpiperidin-4-yl)amino)-pyridazin-3-yl)naphthalen-2-ol; 6-(benzo[b]-thiophen-2-yl)-N-(2,2,6,6-tetra-methylpiperidin-4-yl)pyridazin-3-amine; 7-(6-((2,2,6,6-tetramethylpiperidin-4-yl)oxy)pyridazin-3-yl)isoquinoline; 6-(6-((2,2,6,6-tetramethylpiperidin-4-yl)oxy)pyridazin-3-yl)isoquinoline; N-methyl-6-(quinolin-7-yl)-N-(2,2,6,6-tetramethyl-piperidin-4-yl)pyridazin-3-amine; N-methyl-6-(quinolin-6-yl)-N-(2,2,6,6-tetramethylpiperidin-4-yl)pyridazin-3-amine; 6-(isoquinolin-7-yl)-N-methyl-N-(2,2,6,6-tetramethylpiperidin-4-yl)pyridazin-3-amine; 6-(isoquinolin-6-yl)-N-methyl-N-(2,2,6,6-tetramethylpiperidin-4-yl)pyridazin-3-amine; 6-(imidazo[1,2-a]pyridin-6-yl-pyridazin-3-yl)-methyl-(2,2,6,6-tetramethyl-piperidin-4-yl)-amine; methyl-[6-(6-phenyl-pyridin-3-yl)-pyridazin-3-yl]-(2,2,6,6-tetramethyl-piperidin-4-yl)-amine; methyl-[6-(6-pyrrol-1-yl-pyridin-3-yl)-pyridazin-3-yl]-(2,2,6,6-tetramethyl-piperidin-4-yl)-amine; methyl-[6-(6-pyrazol-1-yl-pyridin-3-yl)-pyridazin-3-yl]-(2,2,6,6-tetramethyl-piperidin-4-yl)-amine; methyl-(6-quinoxalin-2-yl-pyridazin-3 -yl)-(2,2, 6, 6-tetramethyl-piperidin-4-yl)-amine; methyl-(6-quinolin-3-yl-pyridazin-3-yl)-(2,2,6,6-tetramethyl-piperidin-4-yl)-amine; N-methyl-6-(phthalazin-6-yl)-N-(2,2,6,6-tetramethylpiperidin-4-yl)pyridazin-3-amine; 6-(benzo[c][1,2,5]oxa-diazol-5-yl)-N-(2,2,6,6-tetramethyl-piperidin-4-yl)pyridazin-3-amine; 6-(benzo[d]thiazol-5-yl)-N-(2,2,6,6-tetramethyl-piperidin-4-yl)pyridazin-3-amine; 6-(2-methylbenzo-[d]oxazol-6-yl)-N-(2,2,6,6-tetramethyl-piperidin-4-yl)pyridazin-3-amine; 3-(6-(methyl(2,2,6,6-tetramethylpiperidin-4-yl)amino)pyridazin-3-yl)naphthalen-2-ol; 5-chloro-2-(6-(methyl(1,2,2,6,6-pentamethylpiperidin-4-yl)amino)pyridazin-3-yl)phenol; 3-(6-(2,2,6,6-tetramethylpiperidin-4-ylamino)pyridazin-3-yl)naphthalen-2-ol; 5-chloro-2-(6-(1,2,2,6,6-pentamethylpiperidin-4-ylamino)pyridazin-3-yl)phenol; 4-hydroxy-3-(6-(methyl(2,2,6,6-tetramethylpiperidin-4-yl)amino)pyridazin-3-yl)benzonitrile; 3-[6-(2,2,6,6-tetramethyl-piperidin-4-yloxy)-pyridazin-3-yl]-naphthalen-2-ol; 2-{6-[methyl-(2,2,6,6-tetramethyl-piperidin-4-yl)-amino]-pyridazin-3-yl}-4-trifluoromethyl-phenol; 2-fluoro-6-{6-[methyl-(2,2,6,6-tetramethyl-piperidin-4-yl)-amino] -pyridazin-3-yl} -phenol; 3,5-dimethoxy-2-{6-[methyl-(2,2,6,6-tetramethyl-piperidin-4-yl)-amino]-pyridazin-3-yl}-phenol; 4,5-dimethoxy-2-{6-[methyl-(2,2,6,6-tetramethyl-piperidin-4-yl)-amino]-pyridazin-3-yl}-phenol; 5-methoxy-2-{6-[methyl-(2,2,6,6-tetramethyl-piperidin-4-yl)-amino]pyridazin-3-yl}-phenol; 4,5-difluoro-2-{6-[methyl-(2,2,6,6-tetramethyl-piperidin-4-yl)-amino]-pyridazin-3-yl}-phenol; 5-fluoro-2-{6-[methyl-(2,2,6,6-tetramethyl-piperidin-4-yl)-amino]-pyridazin-3-yl}-phenol; 3-hydroxy-4-(6-(methyl(2,2,6,6-tetramethylpiperidin-4-yl)amino)pyridazin-3-yl)benzonitrile; 1-allyl-6-(6-(methyl(2,2,6,6-tetramethylpiperidin-4-yl)amino)pyridazin-3-yl)naphthalen-2-ol; 6-(benzo[b]thiophen-2-yl)-N-(1,2,2,6,6-pentamethylpiperidin-4-yl)pyridazin-3-amine; N-allyl-3-hydroxy-4-(6-(methyl(2,2,6,6-tetramethylpiperidin-4-yl)amino)pyridazin-3-yl)benzamide; 2-(6-(methyl (2,2,6,6-tetramethylpiperidin-4-yl)amino)pyridazin-3-yl)-5-(1H-pyrazol-1-yl)phenol; 5-(5-methyl-oxazol-2-yl)-2-{6-[methyl-(2,2,6,6-tetramethyl-piperidin-4-yl)-amino ]pyridazin-3-y I} -phenol; 5-(4-hydroxymethyl)-1H-pyrazole-1-yl)-2-(6-(methyl(2,2,6,6-tetramethylpiperidin-4-yl)amino)pyridazin-3-yl)phenol; 5-(1H-imidazole-1-yl)-2-(6-(methyl(2,2,6,6-tetramethyl-piperidin-4-yl)amino)pyridazin-3-yl)phenol; 5-(4-amino-1H-pyrazole-1-yl)-2-(6-(methyl(2,2,6,6-tetramethylpiperidin-4-yl)amino)pyridazin-3-yl)phenol; 5-(4-amino-1H-pyrazol-1-yl)-2-(6-(methyl(2,2,6,6-tetramethylpiperidin-4-yl)amino)pyridazin-3-yl)phenol; 5-(3-amino-pyrazol-1-yl)-2-{6-[methyl-(2,2,6,6-tetramethyl-piperidin-4-yl)-amino]pyridazin-3-yl} -phenol; 2-(6-(methyl(2,2,6,6-tetramethylpiperidin-4-yl)amino)pyridazin-3-yl)-5-(1-(2-morpholino-ethyl)-1H-pyrazol-4-yl)phenol; 2-(6-(methyl (2,2,6,6-tetramethylpiperidin-4-yl)amino)pyridazin-3-yl)-5-(1-methyl-1H-pyrazol-4-yl)phenol; 5-(5-amino-1H-pyrazol-1-yl)-2-(6-(methyl-(2,2,6,6-tetramethyl-piperidin-4-yl)amino)pyridazin-3-yl)phenol; 2-(6-(methyl (2,2,6,6-tetramethylpiperidin-4-yl)amino)pyridazin-3-yl)-4-(1H-pyrazol-1-yl)phenol; 2-{6-[(2-hydroxy-ethyl)-(2,2,6,6-tetramethyl-piperidin-4-yl)-amino|pyridazin-3-yl } -5-pyrazol-1-yl-phenol; 2-(6-(piperidin-4-yloxy)pyridazin-3-yl)-5-(1H-pyrazol-1-yl)phenol; 2-(6-(((2S,4R,6R)-2,6-dimethylpiperidin-4-yl)oxy)pyridazin-3-yl)-5-(1H-pyrazol-1-yl)phenol; 2-(6-((-2,6-di methyl piperidin-4-yl)oxy)pyridazin-3-yl)-5-(1H-pyrazol-1-yl)phenol; 2-(6-((-2,6-di methyl piperidin-4-yl)oxy)pyridazin-3-yl)-5-(1H-pyrazol-1-yl)phenol; 5-(1H-pyrazol-1-yl)-2-(6-(pyrrolidin-3-yloxy)pyridazin-3-yl)phenol; 2-(6-((-2-methylpiperidin-4-yl)oxy)pyridazin-3-yl)-5-(1H-pyrazol-1-yl)phenol; (S)-5-(1H-Pyrazol-1-yl)-2-(6-(pyrrolidin-3-ylmethoxy)pyridazin-3-yl)phenol; (R)-5-(1H-pyrazol-1-yl)-2-(6-(pyrrolidin-3-ylmethoxy)pyridazin-3-yl)phenol; 2-(6-((3-fluoropiperidin-4-yl)oxy)pyridazin-3-yl)-5-(1H-pyrazol-1-yl)-phenol; 2-[6-(1,2,2,6,6-pentamethyl-piperidin-4-yloxy)-pyridazin-3-yl]-5-pyrazol-1-yl-phenol; 5-pyrazol-1-yl-2-[6-(2,2,6,6-tetramethyl-piperidin-4-yloxy)-pyridazin-3-yl]-phenol; 5-(1H-Pyrazol-4-yl)-2-(6-((2,2,6,6-tetramethylpiperidin-4-yl)oxy)pyridazin-3-yl)phenol; 2-(6-piperazin-1-yl-pyridazin-3-yl)-5-pyrazol-1-yl-phenol; 3-[6-(azetidin-3-ylamino)-pyridazin-3-yl]-naphthalen-2-ol; 2-[6-(azetidin-3-ylamino)-pyridazin-3-yl]-5-pyrazol-1-yl-phenol; 2-[6-(3,5-di methyl-piperazin-1-yl)-pyridazin-3-yl]-5-pyrazol-1-yl-phenol; 2-[6-(7-methyl-2,7-diaza-spiro[4.4]non-2-yl)-pyridazin-3-yl]-5-pyrazol-1-yl-phenol; 2-(6-[1,4]diazepan-1-yl-pyridazin-3-yl)-5-pyrazol-1-yl-phenol; 2-{6-[4-(2-hydroxy-ethyl)-piperazin-1-yl]-pyridazin-3-yl}-5-pyrazol-1-yl-phenol; 2-[6-(3,6-diaza-bicyclo[3.2.1]oct-3-yl)-pyridazin-3-yl]-5-pyrazol-1-yl-phenol; 2-[6-(2,7-diaza-spiro[3.5]non-7-yl)-pyridazin-3-yl]-5-pyrazol-1-yl-phenol; 2-[6-(3-hydroxy-methyl-piperazin-1-yl)-pyridazin-3-yl]-5-pyrazol-1-yl-phenol; 2-[6-(1,7-diaza-spiro[4.4]non-7-yl)-pyridazin-3-yl]-5-pyrazol-1-yl-phenol; 2-[6-(4-amino-4-methyl-piperidin-1-yl)-pyridazin-3-yl]-5-pyrazol-1-yl-phenol; 2-[6-(3-dimethyl-amino-piperidin-1-yl)-pyridazin-3-yl]-5-pyrazol-1-yl-phenol; 2-[6-(1,2,2,6,6-pentamethyl-piperidin-4-ylamino)-pyridazin-3-yl]-5-pyrazol-1-yl-phenol; 2-[6-(3,3-di methyl-piperazin-1-yl)-pyridazin-3-yl]-5-pyrazol-1-yl-phenol; 2-(6-(7-(2-hydroxyethyl)-2,7-diazaspiro[4.4]-nonan-2-yl)pyridazin-3-yl)-5-(1H-pyrazol-1-yl)phenol; 2-(6-((3aR,6aS)-hexahydropyrrolo[3,4-c]pyrrol-2(1H)-yl)pyridazin-3-yl)-5-(1H-pyrazol-1-yl)phenol; 3-(6-(piperazin-1-yl)pyridazin-3-yl)naphthalene-2,7-diol; 5-pyrazol-1-yl-2-[6-(1,2,3,6-tetrahydro-pyridin-4-yl)-pyridazin-3-yl]-phenol; 2-(6-piperidin-4-yl-pyridazin-3-yl)-5-pyrazol-1-yl-phenol; 3-(6-(1,2,3,6-tetra-hydropyridin-4-yl)pyridazin-3-yl)naphthalen-2-ol; 3-(6-(1,2,3,6-tetrahydropyridin-4-yl)pyridazin-3-yl)naphthalene-2,7-diol; 3-(6-(2,2,6,6-tetramethyl-1,2,3,6-tetrahydropyridin-4-yl)pyridazin-3-yl)naphthalene-2,7-diol; 3-(6-(1-methyl-1,2,3,6-tetrahydropyridin-4-yl)pyridazin-3-yl)naphthalene-2,7-diol; 3-(6-(piperidin-4-yl)pyridazin-3-yl)naphthalene-2,7-diol; 3-(6-((2,2,6,6-tetramethylpiperidin-4-yl)oxy)pyridazin-3-yl)naphthalene-2,7-diol; 3-(6-(methyl(2,2,6,6-tetramethylpiperidin-4-yl)amino)pyridazin-3-yl)naphthalene-2,7-diol; 3-(6-((2,2,6,6-tetramethylpiperidin-4-yl)amino)pyridazin-3-yl)naphthalene-2,7-diol; [3-(7-hydroxy-6-{6-[methyl-(2,2,6,6-tetramethyl-piperidin-4-yl)-amino]-pyridazin-3-yl}-naphthalen-2-yloxy)-propyl]-carbamic acid tert-butyl ester; 7-(3-amino-propoxy)-3-{6-[methyl-(2,2,6,6-tetramethyl-piperidin-4-yl)-amino]-pyridazin-3-yl}-naphthalen-2-ol; N-[3-(7-hydroxy-6-{6[methyl-(2,2,6,6-tetramethyl-piperidin-4-yl)-amino]-pyridazin-3-yl}-naphthalen-2-yloxy)-propyl]-acetamide; 7-(3-hydroxypropoxy)-3-(6-(methyl(2,2,6,6-tetramethylpiperidin-4-yl)amino)pyridazin-3-yl)naphthalen-2-ol; 7-(3-methoxypropoxy)-3-(6-(methyl(2,2,6,6-tetramethylpiperidin-4-yl)amino)pyridazin-3-yl)naphthalen-2-ol; 7-(2-morpholinoethoxy)-3-(6-((2,2,6,6-tetramethylpiperidin-4-yl)oxy)pyridazin-3-yl)naphthalen-2-ol; 3-(6-(piperidin-4-ylmethyl)pyridazin-3-yl)naphthalen-2-ol; 5-(1H-pyrazol-1-yl)-2-(6-((2,2,6,6-tetramethylpiperidin-4-yl)methyl)pyridazin-3-yl)phenol; 3-methoxy-2-(6-(methyl (2,2,6-trimethylpiperidin-4-yl)amino)pyridazin-3-yl)-5-(5-methyloxazol-2-yl)phenol; 2-(6-((6S)-6-((S)-1-hydroxyethyl)-2,2-dimethylpiperidin-4-yloxy)pyridazin-3-yl)-5-(1H-pyrazol-1-yl)phenol; 7-hydroxy-6-(6-(methyl(2,2,6,6-tetramethylpiperidin-4-yl)amino)pyridazin-3-yl)-2-naphthonitrile; 3-(6-(methyl (2,2,6,6-tetramethylpiperidin-4-yl)amino)pyridazin-3-yl)-7-(piperidin-1-ylmethyl)naphthalen-2-ol; 3-(6-(methyl (2,2,6,6-tetramethylpiperidin-4-yl)amino)pyridazin-3-yl)-7-(pyrrolidin-1-ylmethyl)naphthalen-2-ol; 1-bromo-6-(6-(methyl(2,2,6,6-tetramethylpiperidin-4-yl)amino)pyridazin-3-yl)naphthalene-2,7-diol; 1-chloro-6-(6-(methyl(2,2,6,6-tetramethylpiperidin-4-yl)amino)pyridazin-3-yl)naphthalene-2,7-diol; 7-methoxy-3-(6-(methyl(2,2,6,6-tetramethylpiperidin-4-yl)amino)pyridazin-3-yl)naphthalen-2-ol; 7-methoxy-3-(6-(methyl(1,2,2,6,6-pentamethylpiperidin-4-yl)amino)pyridazin-3-yl)naphthalen-2-ol; 7-(3,6-dihydro-2H-pyran-4-yl)-3-(6-(methyl(2,2,6,6-tetramethylpiperidin-4-yl)amino)pyridazin-3-yl)naphthalen-2-ol; 3-(6-(methyl(2,2,6,6-tetramethylpiperidin-4-yl)amino)pyridazin-3-yl)-7-(tetrahy dro-2H-pyran-4-yl)naphthalen-2-ol; 7-(difluoromethyl)-3-(6-(methyl(2,2,6,6-tetramethylpiperidin-4-yl)amino)pyridazin-3-yl)naphthalen-2-ol; 7-((4-hydroxy-2-methylbutan-2-yl)oxy)-3-(6-(methyl(2,2,6,6-tetramethylpiperidin-4-yl)amino)pyridazin-3-yl)naphthalen-2-ol; 7-(3-hydroxy-3-methylbutoxy)-3-(6-(methyl(2,2,6,6-tetramethylpiperidin-4-yl)amino)pyridazin-3-yl)naphthalen-2-ol; 2-(6-(methyl(2,2,6,6-tetramethylpiperidin-4-yl)amino)pyridazin-3-yl)-5-(1H-pyrazol-4-yl)benzene-1,3-diol; 3-methoxy-2-(6-(methyl(2,2,6,6-tetramethylpiperidin-4-yl)amino)pyridazin-3-yl)-5-(1H-pyrazol-4-yl)phenol; 5-(1H-pyrazol-4-yl)-2-(6-((2,2,6,6-tetramethylpiperidin-4-yl)amino)pyridazin-3-yl)-3-(trifluoromethoxy)phenol; 2-(6-(methyl(2,2,6,6-tetramethylpiperidin-4-yl)amino)pyridazin-3-yl)-5-(1-methyl-1H-pyrazol-4-yl)-3-(trifluoromethoxy)phenol; 2-(6-(methyl(2,2,6,6-tetramethylpiperidin-4-yl)amino)pyridazin-3-yl)-5-(1H-pyrazol-4-yl)-3-(trifluoromethoxy)phenol; 4-(3-hydroxy-4-(6-(methyl(2,2,6,6-tetramethylpiperidin-4-yl)amino)pyridazin-3-yl)-5-(trifluoromethoxy)phenyl)-1-methylpyridin-2(1H)-one; 3-methoxy-2-(6-(methyl(2,2,6,6-tetramethylpiperidin-4-yl)amino)pyridazin-3-yl)-5-(1-methyl-1H-pyrazol-4-yl)phenol; 3-methoxy-2-(6-(methyl(2,2,6,6-tetramethylpiperidin-4-yl)amino)pyridazin-3-yl)-5-(5,6,7,8-tetrahydroimidazo[1,2-a]pyridin-3-yl)phenol; 3-methoxy-2-(6-(methyl(2,2,6,6-tetramethylpiperidin-4-yl)amino)pyridazin-3-yl)-5-(pyridin-3-yl)phenol; 5-(1-cyclopentyl-1H-pyrazol-4-yl)-3-methoxy-2-(6-(methyl(2,2,6,6-tetramethylpiperidin-4-yl)amino)pyridazin-3-yl)phenol; 3′,5-dimethoxy-4-(6-(methyl(2,2,6,6-tetramethylpiperidin-4-yl)amino)pyridazin-3-yl)-[1,1′-biphenyl]-3-ol; 3-(benzyloxy)-2-(6-(methyl(2,2,6,6-tetramethylpiperidin-4-yl)amino)pyridazin-3-yl)-5-(5-methyloxazol-2-yl)phenol; 3-ethoxy-2-(6-(methyl(2,2,6,6-tetramethylpiperidin-4-yl)amino)pyridazin-3-yl)-5-(5-methyloxazol-2-yl)phenol; 3-(cyclopropylmethoxy)-2-(6-(methyl(2,2,6,6-tetramethylpiperidin-4-yl)amino)-pyridazin-3-yl)-5-(5-methyloxazol-2-yl)phenol; 2-methyl-5-(6-(methyl(2,2,6,6-tetramethylpiperidin-4-yl)amino)pyridazin-3-yl)-1H-benzo[d]imidazol-6-ol; 5-chloro-2-(6-(methyl(2,2,6,6-tetramethylpiperidin-4-yl)amino)pyridazin-3-yl)phenol; 5-(1H-pyrazol-1-yl)-2-(6-((2,2,6,6-tetramethylpiperidin-4-yl)amino)pyridazin-3-yl)phenol; 3-hydroxy-4-(6-((2,2,6,6-tetramethylpiperidin-4-yl)amino)pyridazin-3-yl)benzonitrile; 2-(6-((2,2-dimethylpiperidin-4-yl)oxy)pyridazin-3-yl)-5-(1H-pyrazol-1-yl)phenol; 2-(6-(methyl(2,2,6,6-tetramethylpiperidin-4-yl)amino)pyridazin-3-yl)-4-(1H-pyrazol-4-yl)phenol; 2-(6-(methyl(2,2,6,6-tetramethylpiperidin-4-yl)amino)pyridazin-3-yl)-4-(4,5,6,7-tetrahydropyrazolo[1,5-a]pyridin-3-yl)phenol; 2-(6-(methyl(2,2,6,6-tetramethylpiperidin-4-yl)amino)pyridazin-3-yl)-4-(4,5,6,7-tetrahydropyrazolo[1,5-a]pyrazin-3-yl)phenol; 4-(1H-indol-2-yl)-2-(6-(methyl(2,2,6,6-tetramethylpiperidin-4-yl)amino)pyridazin-3-yl)phenol; 4-(cyclopent-1-en-1-yl)-2-(6-(methyl(2,2,6,6-tetramethylpiperidin-4-yl)amino)pyridazin-3-yl)phenol; 2-(6-(methyl(2,2,6,6-tetramethylpiperidin-4-yl)amino)pyridazin-3-yl)-4-(1H-pyrazol-3-yl)phenol; 4-(4-hydroxy-3-(6-(methyl(2,2,6,6-tetramethylpiperidin-4-yl)amino)pyridazin-3-yl)phenyl)pyridin-2-ol; 4-(4-hydroxy-3-(6-((2,2,6,6-tetramethylpiperidin-4-yl)oxy)pyridazin-3-yl)phenyl)-1-methylpyridin-2(1H)-one; 4-(4-hydroxy-3-(6-((2,2,6,6-tetramethylpiperidin-4-yl)oxy)pyridazin-3-yl)phenyl)pyridin-2-ol; 5-(1H-indazol-7-yl)-2-(6-(methyl(2,2,6,6-tetramethylpiperidin-4-yl)amino)pyridazin-3-yl)phenol; 4-chloro-2-(6-(methyl(2,2,6,6-tetramethylpiperidin-4-yl)amino)pyridazin-3-yl)-5-(1H-pyrazol-4-yl)phenol; 4-fluoro-2-(6-(methyl(2,2,6,6-tetramethylpiperidin-4-yl)amino)pyridazin-3-yl)-5-(1H-pyrazol-4-yl)phenol; 5-fluoro-4-(1H-imidazol-4-yl)-2-(6-(methyl(2,2,6,6-tetramethylpiperidin-4-yl)amino)pyridazin-3-yl)phenol; 5-fluoro-2-(6-(methyl(2,2,6,6-tetramethylpiperidin-4-yl)amino)pyridazin-3-yl)-4-(1H-pyrazol-4-yl)phenol; 5-fluoro-2-(6-(methyl(2,2,6,6-tetramethylpiperidin-4-yl)amino)pyridazin-3-yl)-4-(1H-pyrazol-5-yl)phenol; 6-hydroxy-5-(6-(methyl(2,2,6,6-tetramethylpiperidin-4-yl)amino)pyridazin-3-yl)-2,3-dihydro-1H-inden-1-one; 6-(6-(methyl(2,2,6,6-tetramethylpiperidin-4-yl)amino)pyridazin-3-yl)-1,4-dihydroindeno| 1,2-c]pyrazol-7-ol; 6-hy droxy-5-(6-(methyl(2,2,6,6-tetramethylpiperidin-4-yl)amino)pyridazin-3-yl)-2,3-dihydro-1H-inden-1-one oxime hydrochloride salt; 5-(6-(methyl(2,2,6,6-tetramethylpiperidin-4-yl)amino)pyridazin-3-yl)-2,3-dihydro-1H-indene-1,6-diol; 2-amino-6-(6-(methyl(2,2,6,6-tetramethylpiperidin-4-yl)amino)pyridazin-3-yl)-8H-indeno[1,2-d]thiazol-5-ol hydrochloride salt; 9-(6-(methyl(2,2,6,6-tetramethylpiperidin-4-yl)amino)pyridazin-3-yl)-5,6-dihydroimidazo[5,1-a]isoquinolin-8-ol hydrochloride salt; 4-hydroxy-3-(6-(methyl(2,2,6,6-tetramethylpiperidin-4-yl)amino)pyridazin-3-yl)-N-((1-methyl-1H-pyrazol-4-yl)methyl)benzamide; 4-(4-(hydroxymethyl)-1H-pyrazol-1-yl)-2-(6-(methyl(2,2,6,6-tetramethylpiperidin-4-yl)amino)pyridazin-3-yl)phenol; 5-(1H-pyrazol-4-yl)-2-(6-((2,2,6,6-tetramethylpiperidin-4-yl)methyl)pyridazin-3-yl)phenol; 6-(3-(benzyloxy)isoquinolin-6-yl)-N-methyl-N-(2,2,6,6-tetramethylpiperidin-4-yl)pyridazin-3-amine; 6-(1-(benzyloxy)isoquinolin-7-yl)-N-methyl-N-(2,2,6,6-tetramethylpiperidin-4-yl)pyridazin-3-amine; 3-fluoro-5-(2-methoxypyridin-4-yl)-2-(6-(methyl(2,2,6,6-tetramethylpiperidin-4-yl)amino)pyridazin-3-yl)phenol hydrochloride salt; 4-(3-fluoro-5-hydroxy-4-(6-(methyl(2,2,6,6-tetramethylpiperidin-4-yl)amino)pyridazin-3-yl)phenyl)pyridin-2(1H)-one hydrochloride salt; 4-(3-fluoro-5-hydroxy-4-(6-(methyl(2,2,6,6-tetramethylpiperidin-4-yl)amino)pyridazin-3-yl)phenyl)-1-methylpyridin-2(1H)-one hydrochloride salt; 5-(3-fluoro-5-hydroxy-4-(6-(methyl(2,2,6,6-tetramethylpiperidin-4-yl)amino)pyridazin-3-yl)phenyl)-1-methylpyridin-2(1H)-one hydrochloride salt; 3-fluoro-5-(1H-pyrazol-4-yl)-2-(6-((2,2,6,6-tetramethylpiperidin-4-yl)oxy)pyridazin-3-yl)phenol hydrochloride salt; 5-chloro-3-fluoro-2-(6-(methyl(2,2,6,6-tetramethylpiperidin-4-yl)amino)pyridazin-3-yl)phenol hydrochloride salt; 3-fluoro-2-(6-(methyl(2,2,6,6-tetramethylpiperidin-4-yl)amino)pyridazin-3-yl)-5-(1H-pyrazol-4-yl)phenol hydrochloride salt; 3-fluoro-2-(6-(methyl(2,2,6,6-tetramethylpiperidin-4-yl)amino)pyridazin-3-yl)-5-(1-methyl-1H-pyrazol-4-yl)phenol hydrochloride salt; 5-(5-methoxypyridin-3-yl)-2-(6-(methyl(2,2,6,6-tetramethylpiperidin-4-yl)amino)pyridazin-3-yl)phenol; 5-(3-hydroxy-4-(6-methyl(2,2,6,6-tetramethylpiperidin-4-yl)amino)pyridazin-3-yl)phenyl)pyridin-2-ol; 4-(3-hydroxy-4-(6-methyl(2,2,6,6-tetramethylpiperidin-4-yl)amino)pyridazin-3-yl)phenyl)pyridin-2-ol; 5-(6-methoxypyridin-3-yl)-2-(6-(methyl(2,2,6,6-tetramethylpiperidin-4-yl)amino)pyridazin-3-yl)phenol; 5-(3-hydroxy-4-(6-(methyl(2,2,6,6-tetramethylpiperidin-4-yl)amino)pyridazin-3-yl)phenyl)-3-(trifluoromethyl)pyridin-2-ol; 5-(3-hydroxy-4-(6-(methyl(2,2,6,6-tetramethylpiperidin-4-yl)amino)pyridazin-3-yl)phenyl)-1-methylpyridin-2(1H)-one; 4-(3-hydroxy-4-(6-(methyl(2,2,6,6-tetramethylpiperidin-4-yl)amino)pyridazin-3-yl)phenyl)-1-methylpyridin-2(1H)-one; 5-(2-methoxypyridin-4-yl)-2-(6-(methyl(2,2,6,6-tetramethylpiperidin-4-yl)amino)pyridazin-3-yl)phenol; 4-(3-hydroxy-4-(6-((2,2,6,6-tetramethylpiperidin-4-yl)oxy)pyridazin-3-yl)phenyl)pyridin-2-ol; 5-(6-(dimethylamino)pyridin-3-yl)-2-(6-(methyl(2,2,6,6-tetramethylpiperidin-4-yl)amino)pyridazin-3-yl)phenol; 4-(3-hydroxy-4-(6-((2,2,6,6-tetramethylpiperidin-4-yl)oxy)pyridazin-3-yl)phenyl)-1-methylpyridin-2(1H)-one; 2-(6-(methyl(2,2,6,6-tetramethylpiperidin-4-yl)amino)pyridazin-3-yl)-5-(pyrimidin-5-yl)phenol; 5-(3-hydroxy-4-(6-(methyl(2,2,6,6-tetramethylpiperidin-4-yl)amino)pyridazin-3-yl)phenyl)pyridin-3-ol; 1-cyclopropyl-4-(3-hydroxy-4-(6-(methyl(2,2,6,6-tetramethylpiperidin-4-yl)amino)pyridazin-3-yl)phenyl)pyridin-2(1H)-one; 2-(6-(methyl(2,2,6,6-tetramethylpiperidin-4-yl)amino)pyridazin-3-yl)-5-(1,2,3,6-tetrahydropyridin-4-yl)phenol; 5-(cyclopent-1-en-1-yl)-2-(6-(methyl(2,2,6,6-tetramethylpiperidin-4-yl)amino)pyridazin-3-yl)phenol; 5-(3,6-dihydro-2H-pyran-4-yl)-2-(6-(methyl(2,2,6,6-tetramethylpiperidin-4-yl)amino)pyridazin-3-yl)phenol; 5-(imidazo[1,5-a]pyridin-7-yl)-2-(6-(methyl(2,2,6,6-tetramethylpiperidin-4-yl)amino)pyridazin-3-yl)phenol; 5-(imidazo[1,2-a]pyridin-7-yl)-2-(6-(methyl(2,2,6,6-tetramethylpiperidin-4-yl)amino)pyridazin-3-yl)phenol; 2-(6-(methyl(2,2,6,6-tetramethylpiperidin-4-yl)amino)pyridazin-3-yl)-5-(2-methylpyridin-4-yl)phenol; 5-(1H-imidazol-2-yl)-2-(6-(methyl(2,2,6,6-tetramethylpiperidin-4-yl)amino)pyridazin-3-yl)phenol; 5-(1H-imidazol-4-yl)-2-(6-(methyl(2,2,6,6-tetramethylpiperidin-4-yl)amino)pyridazin-3-yl)phenol; 5-(imidazo[1,2-a]pyrazin-3-yl)-2-(6-(methyl(2,2,6,6-tetramethylpiperidin-4-yl)amino)pyridazin-3-yl)phenol; 2-(6-(methyl(2,2,6,6-tetramethylpiperidin-4-yl)amino)pyridazin-3-yl)-5-(5,6,7,8-tetrahydroimidazo[1,2-a]pyrazin-3-yl)phenol; 2-(6-(methyl(2,2,6,6-tetramethylpiperidin-4-yl)amino)pyridazin-3-yl)-5-(4-methyl-1H-imidazol-2-yl)phenol; 2-(6-(methyl(2,2,6,6-tetramethylpiperidin-4-yl)amino)pyridazin-3-yl)-5-(1-methyl-1H-imidazol-4-yl)phenol; 2-(6-(methyl(2,2,6,6-tetramethylpiperidin-4-yl)amino)pyridazin-3-yl)-5-(1-methyl-1H-imidazol-5-yl)phenol; 2-(6-(methyl(2,2,6,6-tetramethylpiperidin-4-yl)amino)pyridazin-3-yl)-5-(4-nitro-1H-imidazol-2-yl)phenol; 2-(6-(methyl(2,2,6,6-tetramethylpiperidin-4-yl)amino)pyridazin-3-yl)-5-(2-methyl-1H-imidazol-4-yl)phenol; 5-(1,2-dimethyl-1H-imidazol-4-yl)-2-(6-(methyl(2,2,6,6-tetramethylpiperidin-4-yl)amino)pyridazin-3-yl)phenol; 1-(3-hy droxy-4-(6-(methyl(2,2,6,6-tetramethylpiperidin-4-yl)amino)pyridazin-3-yl)phenyl)-1H-pyrazole-4-carboxamide; 2-(6-((3aR,6aS)-5-(2-hydroxyethyl)hexahydropyrrolo[3,4-c]pyrrol-2(1H)-yl)pyridazin-3-yl)-5-(1H-pyrazol-4-yl)phenol; 2-(6-((3aR,6aS)-hexahydropyrrolo[3,4-c]pyrrol-2(1H)-yl)pyridazin-3-yl)-5-(1H-pyrazol-4-yl)phenol; 2-(6-((3aR,6a8)-5-methylhexahy dropyrrolo| 3,4-c|pyrrol-2(1H)-yl)pyridazin-3-yl)-5-(1H-pyrazol-4-yl)phenol; 4-(3-hydroxy-4-(6-(5-methylhexahydropyrrolo[3,4-c]pyrrol-2(1H)-yl)pyridazin-3-yl)phenyl)-1-methylpyridin-2(1H)-one; 4-(3-hydroxy-4-(6-((3aR,6aR)-1-methylhexahydropyrrolo[3,4-b]pyrrol-5(1H)-yl)pyridazin-3-yl)phenyl)-1-methylpyridin-2(1H)-one; 2-(6-(2,7-diazaspiro[4.5]decan-2-yl)pyridazin-3-yl)-5-(1H-pyrazol-4-yl)phenol; and 4-(4-(6-(2,7-diazaspiro[4.5]decan-2-yl)pyridazin-3-yl)-3-hydroxyphenyl)- 1-methylpyridin-2(1H)-one.

An additional representative splice modifier is RG7916 (Roche/PTC/SMAF,³⁵ 7-(4,7-diazaspiro[2.5]octan-7-yl)-2-(2,8-dimethylimidazo[1,2-b]pyridazin-6-yl)-4H-pyrido[1,2-a]pyrimidin-4-one) having the following structure:

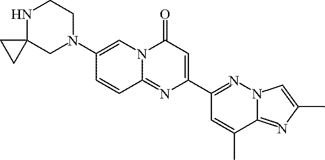 [SMILES: Cc1cn2nc(-c3cc(=O)n4cc(N5CCNC6(CC6)C5)ccc4n3)cc(C)c2n1]

An additional representative splice modifier is RG7800 (Roche) having the following structure:

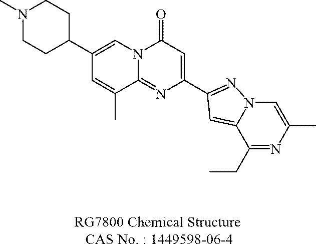

Analogues of splice modifiers such as RG7916 and RG7800 that can be used also are included, for example, 2-(2-methylimidazo[1,2-b]pyridazin-6-yl)-7-(4-methylpiperazin-1-yl)pyrido[1,2-a]pyrimidin-4-one; 7-[(8aR)-3,4,6,7,8,8a-hexahydro-1H-pyrrolo[1,2-a]pyrazin-2-yl]-2-(2-methylimidazo[1,2-b]pyridazin-6-yl)pyrido[1,2-a]pyrimidin-4-one; 7-[(8aS)-3,4,6,7,8,8a-hexahydro-1H-pyrrolo[1,2-a]pyrazin-2-yl]-2-(2,8-dimethylimidazo[1,2-b]pyridazin-6-yl)pyrido[1,2-a]pyrimidin-4-one; 7-[(8aR)-3,4,6,7,8,8a-hexahydro-1H-pyrrolo[1,2-a]pyrazin-2-yl]-2-(2,8-dimethylimidazo[1,2-b]pyridazin-6-yl)pyrido[1,2-a]pyrimidin-4-one; 7-[(8aS)-8a-methyl-1,3,4,6,7,8-hexahydropyrrolo[1,2-a]pyrazin-2-yl]-2-(2,8-dimethylimidazo[1,2-b]pyridazin-6-yl)pyrido[1,2-a]pyrimidin-4-one; 7-[(8aR)-8a-methyl-1,3,4,6,7,8-hexahydropyrrolo[1,2-a]pyrazin-2-yl]-2-(2,8-dimethylimidazo[1,2-b]pyridazin-6-yl)pyrido[1,2-a]pyrimidin-4-one; 2-(2,8-dimethylimidazo[1,2-b]pyridazin-6-yl)-7-[(3S,5R)-3,5-dimethylpiperazin-1-yl]pyrido[1,2-a]pyrimidin-4-one; 2-(2,8-dimethylimidazo[1,2-b]pyridazin-6-yl)-7-[(3S)-3-methylpiperazin-1-yl]pyrido[1,2-a]pyrimidin-4-one; 2-(2,8-dimethylimidazo[1,2-b]pyridazin-6-yl)-7-[(3R)-3-methylpiperazin-1-yl]pyrido[1,2-a]pyrimidin-4-one; 7-(1,4-diazepan-1-yl)-2-(2,8-dimethylimidazo[1,2-b]pyridazin-6-yl)pyrido[1,2-a]pyrimidin-4-one; 2-(2-methylimidazo[1,2-b]pyridazin-6-yl)-7-[(3S)-3-methylpiperazin-1-yl]pyrido[1,2-a]pyrimidin-4-one; 2-(2-methylimidazo[1,2-b]pyridazin-6-yl)-7-[(3R)-3-methylpiperazin-1-yl]pyrido[1,2-a]pyrimidin-4-one; 7-(1,4-diazepan-1-yl)-2-(2-methylimidazo[1,2-b]pyridazin-6-yl)pyrido[1,2-a]pyrimidin-4-one; 7-[(3R,5S)-3,5-dimethylpiperazin-1-yl]-2-(2-methylimidazo[1,2-b]pyridazin-6-yl)pyrido[1,2-a]pyrimidin-4-one; 7-[(8aS)-3,4,6,7,8,8a-hexahydro-1H-pyrrolo[1,2-a]pyrazin-2-yl]-2-(2-methylimidazo[1,2-b]pyridazin-6-yl)pyrido[1,2-a]pyrimidin-4-one; 7-[(8aS)-8a-methyl-1,3,4,6,7,8-hexahydropyrrolo[1,2-a]pyrazin-2-yl]-2-(2-methylimidazo[1,2-b]pyridazin-6-yl)pyrido[1,2-a]pyrimidin-4-one; 7-[(8aR)-8a-methyl-1,3,4,6,7,8-hexahydropyrrolo[1,2-a]pyrazin-2-yl]-2-(2-methylimidazo[1,2-b]pyridazin-6-yl)pyrido[1,2-a]pyrimidin-4-one; 2-(2,8-dimethylimidazo[1,2-b]pyridazin-6-yl)-7-[(3R)-3-pyrrolidin-1-ylpyrrolidin-1-yl]pyrido[1,2-a]pyrimidin-4-one; 7-(4,7-diazaspiro[2.5]octan-7-yl)-2-(2-methylimidazo[1,2-b]pyridazin-6-yl)pyrido[1,2-a]pyrimidin-4-one; 7-(4,7-diazaspiro[2.5]octan-7-yl)-2-(2,8-dimethylimidazo[1,2-b]pyridazin-6-yl)pyrido[1,2-a]pyrimidin-4-one; 2-(2-methylimidazo[1,2-b]pyridazin-6-yl)-7-[(3R)-3-pyrrolidin-1-ylpyrrolidin-1-yl]pyrido[1,2-a]pyrimidin-4-one; 2-(2,8-dimethylimidazo[1,2-b]pyridazin-6-yl)-7-(3,3-dimethylpiperazin-1-yl)pyrido[1,2-a]pyrimidin-4-one; 7-(3,3-dimethylpiperazin-1-yl)-2-(2-methylimidazo[1,2-b]pyridazin-6-yl)pyrido[1,2-a]pyrimidin-4-one; 2-(2,8-dimethylimidazo[1,2-b]pyridazin-6-yl)-9-methyl-7-[(3S)-3-methylpiperazin-1-yl]pyrido[1,2-a]pyrimidin-4-one; 2-(2,8-dimethylimidazo[1,2-b]pyridazin-6-yl)-9-methyl-7-[(3R)-3-methylpiperazin-1-yl]pyrido[1,2-a]pyrimidin-4-one; 2-(2,8-dimethylimidazo[1,2-b]pyridazin-6-yl)-7-[(3R,5S)-3,5-dimethylpiperazin-1-yl]-9-methyl-pyrido[1,2-a]pyrimidin-4-one; 2-(2,8-dimethylimidazo[1,2-b]pyridazin-6-yl)-7-(3,3-dimethylpiperazin-1-yl)-9-methyl-pyrido[1,2-a]pyrimidin-4-one; 7-(4,7-diazaspiro[2.5] octan-7-yl)-2-(2, 8-dimethylimidazo[1,2-b]pyridazin-6-yl)-9-methyl-pyrido[1,2-a]pyrimidin-4-one; 2-(2,8-dimethylimidazo[1,2-b]pyridazin-6-yl)-7-[(3S,5S)-3,5-dimethylpiperazin-1-yl]pyrido[1,2-a]pyrimidin-4-one; 2-(2,8-dimethylimidazo[1,2-b]pyridazin-6-yl)-7-[(3S)-3-pyrrolidin-1-ylpyrrolidin-1-yl]pyrido[1,2-a]pyrimidin-4-one; 2-(2-methylimidazo[1,2-b]pyridazin-6-yl)-7-[(3S)-3-pyrrolidin-1-ylpyrrolidin-1-yl]pyrido[1,2-a]pyrimidin-4-one; 7-[(3S,5S)-3,5-dimethylpiperazin-1-yl]-2-(2-methylimidazo[1,2-b]pyridazin-6-yl)pyrido[1,2-a]pyrimidin-4-one; 9-methyl-2-(2-methylimidazo[1,2-b]pyridazin-6-yl)-7-[(3S)-3-methylpiperazin-1-yl]pyrido[1,2-a]pyrimidin-4-one; 9-methyl-2-(2-methylimidazo[1,2-b]pyridazin-6-yl)-7-[(3R)-3-methylpiperazin-1-yl]pyrido[1,2-a]pyrimidin-4-one; 7-[(3R,5S)-3,5-dimethylpiperazin-1-yl]-9-methyl-2-(2-methylimidazo[1,2-b]pyridazin-6-yl)pyrido[1,2-a]pyrimidin-4-one; 7-(3,3-dimethylpiperazin-1-yl)-9-methyl-2-(2-methylimidazo[1,2-b]pyridazin-6-yl)pyrido[1,2-a]pyrimidin-4-one; 7-(4,7-diazaspiro[2.5]octan-7-yl)-9-methyl-2-(2-methylimidazo[1,2-b]pyridazin-6-y1)pyrido[1,2-a]pyrimidin-4-one; 7-[(3S,5S)-3,5-dimethylpiperazin-1-yl]-9-methyl-2-(2-methylimidazo[1,2-b]pyridazin-6-yl)pyrido[1,2-a]pyrimidin-4-one; and 7-[(3R)-3-ethylpiperazin-1-yl]-2-(2-methylimidazo[1,2-b]pyridazin-6-yl)pyrido[1,2-a]pyrimidin-4-one; 7-[(8aS)-3,4,6,7,8,8a-hexahydro-1H-pyrrolo[1,2-a]pyrazin-2-yl]-2-(2,8-dimethylimidazo[1,2-b]pyridazin-6-yl)pyrido[1,2-a]pyrimidin-4-one; 7-[(8aR)-3,4,6,7,8,8a-hexahydro-1H-pyrrolo[1,2-a]pyrazin-2-yl]-2-(2,8-dimethylimidazo[1,2-b]pyridazin-6-yl)pyrido[1,2-a]pyrimidin-4-one; 2-(2,8-dimethylimidazo[1,2-b]pyridazin-6-yl)-7-[(3S,5R)-3,5-dimethylpiperazin-1-yl]pyrido[1,2-a]pyrimidin-4-one; 7-[(3R,5S)-3,5-dimethylpiperazin-1-yl]-2-(2-methylimidazo[1,2-b]pyridazin-6-yl)pyrido[1,2-a]pyrimidin-4-one; 7-[(8aS)-3,4,6,7,8,8a-hexahydro-1H-pyrrolo[1,2-a]pyrazin-2-yl]-2-(2-methylimidazo[1,2-b]pyridazin-6-yl)pyrido[1,2-a]pyrimidin-4-one; 2-(2,8-dimethylimidazo[1,2-b]pyridazin-6-yl)-9-methyl-7-[(3S)-3-methylpiperazin-1-yl]pyrido[1,2-a]pyrimidin-4-one; 7-fluoro-2-(2-methylimidazo [1,2-b]pyridazin-6-yl)pyrido[1,2-a]pyrimidin-4-one; 2-(2,8-dimethylimidazo[1,2-b]pyridazin-6-yl)-7-fluoro-pyrido[1,2-a]pyrimidin-4-one; 7-fluoro-9-methyl-2-(2-methylimidazo[1,2-b]pyridazin-6-yl)pyrido[1,2-a]pyrimidin-4-one; 2-(2,8-dimethylimidazo[1,2-b]pyridazin-6-yl)-7-fluoro-9-methyl-pyrido[1,2-a]pyrimidin-4-one; or a pharmaceutically acceptable salt thereof.

### V. Methods of Administration

Any suitable cell or mammal can be administered or treated by a method or use described herein. Typically, a mammal is in need of a method described herein, that is suspected of having or expressing an abnormal or aberrant protein that is associated with a disease state.

Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In certain embodiments a mammal is a human. In certain embodiments a mammal is a non-rodent mammal (e.g., human, pig, goat, sheep, horse, dog, or the like). In certain embodiments a non-rodent mammal is a human. A mammal can be any age or at any stage of development (e.g., an adult, teen, child, infant, or a mammal in utero). A mammal can be male or female. In certain embodiments a mammal can be an animal disease model, for example, animal models having or expressing an abnormal or aberrant protein that is associated with a disease state or animal models with insufficient expression of a protein, which causes a disease state.

Mammals (subjects) treated by a method or composition described herein include adults (18 years or older) and children (less than 18 years of age). Adults include the elderly. Representative adults are 50 years or older. Children range in age from 1-2 years old, or from 2-4, 4-6, 6-18, 8-10, 10-12, 12-15 and 15-18 years old. Children also include infants. Infants typically range from 1-12 months of age.

In certain embodiments, a method includes administering a plurality of viral particles or nanoparticles to a mammal as set forth herein, where severity, frequency, progression or time of onset of one or more symptoms of a disease state, such as a neuro-degenerative disease, decreased, reduced, prevented, inhibited or delayed. In certain embodiments, a method includes administering a plurality of viral particles or nanoparticles to a mammal to treat an adverse symptom of a disease state, such as a neuro-degenerative disease. In certain embodiments, a method includes administering a plurality of viral particles or nanoparticles to a mammal to stabilize, delay or prevent worsening, or progression, or reverse and adverse symptom of a disease state, such as a neuro-degenerative disease.

In certain embodiments a method includes administering a plurality of viral particles or nanoparticles to the central nervous system, or portion thereof as set forth herein, of a mammal and severity, frequency, progression or time of onset of one or more symptoms of a disease state, such as a neuro-degenerative disease, are decreased, reduced, prevented, inhibited or delayed by at least about 5 to about 10, about 10 to about 25, about 25 to about 50, or about 50 to about 100 days.

In certain embodiments, a symptom or adverse effect comprises an early stage, middle or late stage symptom; a behavior, personality or language symptom; swallowing, movement, seizure, tremor or fidgeting symptom; ataxia; and/or a cognitive symptom such as memory, ability to organize.

In some embodiments, viral and non-viral based gene transfer methods can be used to introduce nucleic acids in mammalian cells or target tissues. Such methods can be used to administer nucleic acids encoding inhibitory RNAs, therapeutic proteins, or components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, 1992; Nabel & Feigner, 1993; Mitani & Caskey, 1993; Dillon, 1993; Miller, 1992; Van Brunt, 1988; Vigne, 1995; Kremer & Perricaudet, 1995; Haddada et al., 1995; and Yu et al., 1994.

Methods of non-viral delivery of nucleic acids include exosomes, lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in (e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91117424; WO 91116024. Delivery can be to cells (e.g. in vitro or ex vivo administration) or target tissues (e.g. in vivo administration).

In some embodiments, delivery is via the use of RNA or DNA viral based systems for the delivery of nucleic acids. Viral vectors in some aspects may be administered directly to patients (in vivo) or they can be used to treat cells in vitro or ex vivo, and then administered to patients. Viral-based systems in some embodiments include retroviral, lentivirus, adenoviral, adeno-associated and herpes simplex virus vectors for gene transfer.

### A. Viral Vectors

The term “vector” refers to small carrier nucleic acid molecule, a plasmid, virus (e.g., AAV vector, retroviral vector, lentiviral vector), or other vehicle that can be manipulated by insertion or incorporation of a nucleic acid. Vectors, such as viral vectors, can be used to introduce/transfer nucleic acid sequences into cells, such that the nucleic acid sequence therein is transcribed and, if encoding a protein, subsequently translated by the cells.

An “expression vector” is a specialized vector that contains a gene or nucleic acid sequence with the necessary regulatory regions needed for expression in a host cell. An expression vector may contain at least an origin of replication for propagation in a cell and optionally additional elements, such as a heterologous nucleic acid sequence, expression control element (e.g., a promoter, enhancer), intron, ITR(s), and polyadenylation signal.

A viral vector is derived from or based upon one or more nucleic acid elements that comprise a viral genome. Exemplary viral vectors include adeno-associated virus (AAV) vectors, retroviral vectors, and lentiviral vectors.

The term “recombinant,” as a modifier of vector, such as recombinant viral, e.g., lenti- or parvo-virus (e.g., AAV) vectors, as well as a modifier of sequences such as recombinant nucleic acid sequences and polypeptides, means that the compositions have been manipulated (i.e., engineered) in a fashion that generally does not occur in nature. A particular example of a recombinant vector, such as an AAV, retroviral, or lentiviral vector would be where a nucleic acid sequence that is not normally present in the wild-type viral genome is inserted within the viral genome. An example of a recombinant nucleic acid sequence would be where a nucleic acid (e.g., gene) encodes an inhibitory RNA cloned into a vector, with or without 5′, 3′ and/or intron regions that the gene is normally associated within the viral genome. Although the term “recombinant” is not always used herein in reference to vectors, such as viral vectors, as well as sequences such as polynucleotides, “recombinant” forms including nucleic acid sequences, polynucleotides, transgenes, etc. are expressly included in spite of any such omission.

A recombinant viral “vector” is derived from the wild type genome of a virus, such as AAV, retrovirus, or lentivirus, by using molecular methods to remove the wild type genome from the virus, and replacing with a non-native nucleic acid, such as a nucleic acid sequence. Typically, for example, for AAV, one or both inverted terminal repeat (ITR) sequences of the AAV genome are retained in the recombinant AAV vector. A “recombinant” viral vector (e.g., rAAV) is distinguished from a viral (e.g., AAV) genome, since all or a part of the viral genome has been replaced with a non-native sequence with respect to the viral genomic nucleic acid such a nucleic acid encoding a transactivator or nucleic acid encoding an inhibitory RNA or nucleic acid encoding a therapeutic protein. Incorporation of such non-native nucleic acid sequences therefore defines the viral vector as a “recombinant” vector, which in the case of AAV can be referred to as a “rAAV vector.”

### 1. Adeno-Associated Virus

Adeno-associated virus (AAV) is a small nonpathogenic virus of the parvoviridae family. To date, numerous serologically distinct AAVs have been identified, and more than a dozen have been isolated from humans or primates. AAV is distinct from other members of this family by its dependence upon a helper virus for replication.

AAV genomes can exist in an extrachromosomal state without integrating into host cellular genomes; possess a broad host range; transduce both dividing and non-dividing cells in vitro and in vivo and maintain high levels of expression of the transduced genes. AAV viral particles are heat stable; resistant to solvents, detergents, changes in pH, and temperature; and can be column purified and/or concentrated on CsCl gradients or by other means. The AAV genome comprises a single-stranded deoxyribonucleic acid (ssDNA), either positive- or negative-sensed. The approximately 5 kb genome of AAV consists of one segment of single stranded DNA of either plus or minus polarity. The ends of the genome are short inverted terminal repeats (ITRs) that can fold into hairpin structures and serve as the origin of viral DNA replication.

An AAV “genome” refers to a recombinant nucleic acid sequence that is ultimately packaged or encapsulated to form an AAV particle. An AAV particle often comprises an AAV genome packaged with AAV capsid proteins. In cases where recombinant plasmids are used to construct or manufacture recombinant vectors, the AAV vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for propagation and recombinant virus production, but is not itself packaged or encapsulated into viral particles. Thus, an AAV vector “genome” refers to nucleic acid that is packaged or encapsulated by AAV capsid proteins.

The AAV virion (particle) is a non-enveloped, icosahedral particle approximately 25 nm in diameter. The AAV particle comprises an icosahedral symmetry comprised of three related capsid proteins, VP1, VP2 and VP3, which interact together to form the capsid. The right ORF often encodes the capsid proteins VP1, VP2, and VP3. These proteins are often found in a ratio of 1:1:10 respectively, but may be in varied ratios, and are all derived from the right-hand ORF. The VP1, VP2 and VP3 capsid proteins differ from each other by the use of alternative splicing and an unusual start codon. Deletion analysis has shown that removal or alteration of VP1 which is translated from an alternatively spliced message results in a reduced yield of infectious particles. Mutations within the VP3 coding region result in the failure to produce any single-stranded progeny DNA or infectious particles.

An AAV particle is a viral particle comprising an AAV capsid. In certain embodiments, the genome of an AAV particle encodes one, two or all VP1, VP2 and VP3 polypeptides.

The genome of most native AAVs often contain two open reading frames (ORFs), sometimes referred to as a left ORF and a right ORF. The left ORF often encodes the non-structural Rep proteins, Rep 40, Rep 52, Rep 68 and Rep 78, which are involved in regulation of replication and transcription in addition to the production of single-stranded progeny genomes. Two of the Rep proteins have been associated with the preferential integration of AAV genomes into a region of the q arm of human chromosome 19. Rep68/78 have been shown to possess NTP binding activity as well as DNA and RNA helicase activities. Some Rep proteins possess a nuclear localization signal as well as several potential phosphorylation sites. In certain embodiments the genome of an AAV (e.g., an rAAV) encodes some or all of the Rep proteins. In certain embodiments the genome of an AAV (e.g., an rAAV) does not encode the Rep proteins. In certain embodiments one or more of the Rep proteins can be delivered in trans and are therefore not included in an AAV particle comprising a nucleic acid encoding a polypeptide.

The ends of the AAV genome comprise short inverted terminal repeats (ITR) which have the potential to fold into T-shaped hairpin structures that serve as the origin of viral DNA replication. Accordingly, the genome of an AAV comprises one or more (e.g., a pair of) ITR sequences that flank a single stranded viral DNA genome. The ITR sequences often have a length of about 145 bases each. Within the ITR region, two elements have been described which are believed to be central to the function of the ITR, a GAGC repeat motif and the terminal resolution site (trs). The repeat motif has been shown to bind Rep when the ITR is in either a linear or hairpin conformation. This binding is thought to position Rep68/78 for cleavage at the trs which occurs in a site- and strand-specific manner. In addition to their role in replication, these two elements appear to be central to viral integration. Contained within the chromosome 19 integration locus is a Rep binding site with an adjacent trs. These elements have been shown to be functional and necessary for locus specific integration.

In certain embodiments, an AAV (e.g., a rAAV) comprises two ITRs. In certain embodiments, an AAV (e.g., a rAAV) comprises a pair of ITRs. In certain embodiments, an AAV (e.g., a rAAV) comprises a pair of ITRs that flank (i.e., are at each 5′ and 3′ end) of a nucleic acid sequence that at least encodes a polypeptide having function or activity.

An AAV vector (e.g., rAAV vector) can be packaged and is referred to herein as an “AAV particle” for subsequent infection (transduction) of a cell, ex vivo, in vitro or in vivo. Where a recombinant AAV vector is encapsulated or packaged into an AAV particle, the particle can also be referred to as a “rAAV particle.” In certain embodiments, an AAV particle is a rAAV particle. A rAAV particle often comprises a rAAV vector, or a portion thereof. A rAAV particle can be one or more rAAV particles (e.g., a plurality of AAV particles). rAAV particles typically comprise proteins that encapsulate or package the rAAV vector genome (e.g., capsid proteins). It is noted that reference to a rAAV vector can also be used to reference a rAAV particle.

Any suitable AAV particle (e.g., rAAV particle) can be used for a method or use herein. A rAAV particle, and/or genome comprised therein, can be derived from any suitable serotype or strain of AAV. A rAAV particle, and/or genome comprised therein, can be derived from two or more serotypes or strains of AAV. Accordingly, a rAAV can comprise proteins and/or nucleic acids, or portions thereof, of any serotype or strain of AAV, wherein the AAV particle is suitable for infection and/or transduction of a mammalian cell. Non-limiting examples of AAV serotypes include AAV1, AAV2, AAV3, AAV4, AAV5, AAV6, AAV7, AAV8, AAV9, AAV10, AAV11, AAV12, AAV-rh74, AAV-rh10 and AAV-2i8.

In certain embodiments a plurality of rAAV particles comprises particles of, or derived from, the same strain or serotype (or subgroup or variant). In certain embodiments a plurality of rAAV particles comprise a mixture of two or more different rAAV particles (e.g., of different serotypes and/or strains).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

In certain embodiments, a rAAV particle excludes certain serotypes. In one embodiment, a rAAV particle is not an AAV4 particle. In certain embodiments, a rAAV particle is antigenically or immunologically distinct from AAV4. Distinctness can be determined by standard methods. For example, ELISA and Western blots can be used to determine whether a viral particle is antigenically or immunologically distinct from AAV4. Furthermore, in certain embodiments a rAAV2 particle retains tissue tropism distinct from AAV4.

In certain embodiments, a rAAV vector based upon a first serotype genome corresponds to the serotype of one or more of the capsid proteins that package the vector. For example, the serotype of one or more AAV nucleic acids (e.g., ITRs) that comprises the AAV vector genome corresponds to the serotype of a capsid that comprises the rAAV particle.

In certain embodiments, a rAAV vector genome can be based upon an AAV (e.g., AAV2) serotype genome distinct from the serotype of one or more of the AAV capsid proteins that package the vector. For example, a rAAV vector genome can comprise AAV2 derived nucleic acids (e.g., ITRs), whereas at least one or more of the three capsid proteins are derived from a different serotype, e.g., an AAV1, AAV3, AAV4, AAV5, AAV6, AAV7, AAV8, AAV9, AAV10, AAV11, AAV12, Rh10, Rh74 or AAV-2i8 serotype or variant thereof.

In certain embodiments, a rAAV particle or a vector genome thereof related to a reference serotype has a polynucleotide, polypeptide or subsequence thereof that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a polynucleotide, polypeptide or subsequence of an AAV1, AAV2, AAV3, AAV4, AAV5, AAV6, AAV7, AAV8, AAV9, AAV10, AAV11, AAV12, Rh10, Rh74 or AAV-2i8 particle. In particular embodiments, a rAAV particle or a vector genome thereof related to a reference serotype has a capsid or ITR sequence that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a capsid or ITR sequence of an AAV1, AAV2, AAV3, AAV4, AAV5, AAV6, AAV7, AAV8, AAV9, AAV10, AAV11, AAV12, Rh10, Rh74 or AAV-2i8 serotype.

In certain embodiments, a method herein comprises use, administration or delivery of a rAAV1, rAAV2, rAAV3, rAAV4, rAAV5, rAAV6, rAAV7, rAAV8, rAAV9, rAAV10, rAAV11, rAAV12, rRh10, rRh74 or rAAV-2i8 particle.

In certain embodiments, a method herein comprises use, administration or delivery of a rAAV2 particle. In certain embodiments a rAAV2 particle comprises an AAV2 capsid. In certain embodiments a rAAV2 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments a rAAV2 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments, a rAAV2 particle is a variant of a native or wild-type AAV2 particle. In some aspects, one or more capsid proteins of an AAV2 variant have 1, 2, 3, 4, 5, 5-10, 10-15, 15-20 or more amino acid substitutions compared to capsid protein(s) of a native or wild-type AAV2 particle.

In certain embodiments a rAAV9 particle comprises an AAV9 capsid. In certain embodiments a rAAV9 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments a rAAV9 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments, a rAAV9 particle is a variant of a native or wild-type AAV9 particle. In some aspects, one or more capsid proteins of an AAV9 variant have 1, 2, 3, 4, 5, 5-10, 10-15, 15-20 or more amino acid substitutions compared to capsid protein(s) of a native or wild-type AAV9 particle.

In certain embodiments, a rAAV particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV1, AAV2, AAV3, AAV4, AAV5, AAV6, AAV7, AAV8, AAV9, AAV10, AAV11, AAV12, AAV-rh74, AAV-rh10 or AAV-2i8, as long as they retain one or more desired ITR functions (e.g., ability to form a hairpin, which allows DNA replication; integration of the AAV DNA into a host cell genome; and/or packaging, if desired).

In certain embodiments, a rAAV2 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication; integration of the AAV DNA into a host cell genome; and/or packaging, if desired).

In certain embodiments, a rAAV9 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication; integration of the AAV DNA into a host cell genome; and/or packaging, if desired).

A rAAV particle can comprise an ITR having any suitable number of “GAGC” repeats. In certain embodiments an ITR of an AAV2 particle comprises 1, 2, 3, 4, 5, 6, 7, 8, 9 or 10 or more “GAGC” repeats. In certain embodiments a rAAV2 particle comprises an ITR comprising three “GAGC” repeats. In certain embodiments a rAAV2 particle comprises an ITR which has less than four “GAGC” repeats. In certain embodiments a rAAV2 particle comprises an ITR which has more than four “GAGC” repeats. In certain embodiments an ITR of a rAAV2 particle comprises a Rep binding site wherein the fourth nucleotide in the first two “GAGC” repeats is a C rather than a T.

Exemplary suitable length of DNA can be incorporated in rAAV vectors for packaging/encapsidation into a rAAV particle can about 5 kilobases (kb) or less. In particular, embodiments, length of DNA is less than about 5kb, less than about 4.5 kb, less than about 4 kb, less than about 3.5 kb, less than about 3 kb, or less than about 2.5 kb.

rAAV vectors that include a nucleic acid sequence that directs the expression of an RNAi or polypeptide can be generated using suitable recombinant techniques known in the art (e.g., see Sambrook et al., 1989). Recombinant AAV vectors are typically packaged into transduction-competent AAV particles and propagated using an AAV viral packaging system. A transduction-competent AAV particle is capable of binding to and entering a mammalian cell and subsequently delivering a nucleic acid cargo (e.g., a heterologous gene) to the nucleus of the cell. Thus, an intact rAAV particle that is transduction-competent is configured to transduce a mammalian cell. A rAAV particle configured to transduce a mammalian cell is often not replication competent, and requires additional protein machinery to self-replicate. Thus, a rAAV particle that is configured to transduce a mammalian cell is engineered to bind and enter a mammalian cell and deliver a nucleic acid to the cell, wherein the nucleic acid for delivery is often positioned between a pair of AAV ITRs in the rAAV genome.

Suitable host cells for producing transduction-competent AAV particles include but are not limited to microorganisms, yeast cells, insect cells, and mammalian cells that can be, or have been, used as recipients of a heterologous rAAV vectors. Cells from the stable human cell line, HEK293 (readily available through, e.g., the American Type Culture Collection under Accession Number ATCC CRL1573) can be used. In certain embodiments a modified human embryonic kidney cell line (e.g., HEK293), which is transformed with adenovirus type-5 DNA fragments, and expresses the adenoviral E1a and E1b genes is used to generate recombinant AAV particles. The modified HEK293 cell line is readily transfected, and provides a particularly convenient platform in which to produce rAAV particles. Methods of generating high titer AAV particles capable of transducing mammalian cells are known in the art. For example, AAV particle can be made as set forth in Wright, 2008 and Wright, 2009.

In certain embodiments, AAV helper functions are introduced into the host cell by transfecting the host cell with an AAV helper construct either prior to, or concurrently with, the transfection of an AAV expression vector. AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products.

### 2. Retrovirus

Viral vectors for use as a delivered agent in the methods, compositions and uses herein include a retroviral vector (see e.g., Miller (1992) Nature, 357:455-460). Retroviral vectors are well suited for delivering nucleic acid into cells because of their ability to deliver an unrearranged, single copy gene into a broad range of rodent, primate and human somatic cells. Retroviral vectors integrate into the genome of host cells. Unlike other viral vectors, they only infect dividing cells.

Retroviruses are RNA viruses such that the viral genome is RNA. When a host cell is infected with a retrovirus, the genomic RNA is reverse transcribed into a DNA intermediate, which is integrated very efficiently into the chromosomal DNA of infected cells. This integrated DNA intermediate is referred to as a provirus. Transcription of the provirus and assembly into infectious virus occurs in the presence of an appropriate helper virus or in a cell line containing appropriate sequences permitting encapsulation without coincident production of a contaminating helper virus. A helper virus is not required for the production of the recombinant retrovirus if the sequences for encapsulation are provided by co-transfection with appropriate vectors.

The retroviral genome and the proviral DNA have three genes: the gag, the pol and the env, which are flanked by two long terminal repeat (LTR) sequences. The gag gene encodes the internal structural (matrix, capsid, and nucleocapsid) proteins and the env gene encodes viral envelope glycoproteins. The pol gene encodes products that include the RNA-directed DNA polymerase reverse transcriptase that transcribes the viral RNA into double-stranded DNA, integrase that integrate the DNA produced by reverse transcriptase into host chromosomal DNA, and protease that acts to process the encoded gag and pol genes. The 5′ and 3′ LTRs serve to promote transcription and polyadenylation of the virion RNAs. The LTR contains all other cis-acting sequences necessary for viral replication.

Retroviral vectors are described by Coffin et al., Retroviruses, Cold Spring Harbor Laboratory Press (1997). Exemplary of a retrovirus is Moloney murine leukemia virus (MMLV) or the murine stem cell virus (MSCV). Retroviral vectors can be replication-competent or replication-defective. Typically, a retroviral vector is replication-defective in which the coding regions for genes necessary for additional rounds of virion replication and packaging are deleted or replaced with other genes. Consequently, the viruses are not able to continue their typical lytic pathway once an initial target cell is infected. Such retroviral vectors, and the necessary agents to produce such viruses (e.g., packaging cell line) are commercially available (see, e.g., retroviral vectors and systems available from Clontech, such as Catalog number 634401, 631503, 631501, and others, Clontech, Mountain View, Calif.).

Such retroviral vectors can be produced as delivered agents by replacing the viral genes required for replication with the nucleic acid molecule to be delivered. The resulting genome contains an LTR at each end with the desired gene or genes in between. Methods of producing retrovirus are known to one of skill in the art (see, e.g., International published PCT Application No. WO1995/026411). The retroviral vector can be produced in a packaging cell line containing a helper plasmid or plasmids. The packaging cell line provides the viral proteins required for capsid production and the virion maturation of the vector (e.g., gag, pol and env genes). Typically, at least two separate helper plasmids (separately containing the gag and pol genes; and the env gene) are used so that recombination between the vector plasmid cannot occur. For example, the retroviral vector can be transferred into a packaging cell line using standard methods of transfection, such as calcium phosphate mediated transfection. Packaging cell lines are well known to one of skill in the art, and are commercially available. An exemplary packaging cell line is GP2-293 packaging cell line (Catalog Numbers 631505, 631507, 631512, Clontech). After sufficient time for virion product, the virus is harvested. If desired, the harvested virus can be used to infect a second packaging cell line, for example, to produce a virus with varied host tropism. The end result is a replicative incompetent recombinant retrovirus that includes the nucleic acid of interest but lacks the other structural genes such that a new virus cannot be formed in the host cell.

References illustrating the use of retroviral vectors in gene therapy include: Clowes et al., (1994) J. Clin. Invest. 93:644-651; Kiem et al., (1994) Blood 83:1467-1473; Salmons and Gunzberg (1993) Human Gene Therapy 4:129-141; Grossman and Wilson (1993) Curr. Opin. in Genetics and Devel. 3:110-114; Sheridan (2011) Nature Biotechnology, 29:121; Cassani et al. (2009) Blood, 114:3546-3556.

### 3. Lentivirus

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe. Lentiviral vectors are well known in the art (see, e.g., U.S. Patents 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell, wherein a suitable host cell is transfected with two or more vectors carrying the packaging functions, namely gag, pol and env, as well as rev and tat, is described in U.S. Pat. 5,994,136, incorporated herein by reference.

The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTRs serve to promote transcription and polyadenylation of the virion RNAs. The LTR contains all other cis-acting sequences necessary for viral replication. Lentiviruses have additional genes including vif, vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

### 4. Other Viral Vectors

The development and utility of viral vectors for gene delivery is constantly improving and evolving. Other viral vectors such as poxvirus; e.g., vaccinia virus (Gnant et al., 1999; Gnant et al., 1999), alpha virus; e.g., sindbis virus, Semliki forest virus (Lundstrom, 1999), reovirus (Coffey et al., 1998) and influenza A virus (Neumann et al., 1999) are contemplated for use in the present disclosure and may be selected according to the requisite properties of the target system.

### 5. Chimeric Viral Vectors

Chimeric or hybrid viral vectors are being developed for use in therapeutic gene delivery and are contemplated for use in the present disclosure. Chimeric poxviral/retroviral vectors (Holzer et al., 1999), adenoviral/retroviral vectors (Feng et al., 1997; Bilbao et al., 1997; Caplen et al., 2000) and adenoviral/adeno-associated viral vectors (Fisher et al., 1996; U.S. Pat. 5,871,982) have been described. These “chimeric” viral gene transfer systems can exploit the favorable features of two or more parent viral species. For example, Wilson et al., provide a chimeric vector construct which comprises a portion of an adenovirus, AAV 5′ and 3′ ITR sequences and a selected transgene, described below (U.S. Pat. 5,871,983, specifically incorporate herein by reference).

### B. Nanoparticles

### 1. Lipid-based Nanoparticles

In some embodiments, a lipid-based nanoparticle is a liposome, an exosome, a lipid preparation, or another lipid-based nanoparticle, such as a lipid-based vesicle (e.g., a DOTAP:cholesterol vesicle). Lipid-based nanoparticles may be positively charged, negatively charged, or neutral.

### A. Liposomes

A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition. Liposomes provided herein include unilamellar liposomes, multilamellar liposomes, and multivesicular liposomes. Liposomes provided herein may be positively charged, negatively charged, or neutrally charged. In certain embodiments, the liposomes are neutral in charge.

A multilamellar liposome has multiple lipid layers separated by aqueous medium. Such liposomes form spontaneously when lipids comprising phospholipids are suspended in an excess of aqueous solution. The lipid components undergo self-rearrangement before the formation of closed structures and entrap water and dissolved solutes between the lipid bilayers. Lipophilic molecules or molecules with lipophilic regions may also dissolve in or associate with the lipid bilayer.

In specific aspects, a polypeptide, a nucleic acid, or a small molecule drug may be, for example, encapsulated in the aqueous interior of a liposome, interspersed within the lipid bilayer of a liposome, attached to a liposome via a linking molecule that is associated with both the liposome and the polypeptide/nucleic acid, entrapped in a liposome, complexed with a liposome, or the like.

A liposome used according to the present embodiments can be made by different methods, as would be known to one of ordinary skill in the art. For example, a phospholipid, such as for example the neutral phospholipid dioleoylphosphatidylcholine (DOPC), is dissolved in tert-butanol. The lipid(s) is then mixed with a polypeptide, nucleic acid, and/or other component(s). Tween 20 is added to the lipid mixture such that Tween 20 is about 5% of the composition’s weight. Excess tert-butanol is added to this mixture such that the volume of tert-butanol is at least 95%. The mixture is vortexed, frozen in a dry ice/acetone bath and lyophilized overnight. The lyophilized preparation is stored at -20° C. and can be used up to three months. When required the lyophilized liposomes are reconstituted in 0.9% saline.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min to 2 h, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

The dried lipids or lyophilized liposomes prepared as described above may be dehydrated and reconstituted in a solution of a protein or peptide and diluted to an appropriate concentration with a suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000 × g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

Additional liposomes which may be useful with the present embodiments include cationic liposomes, for example, as described in WO02/100435A1, U.S Pat. 5,962,016, U.S. Application 2004/0208921, WO03/015757A1, WO04029213A2, U.S. Pat. 5,030,453, and U.S. Pat. 6,680,068, all of which are hereby incorporated by reference in their entirety without disclaimer.

In preparing such liposomes, any protocol described herein, or as would be known to one of ordinary skill in the art may be used. Additional non-limiting examples of preparing liposomes are described in U.S. Patents 4,728,578, 4,728,575, 4,737,323, 4,533,254, 4,162,282, 4,310,505, and 4,921,706; International Applications PCT/US85/01161 and PCT/US89/05040, each incorporated herein by reference.

In certain embodiments, the lipid-based nanoparticle is a neutral liposome (e.g., a DOPC liposome). “Neutral liposomes” or “non-charged liposomes”, as used herein, are defined as liposomes having one or more lipid components that yield an essentially-neutral, net charge (substantially non-charged). By “essentially neutral” or “essentially non-charged”, it is meant that few, if any, lipid components within a given population (e.g., a population of liposomes) include a charge that is not canceled by an opposite charge of another component (i.e., fewer than 10% of components include a non-canceled charge, more preferably fewer than 5%, and most preferably fewer than 1%). In certain embodiments, neutral liposomes may include mostly lipids and/or phospholipids that are themselves neutral under physiological conditions (i.e., at about pH 7).

Liposomes and/or lipid-based nanoparticles of the present embodiments may comprise a phospholipid. In certain embodiments, a single kind of phospholipid may be used in the creation of liposomes (e.g., a neutral phospholipid, such as DOPC, may be used to generate neutral liposomes). In other embodiments, more than one kind of phospholipid may be used to create liposomes. Phospholipids may be from natural or synthetic sources. Phospholipids include, for example, phosphatidylcholines, phosphatidylglycerols, and phosphatidylethanolamines; because phosphatidylethanolamines and phosphatidyl cholines are non-charged under physiological conditions (i.e., at about pH 7), these compounds may be particularly useful for generating neutral liposomes. In certain embodiments, the phospholipid DOPC is used to produce non-charged liposomes. In certain embodiments, a lipid that is not a phospholipid (e.g., a cholesterol) may be used

Phospholipids include glycerophospholipids and certain sphingolipids. Phospholipids include, but are not limited to, dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), distearoyl sphingomyelin (“DSSP”), distearoylphophatidylethanolamine (“DSPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, and dilinoleoylphosphatidylcholine.

### B. Exosomes

“Extracellular vesicles” and “EVs” are cell-derived and cell-secreted microvesicles which, as a class, include exosomes, exosome-like vesicles, ectosomes (which result from budding of vesicles directly from the plasma membrane), microparticles, microvesicles, shedding microvesicles (SMVs), nanoparticles and even (large) apoptotic blebs or bodies (resulting from cell death) or membrane particles.

The terms “microvesicle” and “exosomes,” as used herein, refer to a membranous particle having a diameter (or largest dimension where the particles is not spheroid) of between about 10 nm to about 5000 nm, more typically between 30 nm and 1000 nm, and most typically between about 50 nm and 750 nm, wherein at least part of the membrane of the exosomes is directly obtained from a cell. Most commonly, exosomes will have a size (average diameter) that is up to 5% of the size of the donor cell. Therefore, especially contemplated exosomes include those that are shed from a cell.

Exosomes may be detected in or isolated from any suitable sample type, such as, for example, body fluids. As used herein, the term “isolated” refers to separation out of its natural environment and is meant to include at least partial purification and may include substantial purification. As used herein, the term “sample” refers to any sample suitable for the methods provided by the present invention. The sample may be any sample that includes exosomes suitable for detection or isolation. Sources of samples include blood, bone marrow, pleural fluid, peritoneal fluid, cerebrospinal fluid, urine, saliva, amniotic fluid, malignant ascites, broncho-alveolar lavage fluid, synovial fluid, breast milk, sweat, tears, joint fluid, and bronchial washes. In one aspect, the sample is a blood sample, including, for example, whole blood or any fraction or component thereof. A blood sample suitable for use with the present invention may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with cancer.

Exosomes may also be isolated from tissue samples, such as surgical samples, biopsy samples, tissues, feces, and cultured cells. When isolating exosomes from tissue sources it may be necessary to homogenize the tissue in order to obtain a single cell suspension followed by lysis of the cells to release the exosomes. When isolating exosomes from tissue samples it is important to select homogenization and lysis procedures that do not result in disruption of the exosomes. Exosomes contemplated herein are preferably isolated from body fluid in a physiologically acceptable solution, for example, buffered saline, growth medium, various aqueous medium, etc.

Exosomes may be isolated from freshly collected samples or from samples that have been stored frozen or refrigerated. In some embodiments, exosomes may be isolated from cell culture medium. Although not necessary, higher purity exosomes may be obtained if fluid samples are clarified before precipitation with a volume-excluding polymer, to remove any debris from the sample. Methods of clarification include centrifugation, ultracentrifugation, filtration, or ultrafiltration. Most typically, exosomes can be isolated by numerous methods well-known in the art. One preferred method is differential centrifugation from body fluids or cell culture supernatants. Exemplary methods for isolation of exosomes are described in (Losche et al., 2004; Mesri and Altieri, 1998; Morel et al., 2004). Alternatively, exosomes may also be isolated via flow cytometry as described in (Combes et al., 1997).

One accepted protocol for isolation of exosomes includes ultracentrifugation, often in combination with sucrose density gradients or sucrose cushions to float the relatively low-density exosomes. Isolation of exosomes by sequential differential centrifugations is complicated by the possibility of overlapping size distributions with other microvesicles or macromolecular complexes. Furthermore, centrifugation may provide insufficient means to separate vesicles based on their sizes. However, sequential centrifugations, when combined with sucrose gradient ultracentrifugation, can provide high enrichment of exosomes.

Isolation of exosomes based on size, using alternatives to the ultracentrifugation routes, is another option. Successful purification of exosomes using ultrafiltration procedures that are less time consuming than ultracentrifugation, and do not require use of special equipment have been reported. Similarly, a commercial kit is available (EXOMIR™, Bioo Scientific) which allows removal of cells, platelets, and cellular debris on one microfilter and capturing of vesicles bigger than 30 nm on a second microfilter using positive pressure to drive the fluid. However, for this process, the exosomes are not recovered, their RNA content is directly extracted from the material caught on the second microfilter, which can then be used for PCR analysis. HPLC-based protocols could potentially allow one to obtain highly pure exosomes, though these processes require dedicated equipment and are difficult to scale up. A significant problem is that both blood and cell culture media contain large numbers of nanoparticles (some non-vesicular) in the same size range as exosomes. For example, some miRNAs may be contained within extracellular protein complexes rather than exosomes; however, treatment with protease (e.g., proteinase K) can be performed to eliminate any possible contamination with “extraexosomal” protein.

In another embodiment, exosomes may be captured by techniques commonly used to enrich a sample for exosomes, such as those involving immunospecific interactions (e.g., immunomagnetic capture). Immunomagnetic capture, also known as immunomagnetic cell separation, typically involves attaching antibodies directed to proteins found on a particular cell type to small paramagnetic beads. When the antibody-coated beads are mixed with a sample, such as blood, they attach to and surround the particular cell. The sample is then placed in a strong magnetic field, causing the beads to pellet to one side. After removing the blood, captured cells are retained with the beads. Many variations of this general method are well-known in the art and suitable for use to isolate exosomes. In one example, the exosomes may be attached to magnetic beads (e.g., aldehyde/sulphate beads) and then an antibody is added to the mixture to recognize an epitope on the surface of the exosomes that are attached to the beads.

As will be appreciated by one of skill in the art, prior or subsequent to loading with cargo, exosomes may be further altered by inclusion of a targeting moiety to enhance the utility thereof as a vehicle for delivery of cargo. In this regard, exosomes may be engineered to incorporate an entity that specifically targets a particular cell to tissue type. This target-specific entity, e.g., peptide having affinity for a receptor or ligand on the target cell or tissue, may be integrated within the exosomal membrane, for example, by fusion to an exosomal membrane marker using methods well-established in the art.

### 2. Nonlipid Nanoparticles

Spherical Nucleic Acid (SNA™) constructs and other nanoparticles (particularly gold nanoparticles) are also contemplated as a means to deliver chimeric minigenes to intended target cells. Due to their dense loading, a majority of cargo (e.g., DNA) remains bound to the constructs inside cells, conferring nucleic acid stability and resistance to enzymatic degradation. For all cell types studied (e.g., neurons, tumor cell lines, etc.) the constructs demonstrate a transfection efficiency of 99% with no need for carriers or transfection agents. The unique target binding affinity and specificity of the constructs allow exquisite specificity for matched target sequences (i.e., limited off-target effects). The constructs significantly outperform leading conventional transfection reagents (Lipofectamine 2000 and Cytofectin). The constructs can enter a variety of cultured cells, primary cells, and tissues with no apparent toxicity. The constructs elicit minimal changes in global gene expression as measured by whole-genome microarray studies and cytokine-specific protein assays. Any number of single or combinatorial agents (e.g., proteins, peptides, small molecules) can be used to tailor the surface of the constructs. See, e.g., Jensen et al., Sci. Transl. Med. 5, 209ra152 (2013).

Self-assembling nanoparticles with nucleic acid cargo may be constructed with polyethyleneimine (PEI) that is PEGylated with an Arg-Gly-Asp (RGD) peptide ligand attached at the distal end of the polyethylene glycol (PEG). Nanoplexes may be prepared by mixing equal volumes of aqueous solutions of cationic polymer and nucleic acid to give a net molar excess of ionizable nitrogen (polymer) to phosphate (nucleic acid) over the range of 2 to 6. The electrostatic interactions between cationic polymers and nucleic acid resulted in the formation of polyplexes with average particle size distribution of about 100 nm, hence referred to here as nanoplexes (see, e.g., Bartlett et al., PNAS, 104:39, 2007).

### C. Encapsulated Cell Implantation

The chimeric minigenes herein can be delivered ex vivo to cells, which are then encapsulated and implanted in order to deliver the target gene to a patient. For example, cells isolated from a patient or a donor introduced with an exogenous heterologous nucleic acid can be delivered directly to a patient by implantation of encapsulated cells. The advantage of implantation of encapsulated cells is that the immune response to the cells is reduced by the encapsulation. Thus, provided herein is a method of administering a genetically modified cell or cells to a subject. The number of cells that are delivered depends on the desired effect, the particular nucleic acid, the subject being treated and other similar factors, and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, or fetal liver. For example, the genetically modified cells can be pluripotent or totipotent stem cells (including induced pluripotent stem cells) or can be embryonic, fetal, or fully differentiated cells. The genetically modified cells can be cells from the same subject or can be cells from the same or different species as the recipient subject. In a preferred example, the cell used for gene therapy is autologous to the patient. Methods of genetically modifying cells and transplanting cells are known in the art.

Typically, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see e.g., Loeffler and Behr, Meth. Enzymol. (1993) 217:599-618; Cotten et al., Meth. Enzymol. (1993) 217:618-644; Cline, Pharmac. Ther. (1985) 29:69-92) and can be used provided that the necessary developmental and physiological functions of the recipient cells are not disrupted. In particular examples, the method is one that permits stable transfer of the nucleic acid to the cell, so that the nucleic acid is expressible by the cell and heritable and expressible by its cell progeny.

Encapsulation can be performed using an alginate microcapsule coated with an alginate/polylysine complex. Hydrogel microcapsules have been extensively investigated for encapsulation of living cells or cell aggregates for tissue engineering and regenerative medicine (Orive, et al. Nat. Medicine 2003, 9, 104; Paul, et al., Regen. Med. 2009, 4, 733; Read, et al. Biotechnol. 2001, 19, 29) In general, capsules are designed to allow facile diffusion of oxygen and nutrients to the encapsulated cells, while releasing the therapeutic proteins secreted by the cells, and to protect the cells from attack by the immune system. These have been developed as potential therapeutics for a range of diseases including type I diabetes, cancer, and neurodegenerative disorders such as Parkinson’s (Wilson et al. Adv. Drug. Deliv. Rev. 2008, 60, 124; Joki, et al. Nat. Biotech. 2001, 19, 35; Kishima, et al. Neurobiol. Dis. 2004, 16, 428). One of the most common capsule formulations is based on alginate hydrogels, which can be formed through ionic crosslinking. In a typical process, the cells are first blended with a viscous alginate solution. The cell suspension is then processed into micro-droplets using different methods such as air shear, acoustic vibration or electrostatic droplet formation (Rabanel et al. Biotechnol. Prog. 2009, 25, 946). The alginate droplet is gelled upon contact with a solution of divalent ions, such as Ca2+ or Ba2+.

Capsules are disclosed for transplanting mammalian cells into a subject. The capsules are formed from a biocompatible, hydrogel-forming polymer encapsulating the cells to be transplanted. In order to inhibit capsular overgrowth (fibrosis), the structure of the capsules prevents cellular material from being located on the surface of the capsule. Additionally, the structure of the capsules ensures that adequate gas exchange occurs with the cells and nutrients are received by the cells encapsulated therein. Optionally, the capsules also contain one or more anti-inflammatory drugs encapsulated therein for controlled release.

The disclosed compositions are formed from a biocompatible, hydrogel-forming polymer encapsulating the cells to be transplanted. Examples of materials which can be used to form a suitable hydrogel include polysaccharides such as alginate, collagen, chitosan, sodium cellulose sulfate, gelatin and agarose, water soluble polyacrylates, polyphosphazines, poly(acrylic acids), poly(methacrylic acids), poly(alkylene oxides), poly(vinyl acetate), polyvinylpyrrolidone (PVP), and copolymers and blends of each. See, for example, U.S. Pat. Nos. 5,709,854, 6,129,761, 6,858,229, and 9,555,007.

### VI. Pharmaceutical Compositions

As used herein the term “pharmaceutically acceptable” and “physiologically acceptable” mean a biologically acceptable composition, formulation, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Such composition, “pharmaceutically acceptable” and “physiologically acceptable” formulations and compositions can be sterile. Such pharmaceutical formulations and compositions may be used, for example in administering a viral particle or nanoparticle to a subject.

Such formulations and compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Supplementary active compounds (e.g., preservatives, antibacterial, antiviral and antifungal agents) can also be incorporated into the formulations and compositions.

Pharmaceutical compositions typically contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as surfactants, wetting or emulsifying agents, pH buffering substances, and the like, may be present in such vehicles.

Pharmaceutical compositions can be formulated to be compatible with a particular route of administration or delivery, as set forth herein or known to one of skill in the art. Thus, pharmaceutical compositions include carriers, diluents, or excipients suitable for administration or delivery by various routes.

Pharmaceutical forms suitable for injection or infusion of viral particles or nanoparticles can include sterile aqueous solutions or dispersions which are adapted for the extemporaneous preparation of sterile injectable or infusible solutions or dispersions, optionally encapsulated in liposomes. In all cases, the ultimate form should be a sterile fluid and stable under the conditions of manufacture, use and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Isotonic agents, for example, sugars, buffers or salts (e.g., sodium chloride) can be included. Prolonged absorption of injectable compositions can be brought about by the use in the compositions of agents delaying absorption, for example, aluminum monostearate and gelatin.

Solutions or suspensions of viral particles or nanoparticles can optionally include one or more of the following components: a sterile diluent such as water for injection, saline solution, such as phosphate buffered saline (PBS), artificial CSF, a surfactants, fixed oils, a polyol (for example, glycerol, propylene glycol, and liquid polyethylene glycol, and the like), glycerin, or other synthetic solvents; antibacterial and antifungal agents such as parabens, chlorobutanol, phenol, ascorbic acid, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

Pharmaceutical formulations, compositions and delivery systems appropriate for the compositions, methods and uses of the invention are known in the art (see, e.g., Remington: The Science and Practice of Pharmacy (2003) 20^{th} ed., Mack Publishing Co., Easton, PA; Remington’s Pharmaceutical Sciences (1990) 18^{th} ed., Mack Publishing Co., Easton, PA; The Merck Index (1996) 12^{th} ed., Merck Publishing Group, Whitehouse, NJ; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, MD; and Poznansky et al., Drug Delivery Systems (1980), R. L. Juliano, ed., Oxford, N.Y., pp. 253-315).

Viral particles, nanoparticles, and their compositions may be formulated in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms are dependent upon the number of viral particles or nanoparticles believed necessary to produce the desired effect(s). The amount necessary can be formulated in a single dose, or can be formulated in multiple dosage units. The dose may be adjusted to a suitable viral particle or nanoparticle concentration, optionally combined with an anti-inflammatory agent, and packaged for use.

In one embodiment, pharmaceutical compositions will include sufficient genetic material to provide a therapeutically effective amount, i.e., an amount sufficient to reduce or ameliorate symptoms or an adverse effect of a disease state in question or an amount sufficient to confer the desired benefit.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Thus, for example, viral particles, nanoparticles, and pharmaceutical compositions thereof can be packaged in single or multiple unit dosage form for ease of administration and uniformity of dosage.

Formulations containing viral particles or nanoparticles typically contain an effective amount, the effective amount being readily determined by one skilled in the art. The viral particles or nanoparticles may typically range from about 1% to about 95% (w/w) of the composition, or even higher if suitable. The quantity to be administered depends upon factors such as the age, weight and physical condition of the mammal or the human subject considered for treatment. Effective dosages can be established by one of ordinary skill in the art through routine trials establishing dose response curves.

### VII. Definitions

The terms “polynucleotide,” “nucleic acid” and “transgene” are used interchangeably herein to refer to all forms of nucleic acid, oligonucleotides, including deoxyribonucleic acid (DNA) and ribonucleic acid (RNA) and polymers thereof. Polynucleotides include genomic DNA, cDNA and antisense DNA, and spliced or unspliced mRNA, rRNA, tRNA and inhibitory DNA or RNA (RNAi, e.g., small or short hairpin (sh)RNA, microRNA (miRNA), small or short interfering (si)RNA, trans-splicing RNA, or antisense RNA). Polynucleotides can include naturally occurring, synthetic, and intentionally modified or altered polynucleotides (e.g., variant nucleic acid). Polynucleotides can be single stranded, double stranded, or triplex, linear or circular, and can be of any suitable length. In discussing polynucleotides, a sequence or structure of a particular polynucleotide may be described herein according to the convention of providing the sequence in the 5′ to 3′ direction.

A nucleic acid encoding a polypeptide often comprises an open reading frame that encodes the polypeptide. Unless otherwise indicated, a particular nucleic acid sequence also includes degenerate codon substitutions.

Nucleic acids can include one or more expression control or regulatory elements operably linked to the open reading frame, where the one or more regulatory elements are configured to direct the transcription and translation of the polypeptide encoded by the open reading frame in a mammalian cell. Non-limiting examples of expression control/regulatory elements include transcription initiation sequences (e.g., promoters, enhancers, a TATA box, and the like), translation initiation sequences, mRNA stability sequences, poly A sequences, secretory sequences, and the like. Expression control/regulatory elements can be obtained from the genome of any suitable organism.

A “promoter” refers to a nucleotide sequence, usually upstream (5′) of a coding sequence, which directs and/or controls the expression of the coding sequence by providing the recognition for RNA polymerase and other factors required for proper transcription. “Promoter” includes a minimal promoter that is a short DNA sequence comprised of a TATA-box and optionally other sequences that serve to specify the site of transcription initiation, to which regulatory elements are added for control of expression.

An “enhancer” is a DNA sequence that can stimulate transcription activity and may be an innate element of the promoter or a heterologous element that enhances the level or tissue specificity of expression. It is capable of operating in either orientation (5′->3′ or 3′->5′), and may be capable of functioning even when positioned either upstream or downstream of the promoter.

Promoters and/or enhancers may be derived in their entirety from a native gene, or be composed of different elements derived from different elements found in nature, or even be comprised of synthetic DNA segments. A promoter or enhancer may comprise DNA sequences that are involved in the binding of protein factors that modulate/control effectiveness of transcription initiation in response to stimuli, physiological or developmental conditions.

Non-limiting examples include SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from non-viral genes, such as the murine metallothionein gene, will also find use herein. Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

A “transgene” is used herein to conveniently refer to a nucleic acid sequence/polynucleotide that is intended or has been introduced into a cell or organism. Transgenes include any nucleic acid, such as a gene that encodes an inhibitory RNA or polypeptide or protein, and are generally heterologous with respect to naturally occurring AAV genomic sequences.

The term “transduce” refers to introduction of a nucleic acid sequence into a cell or host organism by way of a vector (e.g., a viral particle). Introduction of a transgene into a cell by a viral particle is can therefore be referred to as “transduction” of the cell. The transgene may or may not be integrated into genomic nucleic acid of a transduced cell. If an introduced transgene becomes integrated into the nucleic acid (genomic DNA) of the recipient cell or organism it can be stably maintained in that cell or organism and further passed on to or inherited by progeny cells or organisms of the recipient cell or organism. Finally, the introduced transgene may exist in the recipient cell or host organism extra chromosomally, or only transiently. A “transduced cell” is therefore a cell into which the transgene has been introduced by way of transduction. Thus, a “transduced” cell is a cell into which, or a progeny thereof in which a transgene has been introduced. A transduced cell can be propagated, transgene transcribed and the encoded inhibitory RNA or protein expressed. For gene therapy uses and methods, a transduced cell can be in a mammal.

Transgenes under control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting a suitable promoter (constitutive versus inducible; strong versus weak), it is possible to control both the existence and level of expression of a polypeptide in the genetically modified cell. If the gene encoding the polypeptide is under the control of an inducible promoter, delivery of the polypeptide in situ is triggered by exposing the genetically modified cell in situ to conditions for permitting transcription of the polypeptide, e.g., by intraperitoneal injection of specific inducers of the inducible promoters which control transcription of the agent. For example, in situ expression by genetically modified cells of a polypeptide encoded by a gene under the control of the metallothionein promoter, is enhanced by contacting the genetically modified cells with a solution containing the appropriate (i.e., inducing) metal ions in situ.

A nucleic acid/transgene is “operably linked” when it is placed into a functional relationship with another nucleic acid sequence. A nucleic acid/transgene encoding and RNAi or a polypeptide, or a nucleic acid directing expression of a polypeptide may include an inducible promoter, or a tissue-specific promoter for controlling transcription of the encoded polypeptide. A nucleic acid operably linked to an expression control element can also be referred to as an expression cassette.

In certain embodiments, CNS-specific or inducible promoters, enhancers and the like, are employed in the methods and uses described herein. Non-limiting examples of CNS-specific promoters include those isolated from the genes from myelin basic protein (MBP), glial fibrillary acid protein (GFAP), and neuron specific enolase (NSE). Non-limiting examples of inducible promoters include DNA responsive elements for ecdysone, tetracycline, hypoxia and IFN.

In certain embodiments, an expression control element comprises a CMV enhancer. In certain embodiments, an expression control element comprises a beta actin promoter. In certain embodiments, an expression control element comprises a chicken beta actin promoter. In certain embodiments, an expression control element comprises a CMV enhancer and a chicken beta actin promoter.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular type of variant is a mutant protein, which refers to a protein encoded by a gene having a mutation, e.g., a missense or nonsense mutation.

A “nucleic acid” or “polynucleotide” variant refers to a modified sequence which has been genetically altered compared to wild-type. The sequence may be genetically modified without altering the encoded protein sequence. Alternatively, the sequence may be genetically modified to encode a variant protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of a protein encoded thereby, and some codons of the nucleic acid variant will be changed which in turn changes the amino acids of a protein encoded thereby.

The terms “protein” and “polypeptide” are used interchangeably herein. The “polypeptides” encoded by a “nucleic acid” or “polynucleotide” or “transgene” disclosed herein include partial or full-length native sequences, as with naturally occurring wild-type and functional polymorphic proteins, functional subsequences (fragments) thereof, and sequence variants thereof, so long as the polypeptide retains some degree of function or activity. Accordingly, in methods and uses of the invention, such polypeptides encoded by nucleic acid sequences are not required to be identical to the endogenous protein that is defective, or whose activity, function, or expression is insufficient, deficient or absent in a treated mammal.

Non-limiting examples of modifications include one or more nucleotide or amino acid substitutions (e.g., about 1 to about 3, about 3 to about 5, about 5 to about 10, about 10 to about 15, about 15 to about 20, about 20 to about 25, about 25 to about 30, about 30 to about 40, about 40 to about 50, about 50 to about 100, about 100 to about 150, about 150 to about 200, about 200 to about 250, about 250 to about 500, about 500 to about 750, about 750 to about 1000 or more nucleotides or residues).

An example of an amino acid modification is a conservative amino acid substitution or a deletion. In particular embodiments, a modified or variant sequence retains at least part of a function or activity of the unmodified sequence (e.g., wild-type sequence).

Another example of an amino acid modification is a targeting peptide introduced into a capsid protein of a viral particle. Peptides have been identified that target recombinant viral vectors or nanoparticles, to the central nervous system, such as vascular endothelial cells. Thus, for example, endothelial cells lining brain blood vessels can be targeted by the modified recombinant viral particles or nanoparticles.

A recombinant virus so modified may preferentially bind to one type of tissue (e.g., CNS tissue) over another type of tissue (e.g., liver tissue). In certain embodiments, a recombinant virus bearing a modified capsid protein may “target” brain vascular epithelia tissue by binding at level higher than a comparable, unmodified capsid protein. For example, a recombinant virus having a modified capsid protein may bind to brain vascular epithelia tissue at a level 50% to 100% greater than an unmodified recombinant virus.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein. In certain embodiments, the fragment or portion is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. In certain embodiments, the variant is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

“Conservative variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

The term “substantial identity” in the context of a polypeptide indicates that a polypeptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. An indication that two polypeptide sequences are identical is that one polypeptide is immunologically reactive with antibodies raised against the second polypeptide. Thus, a polypeptide is identical to a second polypeptide, for example, where the two peptides differ only by a conservative substitution.

The terms “treat” and “treatment” refer to both therapeutic treatment and prophylactic or preventative measures, wherein the object is to prevent, inhibit, reduce, or decrease an undesired physiological change or disorder, such as the development, progression or worsening of the disorder. For purposes of this invention, beneficial or desired clinical results include, but are not limited to, alleviation of symptoms, diminishment of extent of disease, stabilizing a (i.e., not worsening or progressing) symptom or adverse effect of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those predisposed (e.g., as determined by a genetic assay).

The terms “comprising,” “having,” “including,” and “containing” are to be construed as open-ended terms (i.e., meaning “including, but not limited to”) unless otherwise noted.

All methods and uses described herein can be performed in any suitable order unless otherwise indicated herein or otherwise clearly contradicted by context. The use of any and all examples, or exemplary language (e.g., “such as” or “for example”) provided herein, is intended merely to better illuminate the invention and does not pose a limitation on the scope of the invention unless otherwise claimed. No language in the specification should be construed as indicating any non-claimed element as essential to the practice of the invention.

All of the features disclosed herein may be combined in any combination. Each feature disclosed in the specification may be replaced by an alternative feature serving a same, equivalent, or similar purpose. Thus, unless expressly stated otherwise, disclosed features (e.g., modified nucleic acid, vector, plasmid, a recombinant vector sequence, vector genome, or viral particle) are an example of a genus of equivalent or similar features.

As used herein, the forms “a”, “and,” and “the” include singular and plural referents unless the context clearly indicates otherwise. Thus, for example, reference to “a nucleic acid” includes a plurality of such nucleic acids, reference to “a vector” includes a plurality of such vectors, and reference to “a virus” or “AAV or rAAV particle” includes a plurality of such virions/AAV or rAAV particles.

The term “about” at used herein refers to a values that is within 10% (plus or minus) of a reference value.

Recitation of ranges of values herein are merely intended to serve as a shorthand method of referring individually to each separate value falling within the range, unless otherwise indicated herein, and each separate value is incorporated into the specification as if it were individually recited herein.

Accordingly, all numerical values or numerical ranges include integers within such ranges and fractions of the values or the integers within ranges unless the context clearly indicates otherwise. Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-20, 10-50, 30-50, 50-100, 100-300, 100-1,000, 1,000-3,000, 2,000-4,000, 4,000-6,000, etc.

### VIII. Kits

The invention provides kits with packaging material and one or more components therein. A kit typically includes a label or packaging insert including a description of the components or instructions for use in vitro, in vivo, or ex vivo, of the components therein. A kit can contain a collection of such components, e.g., a nucleic acid, recombinant vector, viral particles, splicing modifier molecules, and optionally a second active agent, such as another compound, agent, drug or composition.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a disease for which a kit component may be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, or an electrical storage media such as RAM and ROM or hybrids of these such as magnetic/optical storage media, FLASH memory, hybrids and memory type cards.

### IX. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1 - Materials and Methods

Cell culture, transfection, and LMI070/RG7800 treatment. Human embryonic kidney (HEK293) cells (obtained from CHOP Research Vector Core stock) were maintained in DMEM media containing 10% Fetal Bovine Serum (FBS), 1% L-Glutamine and 1% penicillin/streptomycin at 37° C. with 5% CO₂. Cells were cultured in 24 well plates and transfected at 80-90% confluence using Lipofectamine 2000 transfection reagent, according to the manufacturer’s protocol. For all experiments, 4 hours after plasmid transfection, cells were treated with LMI070 (MedChemExpress, HY-19620, suspended in DMSO) or RG7800 (MedChemExpress, HY-101792A, suspended in H₂O) at the indicated concentrations. Cells were tested for mycoplasma by Research Vector Core. None of the cells used in the study were listed in ICLAC database of commonly misidentified cell lines.

Plasmids, primers and custom made TaqMan gene expression assays. All plasmids, primer sequences, and custom Taqman gene expression assays to determine SF3B3 novel exon inclusion are available upon request. Primers and custom Taqman gene expression assays were obtained from IDT Integrated DNA Technologies.

In vitro luciferase assays. HEK293 cells were cultured in DMEM (10% FBS (v/v), 1% Pen/ Strep (v/v), and 1% L-glutamine (v/v) in a 24-well plate. At 70%-80% confluence, cells were co-transfected with the Xon.Firefly luciferase cassettes (0.3 µg/well) and a SV40p-Renilla luciferase cassette as transfection control (0.02 µg/well). Four hours after transfections cells were treated either with LMI070 or RG7800 at indicated concentrations. At 24 h after transfection, cells were rinsed with ice-cold PBS and Renilla and Firefly luciferase activities were assessed using the Dual-Luciferase Reporter Assay System (Promega) according to the manufacturer’s instructions. Luminescent readouts were obtained with a Monolight 3010 luminometer (Pharmigen). Relative light units (RLUs) were calculated as the quotient of Renilla/Firefly RLUs and results expressed relative to mock treated control cells.

Animals and histology. Animal protocols were approved by The Children’s Hospital of Philadelphia Institutional Animal Care and Use Committee. Five to six-week-old male C57Bl6/j mice were obtained from Jackson Laboratories (Bar Harbor, ME, USA). AAV vectors (AAV9. X^{on}.eGFP or AAVPHBeB.X^{on}.eGFP; generated at the CHOP Research Vector Core) were administrated by retroorbital injection at 7-8 weeks of age with a total of 3E11 vg in 150 µL infused. After 4 weeks, a single dose of LMI070 (5 or 50 mg/kg, MedChemExpress HY-19620) or vehicle solution was administrated by oral gavage. After 18-24 h, mice used for biochemical or molecular studies were anesthetized and perfused with 0.9% cold saline mixed with 2-ml RNAlater (Ambion) solution. Brains and liver samples were collected, flash frozen in liquid nitrogen, and stored at -80 C until use. For immunohistochemistry studies, mice were perfused with 15 mL ice-cold 0.1 M PBS followed by 15 mL 4% paraformaldehyde. For brain sections, eGFP visualization was done by IHC using rabbit anti-GFP antibody (Invitrogen, 1:200) followed by Alexa488-conjugated goat anti-rabbit (Invitrogen, 1:500) and Alexa488-conjugated chicken anti-goat (Invitrogen, 1:500). Slices were mounted on Superfrost plus slides and coverslips mounted using fluoro-gel mounting media. Sections were analyzed using a DM6000B Leica microscope equipped with a L5 ET filter cube (ex:em of 470±20:525±15 nm and dichroic 495 nm), a 20X HC APO PLAN (N.A. 0.70) lens connected to a Sola Light Engine LED light source (Lumencor). Images were collected with a Hammatsu Orca flash4.0 monochrome camera controlled by Leica LAS X (v.3.0.3) software. Brain images represent a 7.98 µm thick z-stack deconvoluted by 3 iterations of the Blind algorithm.

RNA extraction, RT-PCR, and Splicing assays. Total RNA was extracted using Trizol (Life Technologies) according to the manufacturer’s protocol, with the exception of 1 µL Glycoblue (Life Technologies) in addition to the aqueous phase on the isopropanol precipitation step and a single wash with cold 70% ethanol. To determine HTT expression levels after transfection, RNA samples were quantified by spectrophotometry and subsequently cDNAs generated from 1 mg of total RNA with random hexamers (TaqMan RT reagents, Applied Biosystems). To determine human HTT expression levels in HEK293 cells, we used TaqMan probes for human HTT and glyceraldehyde 3-phosphate dehydrogenase (GAPDH) mRNAs obtained from Applied Biosystems. Relative HTT gene expression was determined using the ddCt method. To determine splicing of the SMN2-on and Xon switches, 2 µg of total RNA from HEK293 cells or tissue samples was treated with DNAseI Free kit (Thermofisher) followed by cDNA generation using the High capacity cDNA kit (Thermofisher). Splicing was determined by PCR using the Phusion High-Fidelity polymerase (Thermofisher) and PCR products separated on a 2.5% agarose gel pre-stained with EtBr and spliced-in and spliced-out band densitometry performed using with the ChemiDoc Imaging System (BioRad) and Image Lab analysis software. Splicing induction from mouse tissues was determined using two custom TaqMan assays designed to determine total or LMI070-spliced in mRNA transcripts. The percentage of induction was determined by dividing the average Ct novel exon and average Ct Total, relative to control animals injected with AAV virus plus vehicle.

Western Blots. HEK293 cells were rinsed and lysed with Passive Lysis Buffer (PBL, Promega), protein concentrations determined using the DC protein assay (Bio-Rad), and 10-20 µg of protein loaded on a 3%-8% NuPAGE Tris-Acetate Gel in tris-acetate buffer (Novex Life Technologies) or a 4-12% NuPAGE TrisBis NuPAGE gels in MES buffer (Novex Life Technologies) to determine HTT, SaCas9, or eGFP protein levels, respectively, with β-catenin as loading control. Livers were homogenized in RIPA buffer (final concentration: 50 mM Tris, 150 mM NaCl, 1% Triton-X100, 0.1% SDS, 0.5% sodium deoxycholate, with Complete protease inhibitors (Roche) and samples incubated for 1 hr rotating at 4° C. then clarified by centrifugation at 10,000 x g for 10 minutes. Total protein concentration was determined by DC protein assay (BioRad) and 30 µg loaded on a 4-12% NuPAGE Bis-tris gels in MES buffer (Novex Life Technologies) to determine eGFP and β-catenin levels. After electrophoresis, proteins were transferred to 0.2 µm PVDF (Bio-Rad). Membranes were blocked with 5% milk in PBS-T and then blotted with a mouse anti-HTT (MAB2166. dilution: 1:5,000; Millipore), rabbit anti β-catenin (Ab2365. dilution: 1:5,000; Abcam), HA Tag antibody for SaCas9 protein (2-2.2.14, Thermofisher), rabbit anti-GFP (A11122, Invitrogen) followed by horseradish peroxidase-coupled antibodies (Goat antimouse: 115-035-146. dilution: 1:10,000 or Goat anti-Rabbit: 111-035-144. dilution: 1:50,000; Jackson ImmunoResearch). Blots were developed with ECL Plus reagents (Amersham Pharmacia) and imaged on the ChemiDoc Imaging System (BioRad).

RNA-Seq Methods. Data from 4 LMI070 treated and 4 DMSO treated HEK293 cell groups were obtained after sequencing across two lanes run on an Illumina Hi-Seq 4000. The resulting fastq files were aligned to the GRCh38 human genome obtained from Ensembl using the STAR aligner (Dobin & Gingeras, 2015). Splice junction output by STAR were quantified using a custom R script designed to identify splicing events unique to LMI070 treatment. Top ranking LMI070-exclusive splice events were manually assessed for their applicability to function as a splicing switch. A primary requirement of this evaluation was that two splice events (donor and acceptor) were identified as exclusive or enriched in LMI070 treated cells creating inclusion of a pseudoexon of reasonable size. Candidate splice events were visually evaluated using the Sashimi plot function available in IGV (Thorvaldsdottir et al., 2013; Katz et al., 2015).

To assess the exclusivity of LMI070 induced splice sites to LMI070 treatment, the frequency with which candidate splice junctions were previously identified in diverse human RNA-Seq datasets deposited in the sequence read archive (SRA) was evaluated. This analysis was performed using Intropolis, a database of exon-exon junctions from 21,504 human RNA-Seq samples in the SRA archive. The Intropolis database is indexed by GRCh37 genomic position so the GRCh38 positions were first converted to GRCh37 using the LiftOver tool from the UCSC genome browser (Kent et al., 2002). Then, LMI070 induced splice sites were queried against the Intropolis database using a custom python script. The results for each LMI070 candidate splice event are summarized in Table 1.

Differential gene expression analysis was performed using DESeq2 to compare samples from the LMI070 and DMSO conditions (Love et al., 2014). To visualize the abundance of meaningfully differentially expressed genes, a volcano plot was generated with a .05 Benjamini-Hochberg adjusted p-value threshold and a 0.1 log fold change threshold.

Data availability. Custom R and Python scripts will be made available on Github. RNA-Seq datasets will be archived in the NCBI Gene Expression Omnibus (GEO).

Statistical analysis. Statistical analyses were performed using GraphPad Prism v5.0 software. Outlier samples were detected using the Grubb’s test (a = 0.05). Normal distribution of the samples was determined by using the D′Agostino and Pearson normality test. Data was analyzed using one-way ANOVA followed by a Bonferroni’s post hoc. Statistical significance was considered with a p < 0.05. All results are shown as the mean ± SEM.

### Example 2 - Regulated Control of Gene Therapies With a Drug-induced Switch

Initial experiments to test the present approach were done using the actual target of the drugs, the SMN2 gene, for Spinal Muscular Atrophy (SMA) therapy. SMA is due to mutations in the gene SMN1. Humans have a very similar gene, SMN2 that can serve to modify the severity of SMN1 deficiency depending on the number of SMN2 copies resident in the patient’s genome. SMN2 cannot fully replace SMN1, however, because unlike SMN1, SMN2 has undergone variations impairing exon 7 inclusion. As a consequence, only ~10% of SMN2 is correctly spliced (Cartegnie et al., 2006; Cartegni & Krainer, 2002). Two drugs, LMI070 (Cheung et al., 2018) and RG7800/RG7619 (Ratni et al., 2016), can improve exon 7 inclusion and are in later stage clinical testing. It was reasoned that the exon 6/7/8 cassette could be co-opted and refined to control the expression of a gene of interest, rather than SMN2 protein.

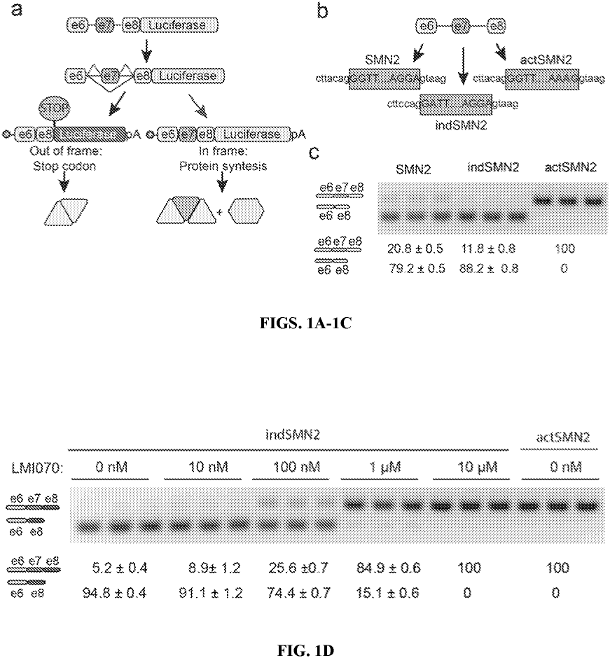

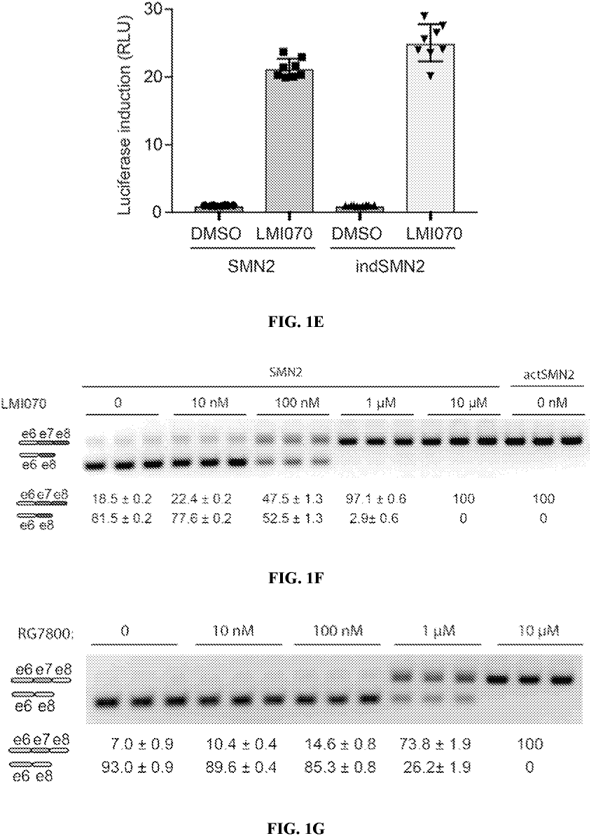

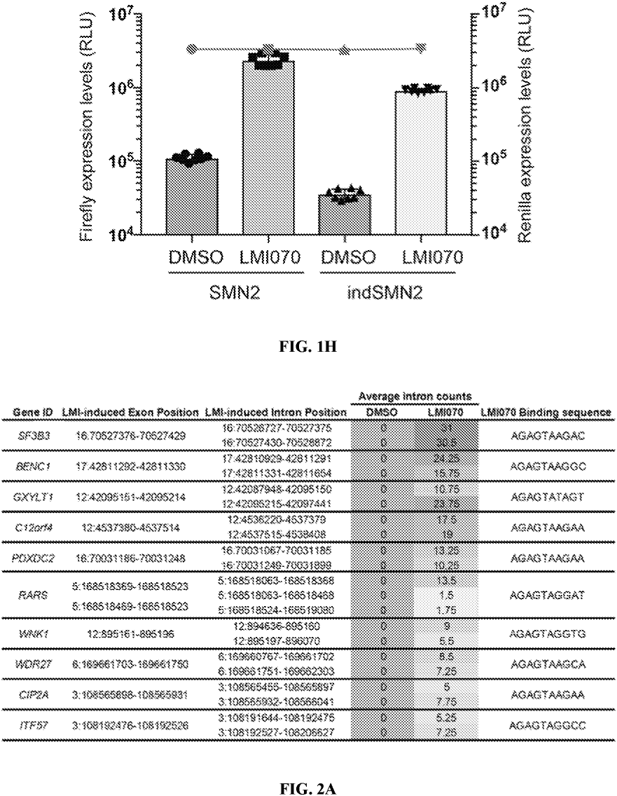

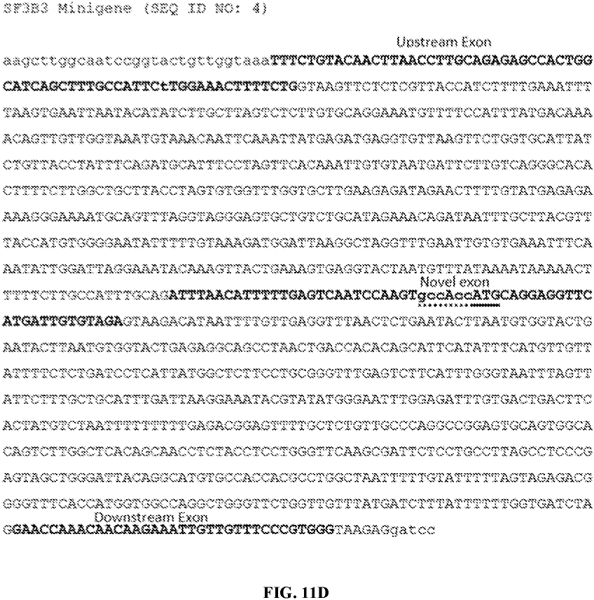

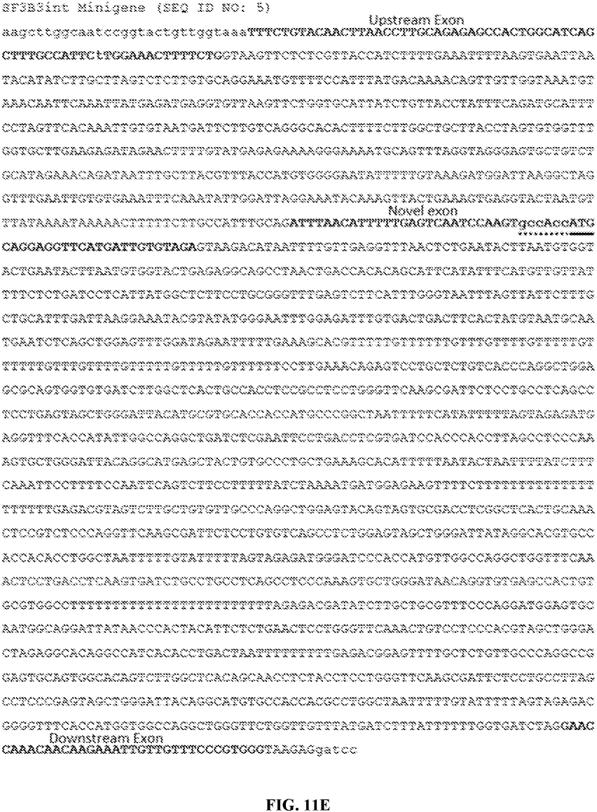

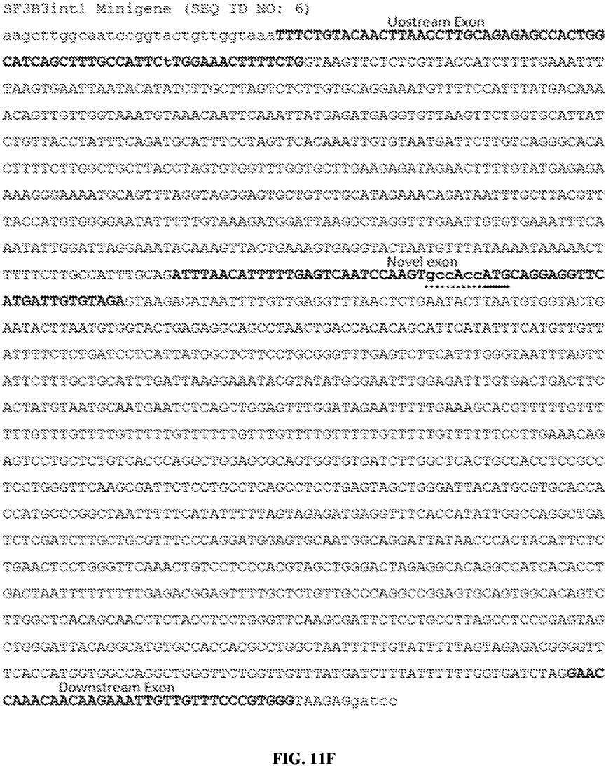

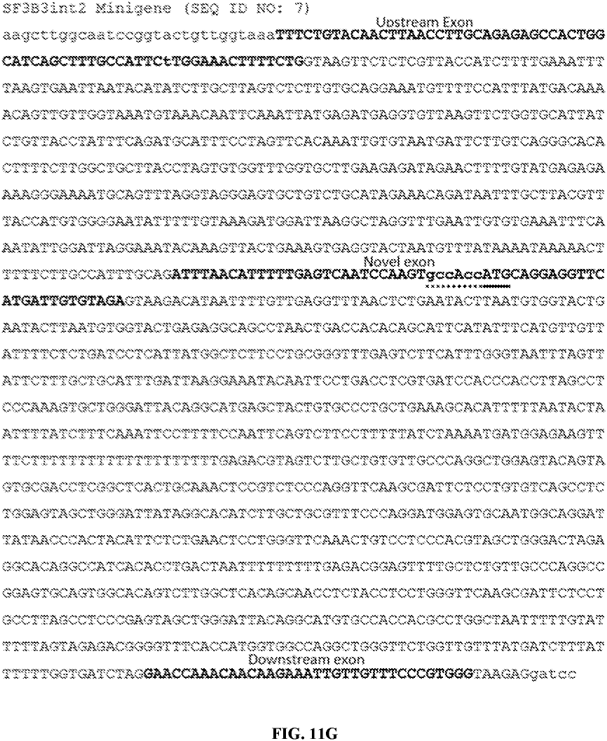

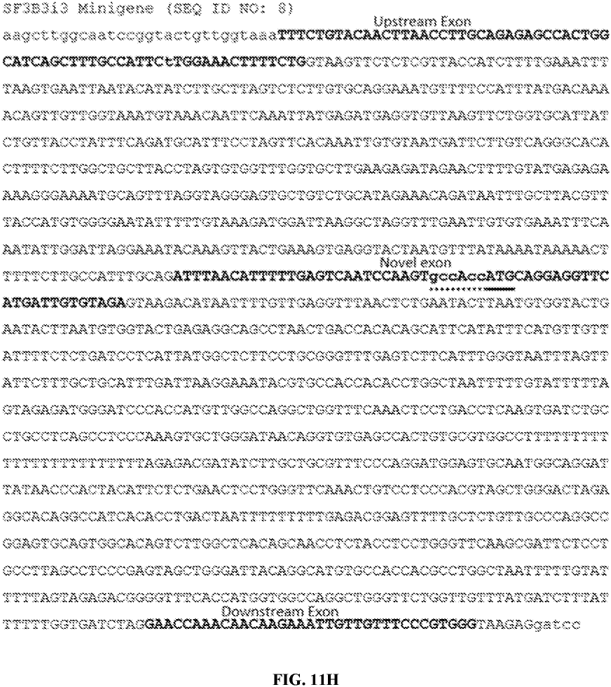

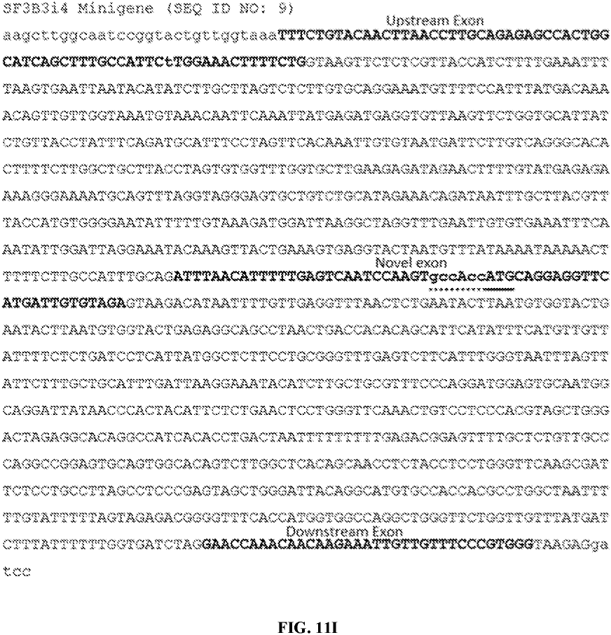

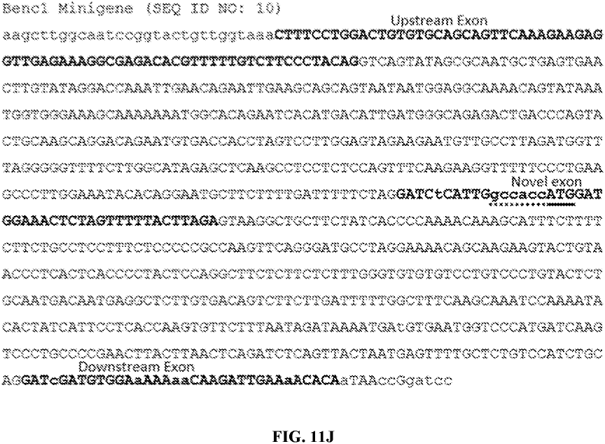

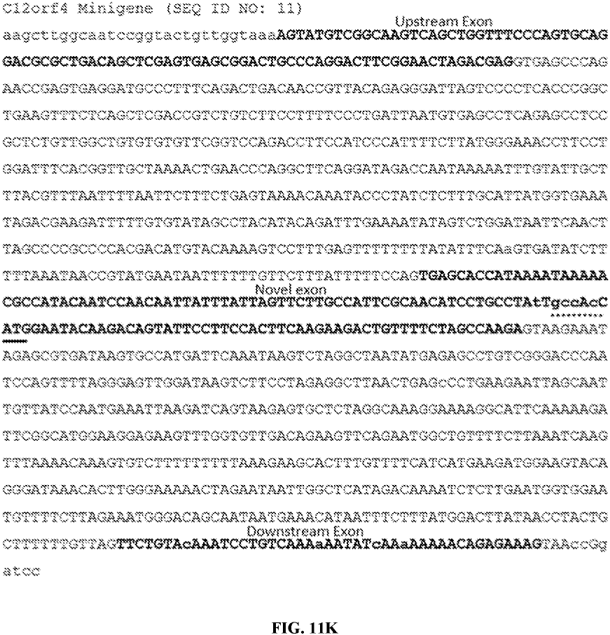

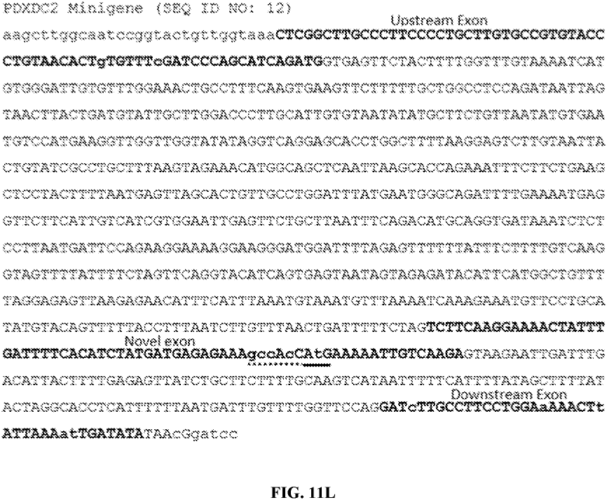

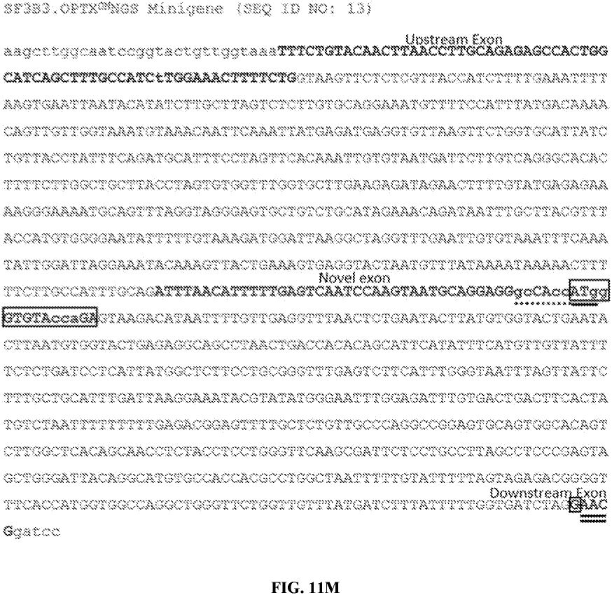

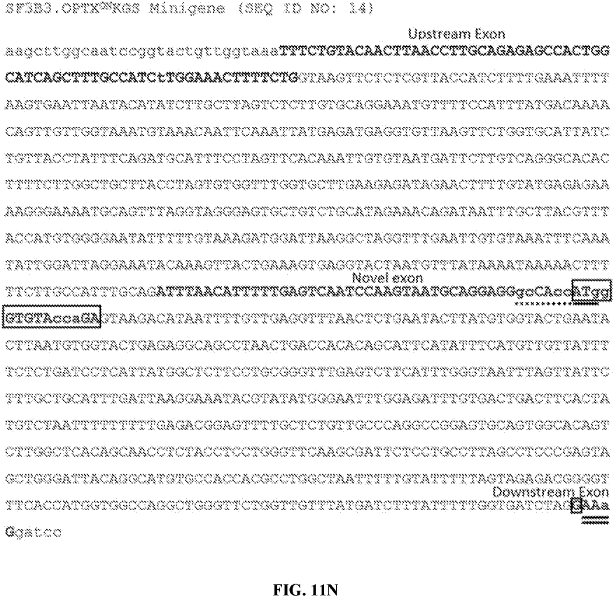

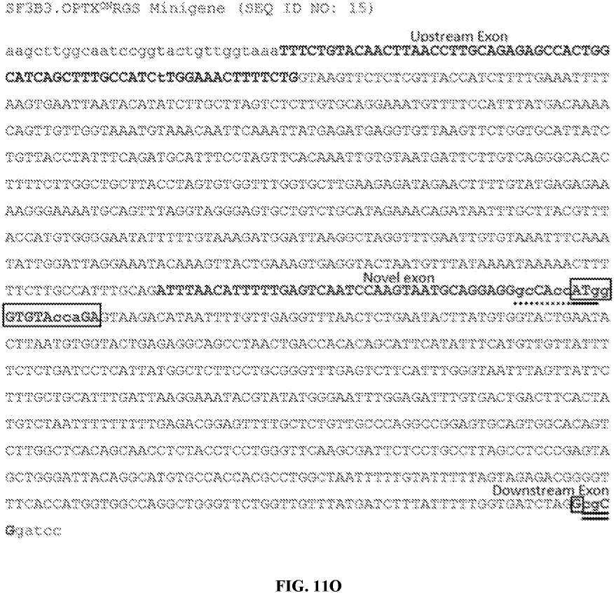

For this, SMN2-on cassettes were generated to provide drug-induced reporter gene expression. HEK293 cells were transfected with the SMN2-on expression cassettes and luciferase activity evaluated in response to varying doses of LMI070 or RG7800. As depicted in FIG. 1A, firefly luciferase activity would be expected with exon 7 inclusion, while exon 7 exclusion results in the presence of a premature stop codon and lack of signal. The SMN2-on switch was tested in its native format or altered for constitutive inclusion or reduced background exon 7 inclusion by modifying SMN2 exon 7 donor or acceptor splice sites, respectively (FIGS. 1B-1C and 11A-11C). Both RG7800 and LMI070 induced luciferase activity from the SMN2-on minigene, with a complete splicing switch evident at drug concentrations greater than 1 µM (FIGS. 1C, 1F, and 1G) and an overall induction of approximately 20-fold for the refined SMN2-on switch system (FIGS. 1D, 1E, and 1H). In the setting of the SMN2-on cassette, LMI070 was more active than RG7800 (FIGS. 1D, 1F, and 1G), which may be due to their different mechanisms of action (Palacino et al., 2015; Wang et al., 2018).

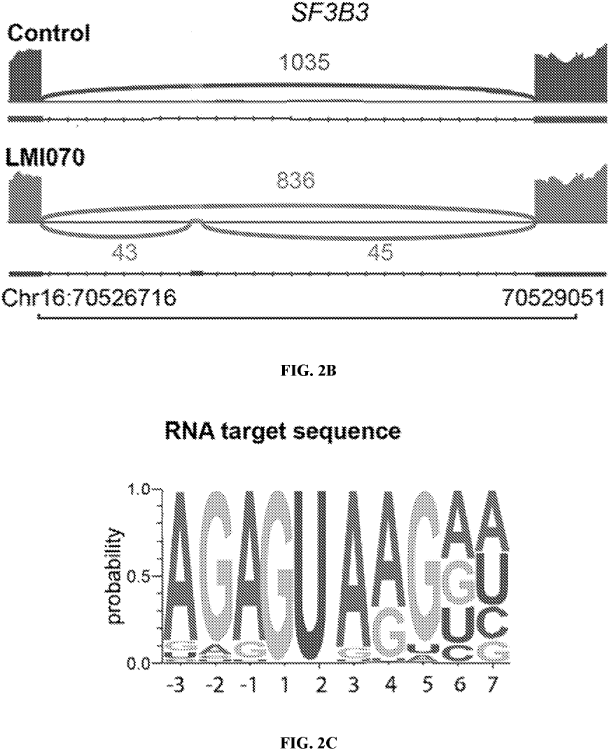

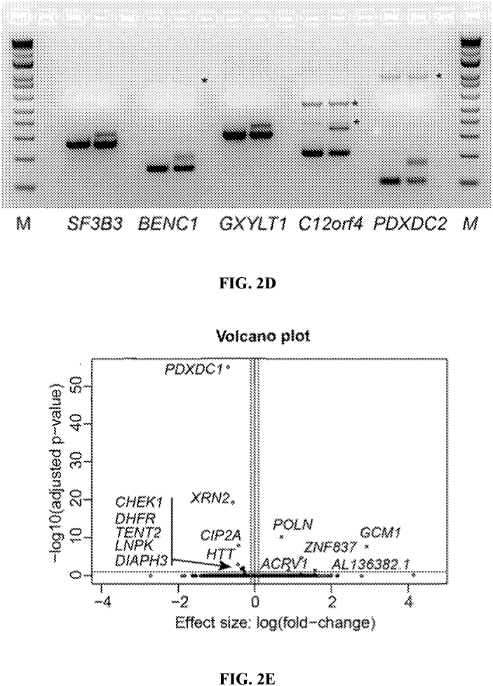

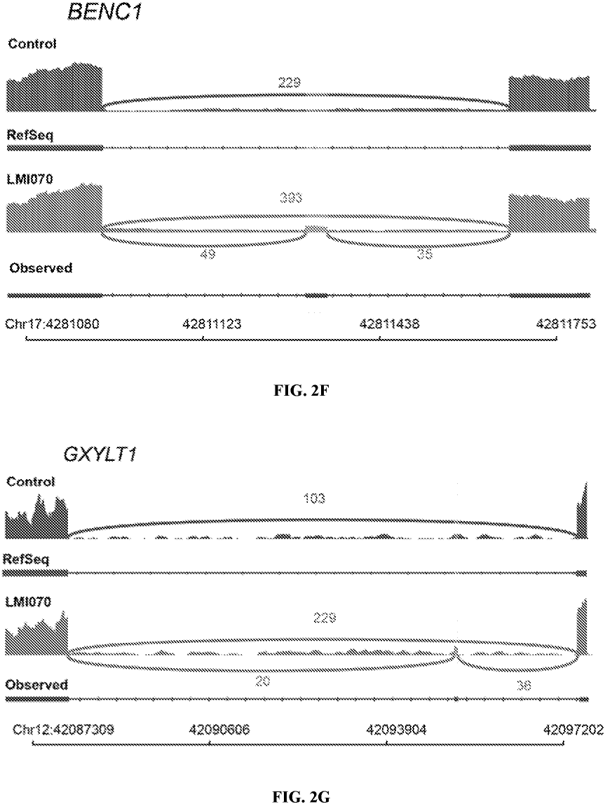

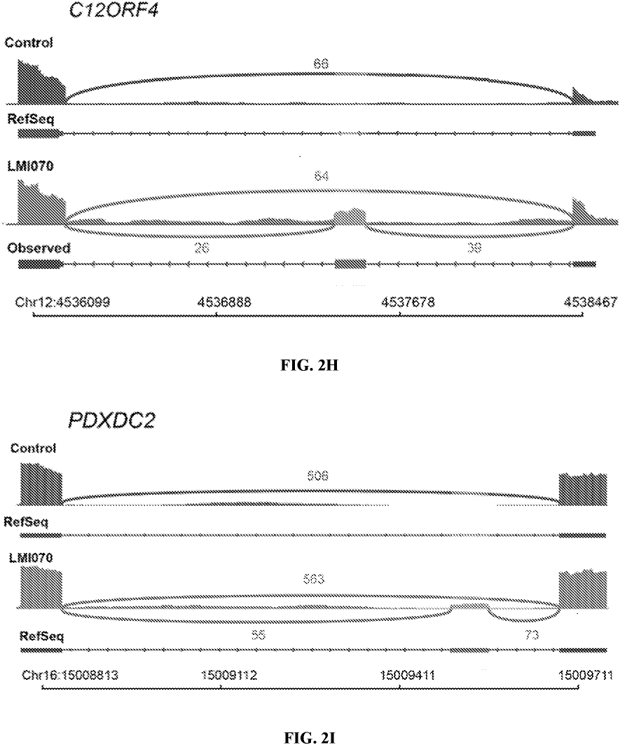

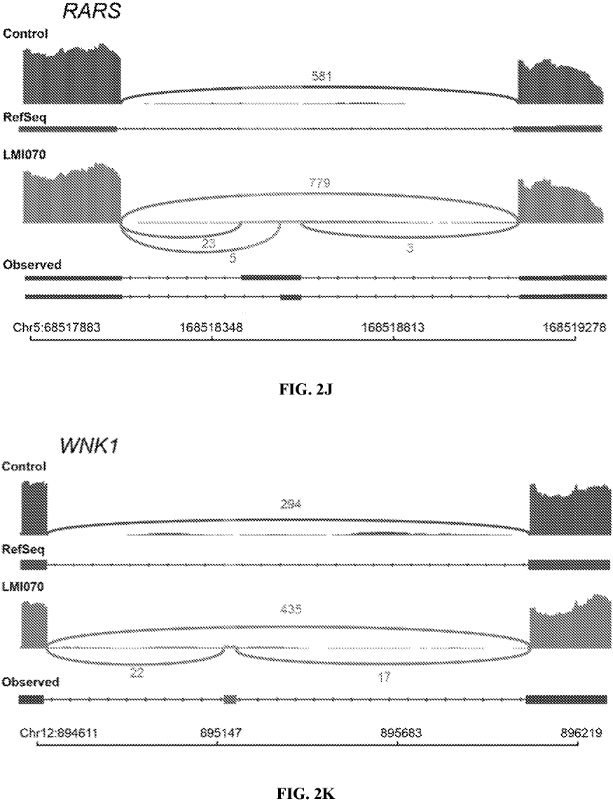

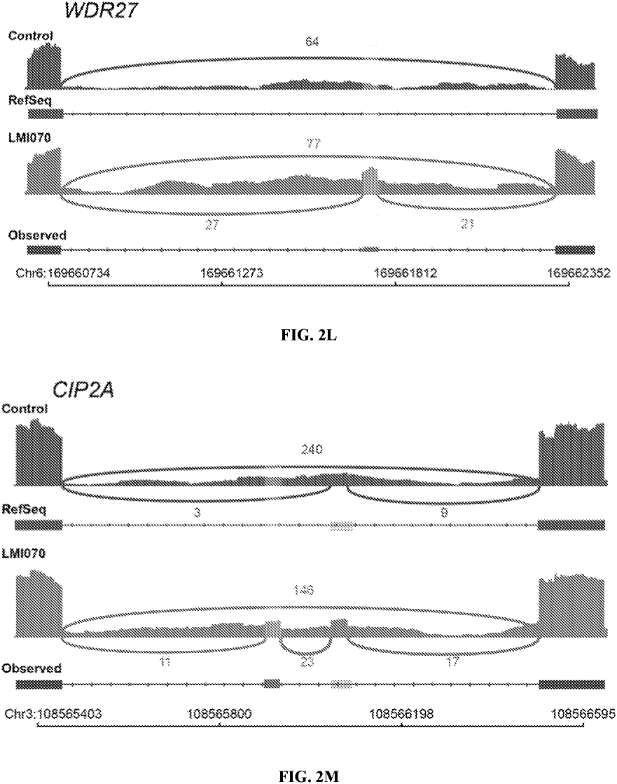

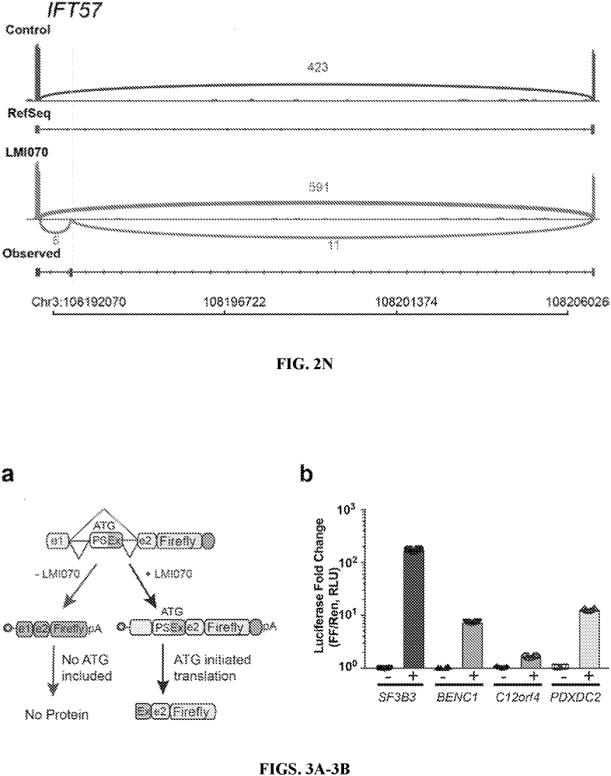

Next, exons that were more sensitive to LMI070 to reduce non-target splice events were sought out. HEK293 cells were treated with 25 nM LMI070 for 12 hours, and the splicing changes induced were ascertained using RNA-Seq. Reads obtained from 4 control and 4 LMI070 treated samples were aligned to the genome using the STAR aligner (Dobin & Gingeras, 2015) and splicing events exclusive to the LMI070 treated samples identified (FIGS. 2A and 2F-2N; Table 1). A total of 45 novel splicing events were identified following LMI070 treatment that were above the threshold of an average of greater than 5 novel intron splicing events in LMI070 treated samples (FIGS. 2A-2D; Table 1). Among them, 23 events were found exclusively and in all LMI070 treated samples, and the remaining 22 were evident in all treated and in one of the control untreated samples (Table 1). To assess exclusivity of the 45 identified candidate positions to LMI070 treatment the chromosomal locations were assessed in Intropolis (Nellore et al., 2016), a resource containing a list of all exon-exon junctions found in 21,504 human RNA-Seq datasets. In one example, the canonical exon-exon junction in SF3B3 (FIG. 2B; Table 1) was observed in 12,872 datasets at an average frequency of 64 counts per dataset, while the LMI070 induced splice event was observed in 10 and 1 dataset(s), respectively, for the 5′ and 3′ exon-exon junctions. The average counts per dataset for the 5′ exon-exon junction was 1.3 while the 3′ exon-exon junction was observed only once in all 21,504 sets (Table 1).

The pseudoexons identified in LMI070 treated samples share a strong 3′ AGAGUA motif consistent with the previously identified U1 RNA binding site targeted by LMI070 (FIG. 2C) (Cheung et al., 2018). To experimentally validate the identified LMI070 induced splicing events, primer pairs binding the flanking exons of the top 5 candidate genes (FIGS. 2A and 2F-2J) were generated. Robust amplification of the novel exons was detected by PCR exclusively on cDNA samples generated from HEK293 cells treated with LMI070 (FIG. 2D). The impact of LMI070 treatment on global gene expression was evaluated by assessing differential expression analysis. DESeq2 revealed strikingly few differentially expressed genes with only 6 upregulated and 24 downregulated genes passing the threshold for significance (p < 0.05; Benjamin-Hochberg multiple testing correction)⁹. After filtering out genes with low fold-change values (<0.1 fold), only 5 upregulated and 9 downregulated genes were identified (FIG. 2E).

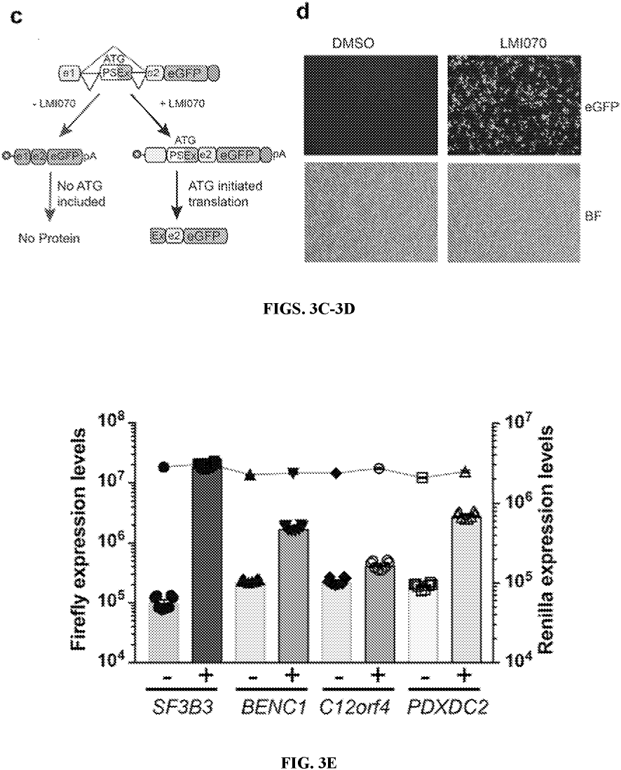

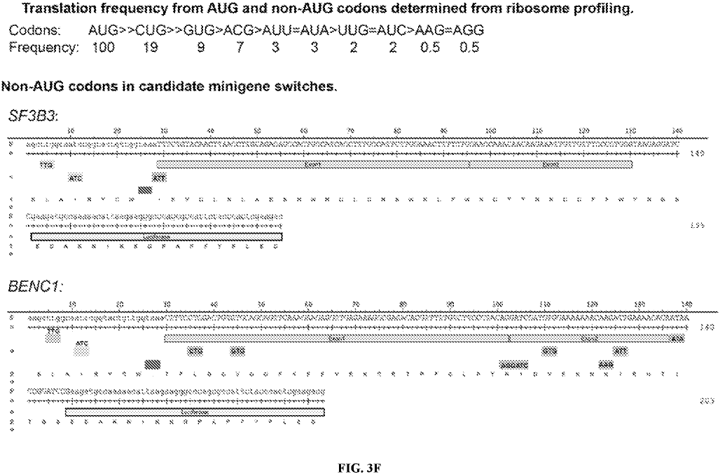

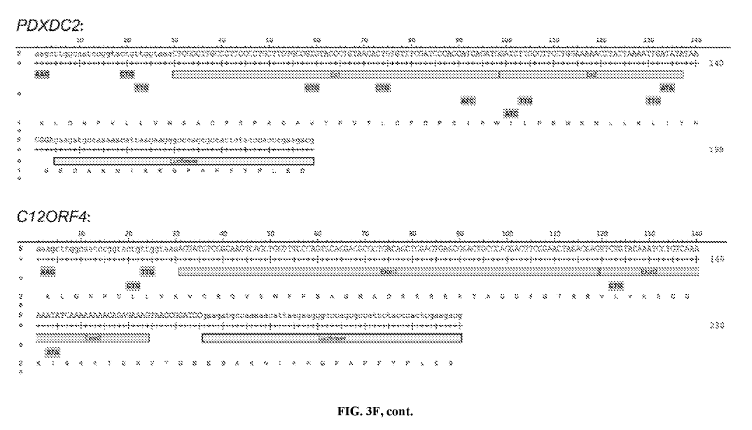

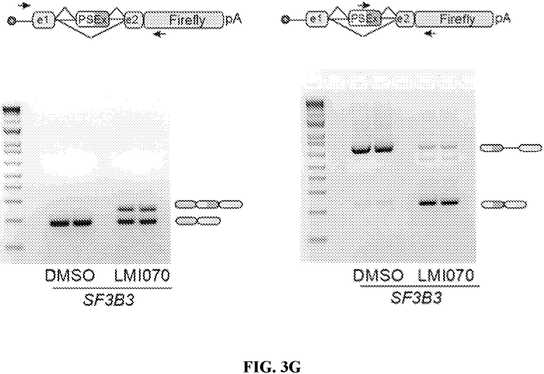

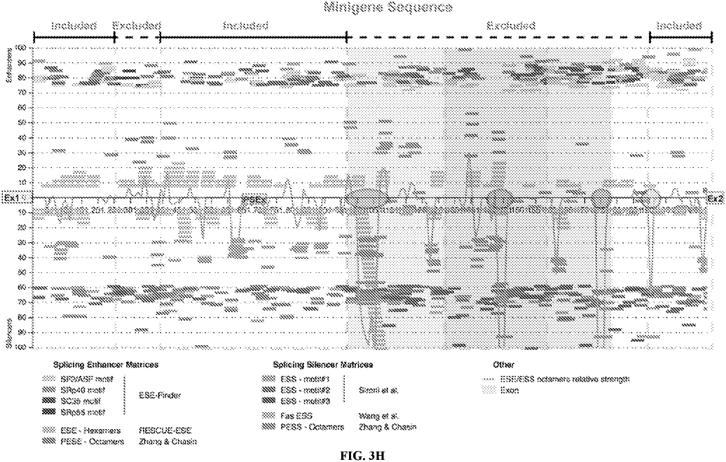

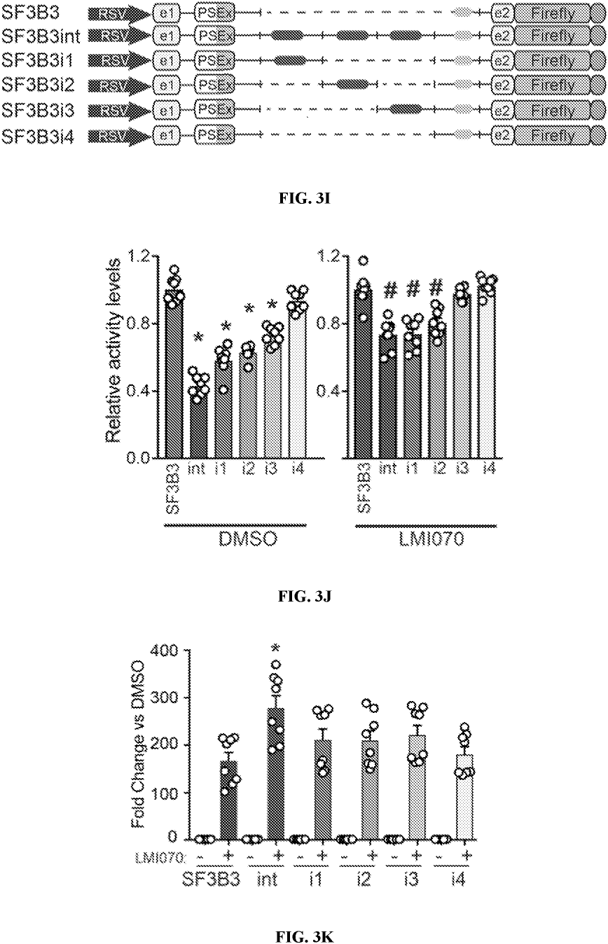

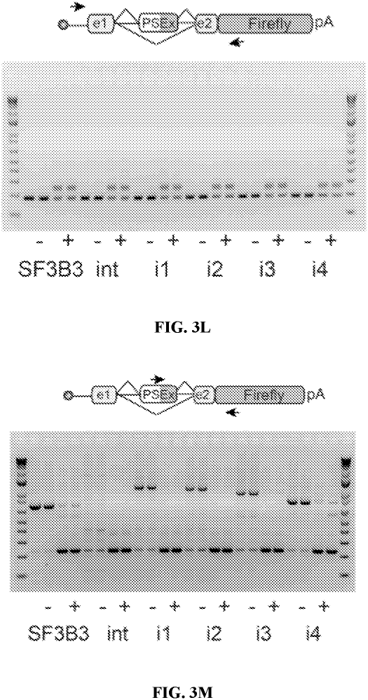

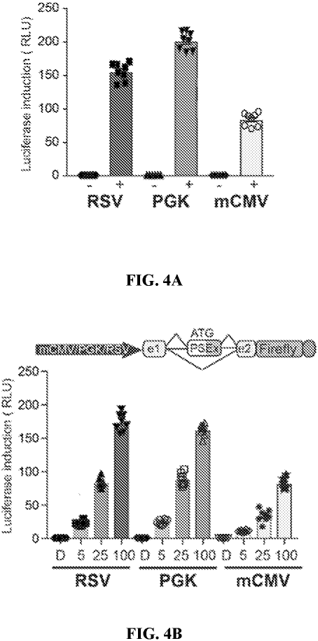

Next, a series of switch-on cassettes were developed from the top 4 LMI070-responsive exons found in the RNA-Seq dataset. For this, the minimal intronic intervening sequences necessary to recapitulate splicing of pseudoexons in SF3B3 (FIG. 11E), BENCI (FIG. 11J), C12orf4 (FIG. 11K), and PDXDC2 (FIG. 11L) were cloned upstream of luciferase or eGFP cDNAs (FIGS. 3A and 3C). To limit translation to be only in response to the drug, a Kozak sequence followed by an AUG start codon were positioned within the novel exon to be included in response to LMI070 binding (FIGS. 3A and 3C). HEK293 cells were transfected with the candidate cassettes, treated with LMI070, and luciferase activity or eGFP expression determined 24 h later. Increased luciferase expression was observed for each candidate cassette in response to LMI070, with the SF3B3-on switch showing a more than 100-fold induction (FIG. 3B). Notably, this is 5x the 20-fold induction afforded by the SMN2-on cassette (FIG. 1E). Similarly, eGFP expression was only detected in cells transfected with the SF3B3-on-eGFP cassette in response to LMI070 treatment (FIG. 3D).

While there was measurable baseline luciferase activity in the absence of LMI070 for all candidate cassettes, the SF3B3 cassette had the least background (FIG. 3E). As recent work using ribosome foot printing revealed that non-AUG start codons provide for translation initiation (Ingolia et al., 2009), the presence of in frame non-AUG start codons that could drive luciferase translation in the absence of drug were evaluated. All cassettes contained in frame non-AUG codons, however there was only one in frame non-AUG codon in the SF3B3 cassette (FIG. 3F).

To assess the baseline splicing from the engineered switch cassettes, two different PCR assays were done, one with primers binding the flanking exons to detect all transcripts, and another with a primer binding within the novel spliced exon (FIG. 3G). The LMI070-spliced exon was not detected with primer pairs binding the flanking exons, whereas there was faint signal when priming specifically the novel exon sequence (FIG. 3G). Overall, these results suggest that in the absence of LMI070, the alternative exon may be included in a small fraction of the transcripts, mirroring what was found in the Intropolis dataset (Table 1).

Splicing machinery selection of 5′ and 3′ splice sites pairs is defined by several cis-acting sequences that collectively comprise the ‘splicing code’, including combinations of silencer and enhancer splicing sequences that repress or promote the selection of cryptic or correct splice sites. Using the Human Splicing Finder website (Desmet et al., 2009), the SF3B3 intron sequence was screened for putative silencer and enhancer sequences that could modulate inclusion of the SF3B3 LMI070-spliced pseudoexon (FIG. 3H). Three intronic regions rich in silencing sequences that could repress splicing in the absence of the drug were identified downstream of the pseudoexon. To test their impact on drug-induced control, SF3B3-on-reporter cassettes containing the full intron sequence (SF3B3int; FIG. 11E), intron fragments rich in silencer sequences (SF3B3i1 (FIG. 11F), SF3B3i2 (FIG. 11G), and SF3B3i3 (FIG. 11H)), or an intron fragment less enriched for intronic silencer sequences (SF3B3i4 (FIG. 11I)) were generated (FIG. 3I). HEK293 cells were transfected with the original SF3B3-on switch or the cassettes containing alternate intronic sequences and splicing and luciferase activity determined 24 h later. Whereas cells transfected with SF3B3i4 showed activity similar to the original SF3B3-on construct, luciferase activity was reduced in all the other groups irrespective of drug treatment (FIGS. 3J and 3K). The fold-change in luciferase activity in response to LMI070 was significantly higher in cells transfected with plasmids containing the original intron (>200 fold, FIG. 3K). Surprisingly, the reduction of the luciferase activity was not related to splicing repression of the novel exon, but to the generation of additional spliced transcripts in the absence of the drug (FIGS. 3L and 3M). The original SF3B3-on cassette (hereafter referred to as X^{on}) was used for all further studies.

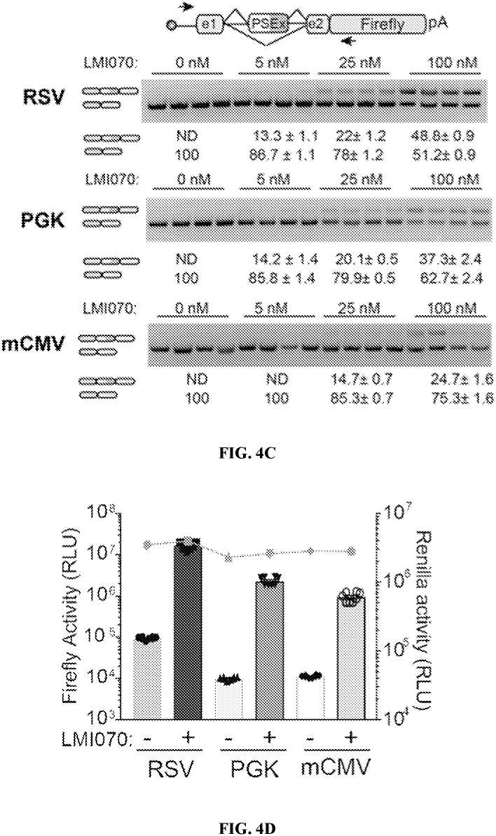

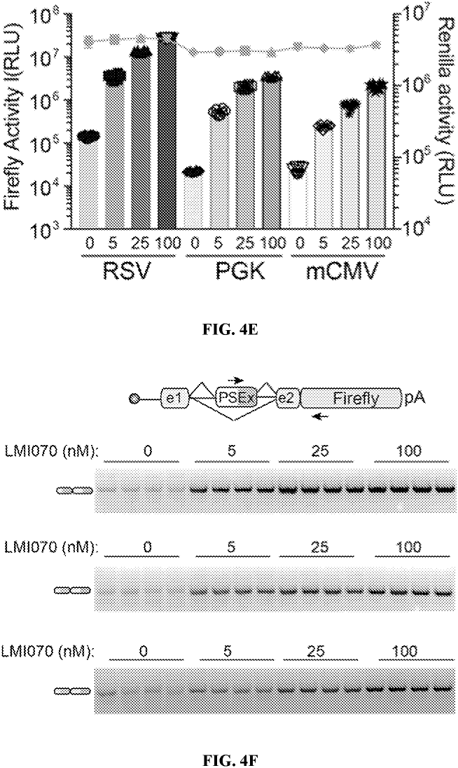

Next, X^{on} was tested for responsiveness when expressed from promoters of varying strengths. HEK293 cells were transfected with expression plasmids containing the X^{on} luciferase encoding cassettes under the control of the Rous sarcoma virus (RSV), the phosphoglycerate kinase (PGK), or the minimal cytomegalovirus (mCMV) promoter. All promoters drove inducible expression (FIG. 4A), with a clear dose response in fold-change luciferase activity (FIGS. 4B, 4D, and 4E) that was mirrored by splicing assessment (FIGS. 4C and 4F). Overall these constructs provide a gradient of induction with RSV>PGK>mCMV.

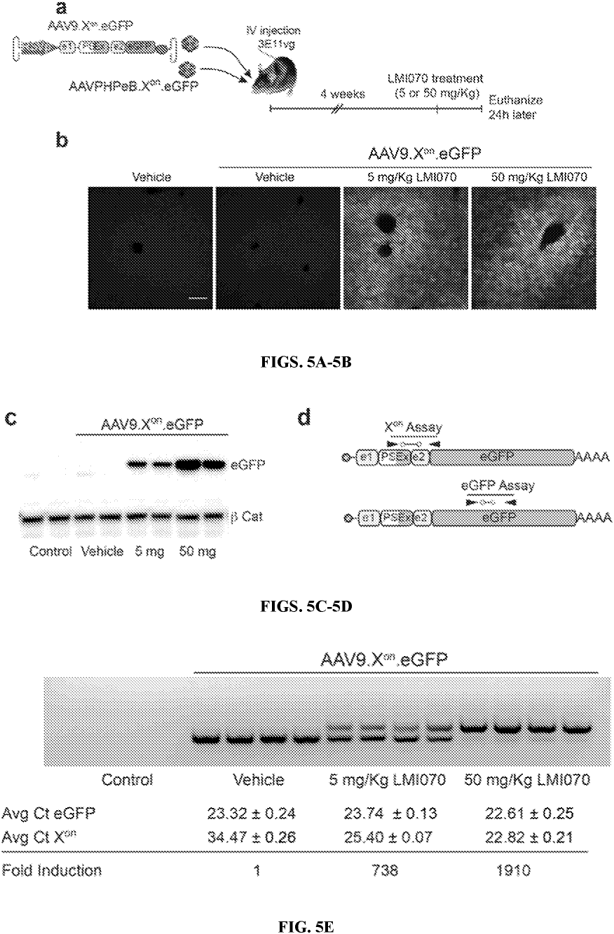

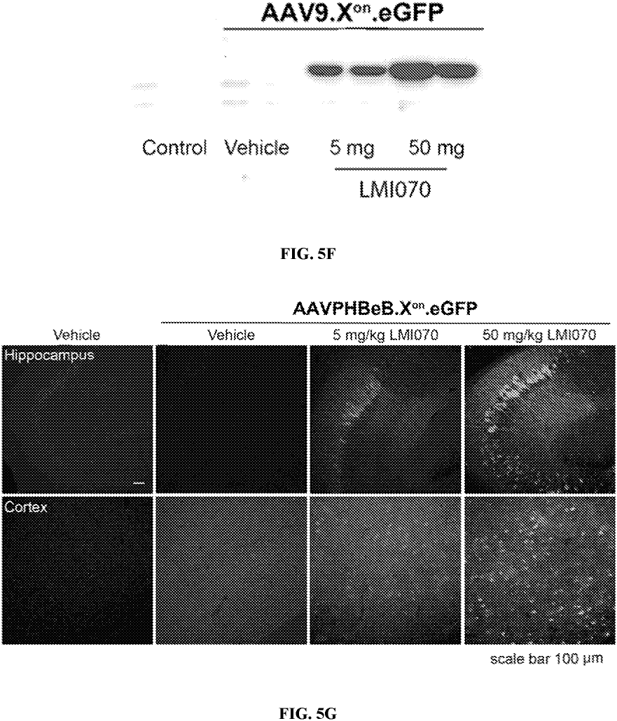

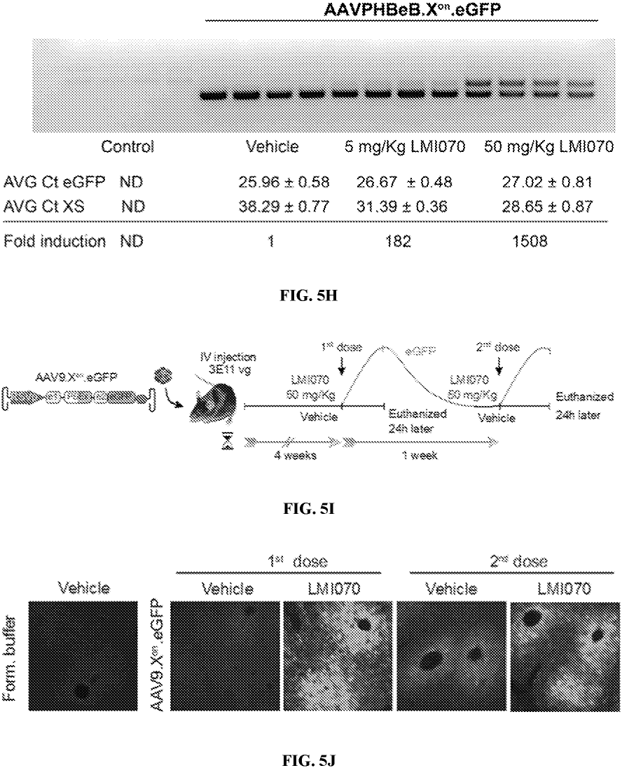

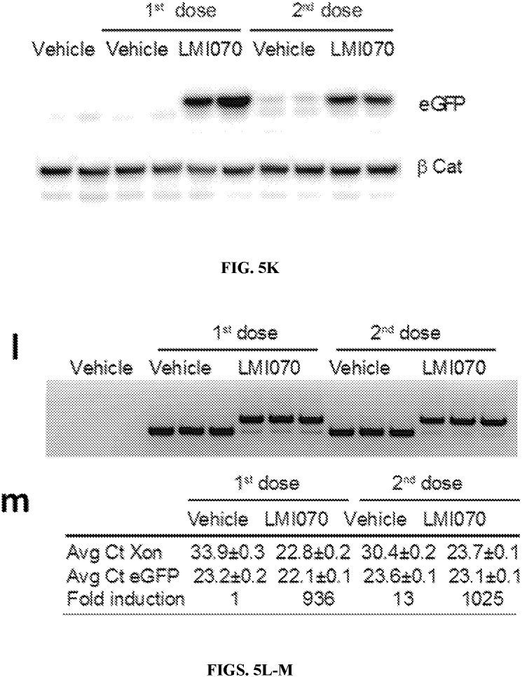

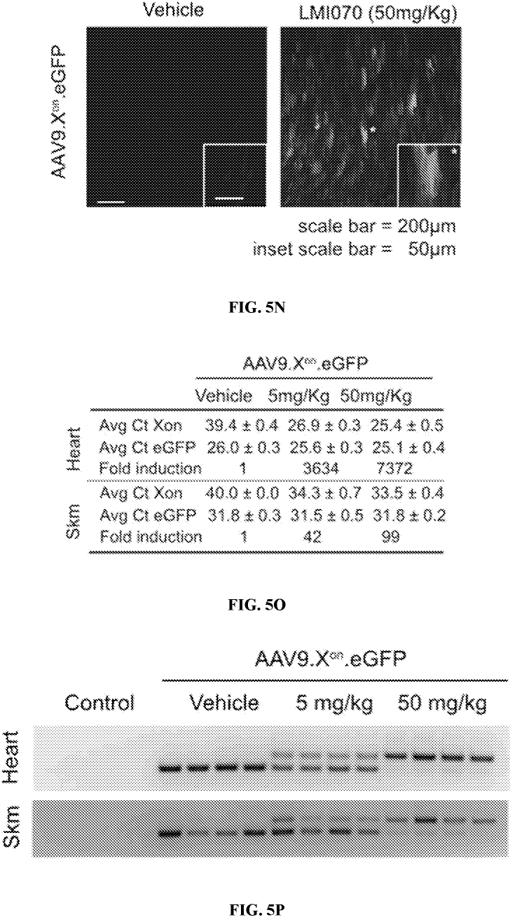

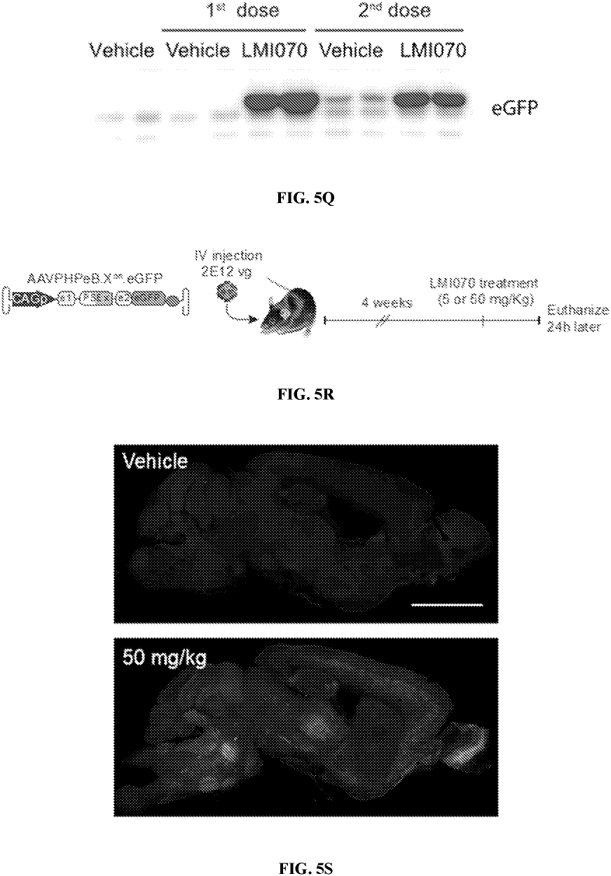

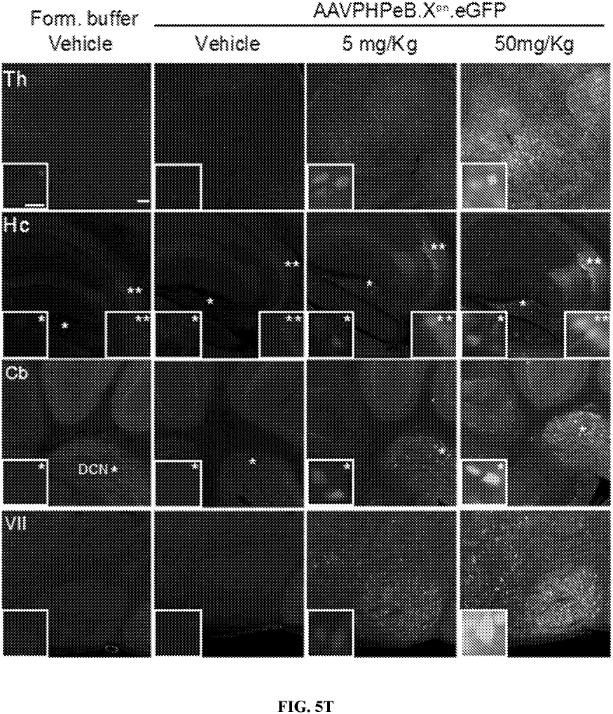

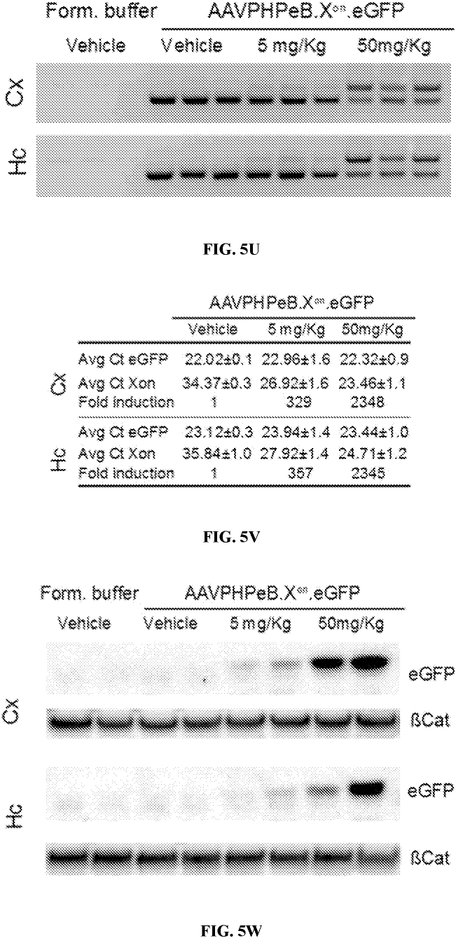

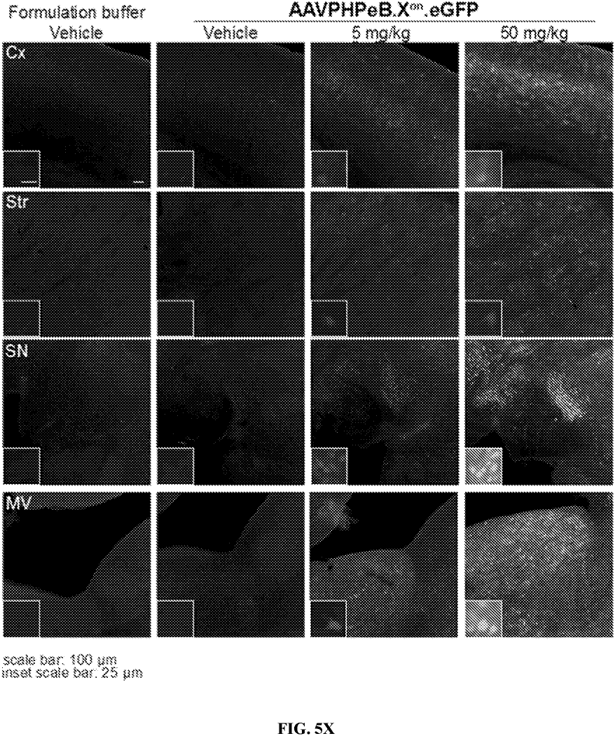

To assess the X^{on} system in vivo, an AAV X^{on} vector was developed and packaged into AAV9 (AAV9.RSV.X^{on}.eGFP). AAV9.X^{on}.eGFP (3E11 vg/mouse) was administrated intravenously (IV) to mice, and 4 weeks later animals were given a single dose of vehicle or LMI070 at 5 or 50 mg/kg and eGFP expression assessed 24 h later (FIG. 5A). There was notable eGFP expression in sections from liver (FIG. 5B). The dose response in eGFP signal noted in tissues by microscopy was confirmed by western blot (FIGS. 5C and 5K) and splicing assay (FIG. 5D). Cumulatively, these data show that in vivo, the X^{on} cassette can be used to drive gene expression from AAV vectors after a single administration of drug. Importantly, protein levels and novel exon splicing directly correlated to the LMI070 dose (FIGS. 5E-5F).

To assess the applicability of this system for brain targeted gene therapies, the X^{on} was packaged into AAVPHPeB (Chan et al., 2017) (AAVPHPeB.X^{on}.eGFP) and delivered IV to mice (3E11 vg/mouse). Again, eGFP expression and novel exon splicing were evident only in response to drug, and in a dose responsive manner (FIGS. 5G and 5H). Importantly, Xon inducibility and gene expression control was also maintained under the expression of stronger promoters (i.e: CAG promoter), as determined by histology, western blot and splicing assay (FIGS. 5R, 5S, 5T, 5U, 5V, 5W, 5X).

Next, Xon was tested for its responsiveness to a second dose of LMI070 as repeat dosing may be required for some gene therapy applications (FIG. 5I). Mice were treated IV with AAV9.X^{on}.eGFP and 4 weeks later, were given one oral dose of either vehicle or LMI070 (50 mg/kg). One day later, some animals were euthanized and induction assessed by histology and splicing assay. The remaining mice underwent one week of drug washout, after which they were given either a second oral dose of LMI070 (50 mg/kg) or vehicle. Induction was evident in liver after the first and the second dose as assessed by histology, western blot, and by splicing assay (FIGS. 5J, 5K, 5L, 5M, 5Q). Induction in skeletal muscle and heart was also notable as assessed by histology (FIG. 5N), and splicing assay (FIGS. 5O&5P), with the relative levels of induction greatest for heart and liver (~3000 and 1000 fold, respectively).

While gene editing approaches provide an enormous opportunity for altering or removing disease alleles, prolonged expression of the editing machinery from viral vectors could be problematic. The editing enzymes are foreign proteins and may induce immune responses, and prolonged gene expression would increase opportunities for off-target editing (Charlesworth et al., 2019; Vakulskas et al., 2018). The utility of the X^{on} system to regulate editing was tested using huntingtin (HTT), a target for gene silencing approaches for Huntington’s disease (HD) (Tabrizi et al., 2019), as an example.

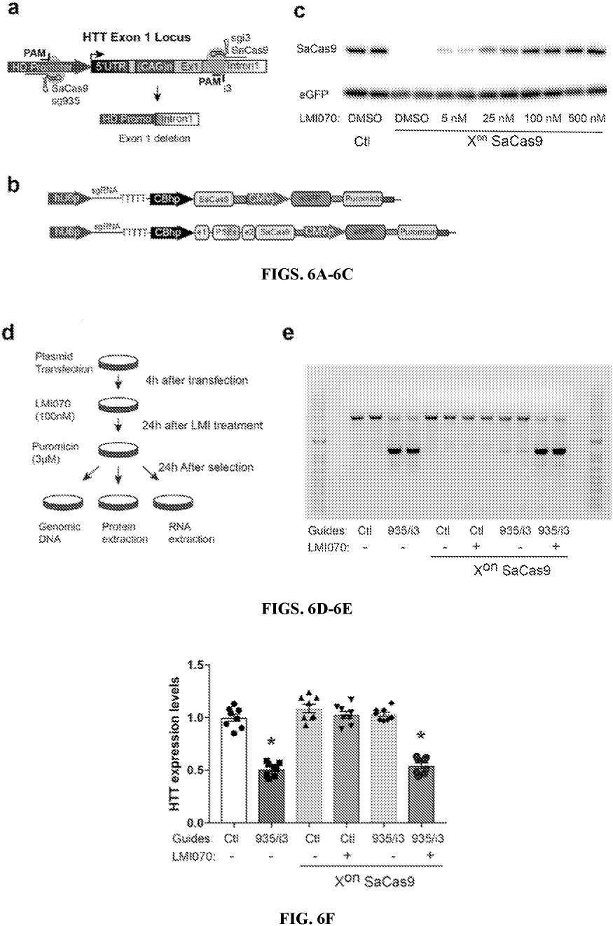

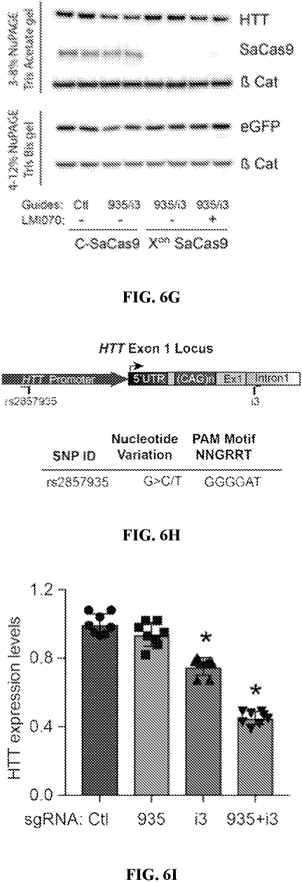

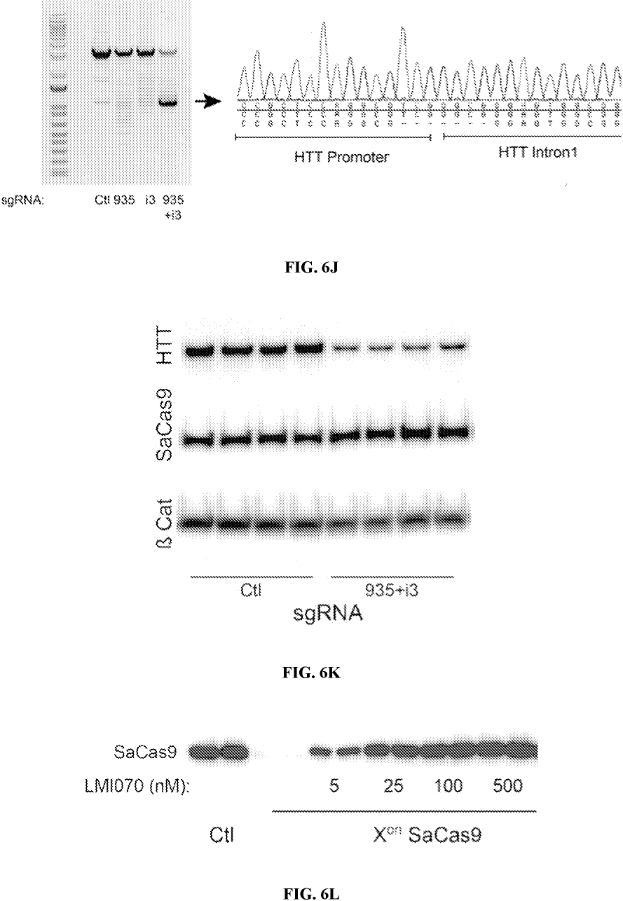

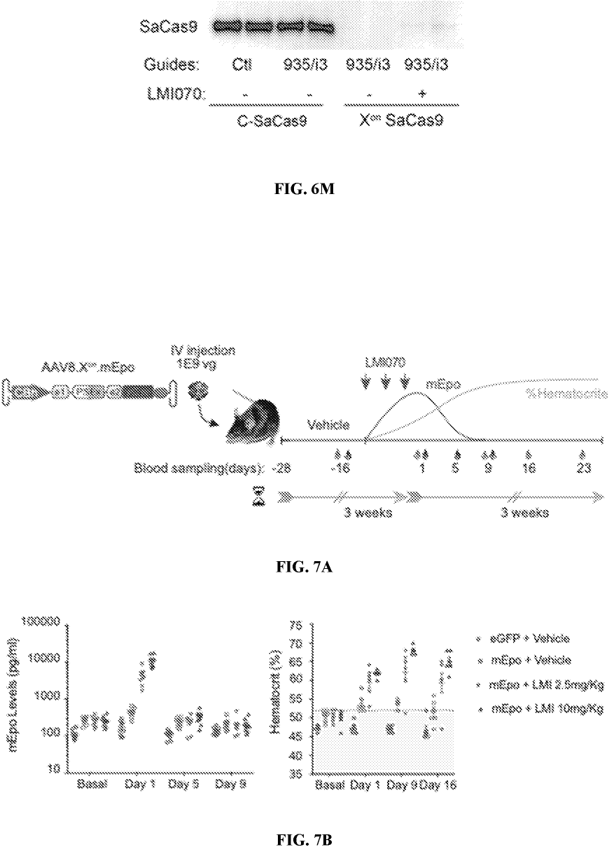

First, gRNAs were designed to mediate SaCas9-mediated deletion of mutant HTT (mHTT) exon 1. For this, a single nucleotide polymorphism (SNP) 5′ to mHTT exon 1 that creates a SaCas9 protospacer adjacent motif was used (PAM; sg935, FIGS. 6A and 6G). When used in combination with a sgRNA targeting the downstream intron (sgi3, FIGS. 6A and 6G) (Monteys et al., 2017), these gRNAs edit HTT exon 1 via SaCas9 and reduce HTT mRNA and protein levels (FIGS. 6H-6K). Next, the X^{on} switch for drug-inducible SaCas9 expression was generated, and compared to the constitutively active cassette (FIG. 6B). There was a clear dose response of SaCas9 expression with LMI070 (FIG. 6C). X^{on}-SaCas9 plus the relevant gRNAs were then transfected in HEK293 cells and the HTT locus, HTT mRNA, and HTT protein levels assessed (FIG. 6D). There was equivalent editing between the samples treated with LMI070 and the samples constitutively expressing SaCas9 (FIG. 6E). More importantly, there was a concomitant reduction at the RNA level upon LMI070 treatment, with HTT transcripts reduced by 50%, similar to the extent noted in cells transfected with the constitutively active editing expression cassette (FIG. 6F). Protein levels were similarly reduced (FIG. 6G). And while minimal editing was detected on cells transfected with active gRNAs and X^{on}-SaCas9 treated with DMSO (FIG. 6E), transcript and protein levels remained unaffected (FIG. 6F). Together, these data show that the X^{on} switch together with allele specific gRNAs directed to mHTT provides an important advance to HD treatment.

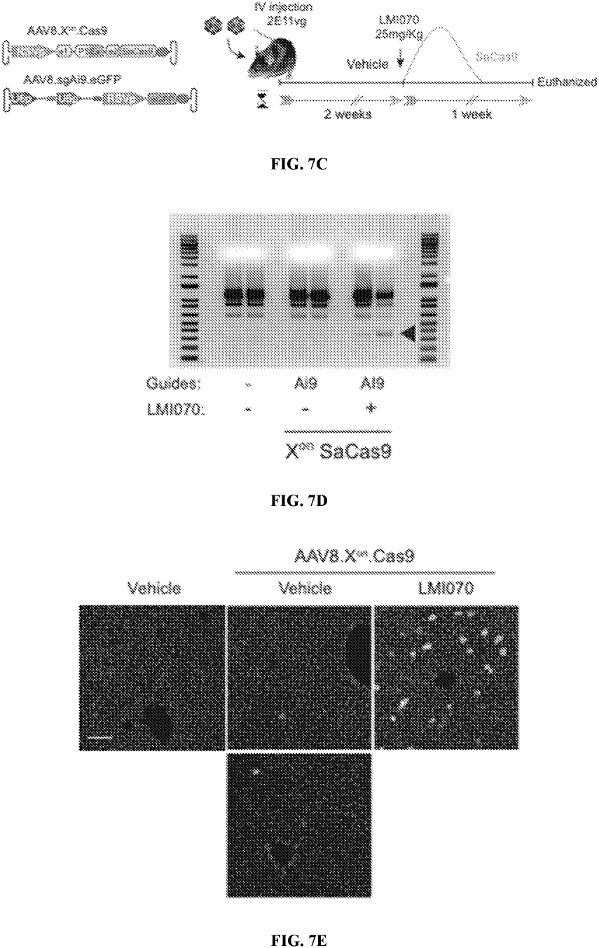

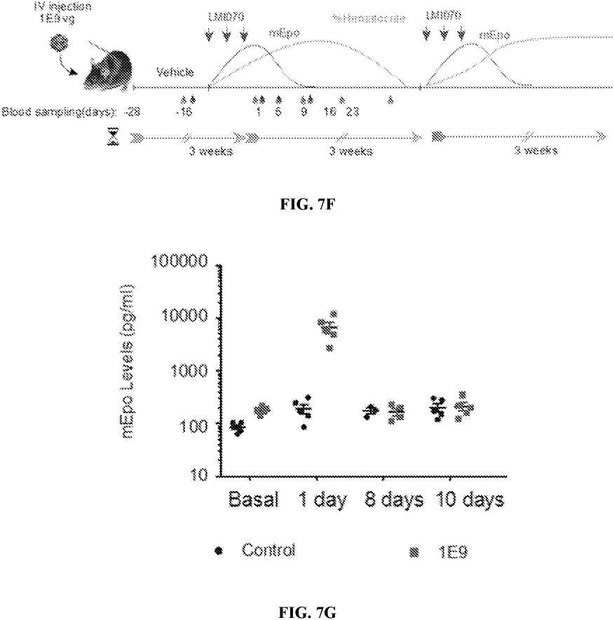

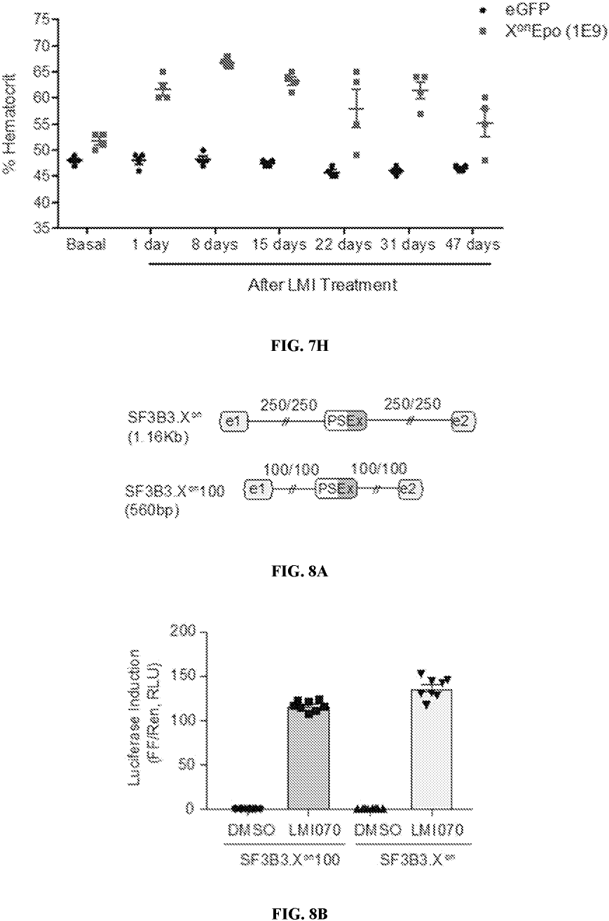

To assess the applicability of Xon for liver targeted therapies, AAV8.Xon.Epo and AAV8.Xon.SaCas9 vectors were generated and delivered to C57Bl6 and Ai14 reporter mice, respectively (FIGS. 7A, 7C, 7F). Induction of Mouse Epo and hematocrit levels were detected only in C57Bl6 mice injected with AAV8.Xon.Epo 24 h after the last LMI070 dose (FIGS. 7B, 7G, 7H). Similarly, editing of the Ai14 reporter genomic locus was only observed on mice injected with AAV8.Xon.SaCas9 virus and treated with LMI070, as determined by PCR to detect editing of the genomic Ai14 locus, and the expression of the tdTomato reporter gene (FIGS. 7D, 7E).

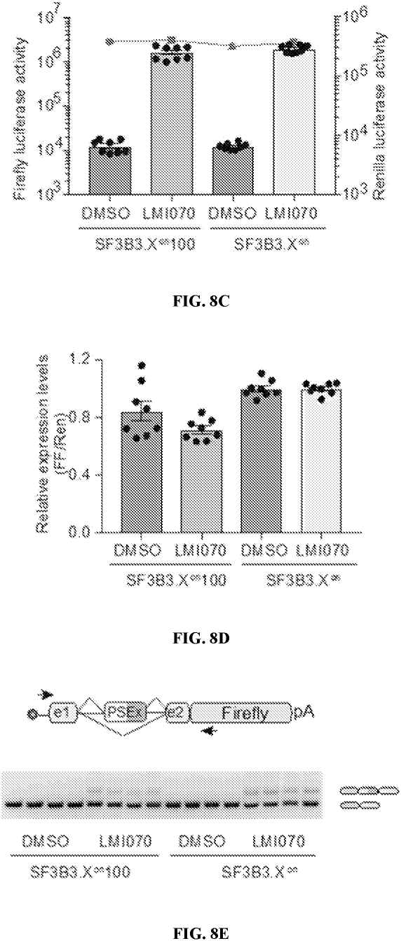

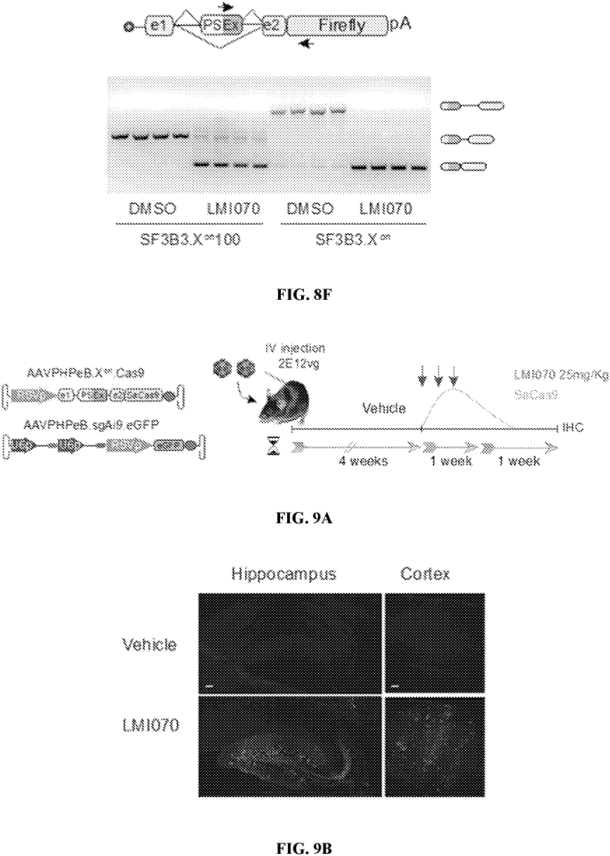

To control the expression of large genes, a compacted version of the SF3B3.Xon cassette was generated with minimized intronic sequences flanking the LMI-induced exon (FIG. 8A). No significant differences were observed between the SF3B3.Xon100 and the SF3B3.Xon cassettes, as determined by luciferase activity and splicing assay (FIGS. 8B, 8C, 8D, 8E, 8F).

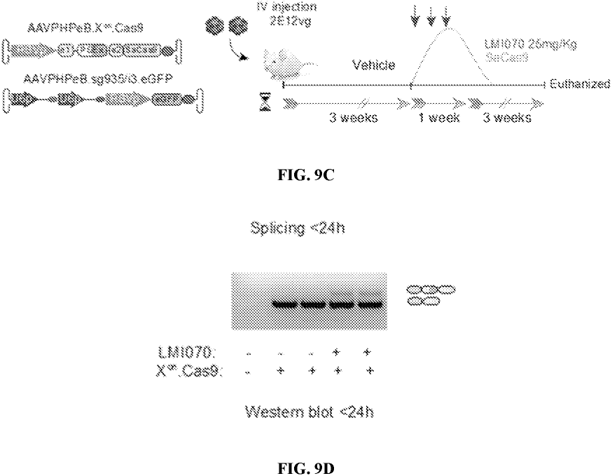

To assess the applicability of Xon for brain targeted therapies, the SF3B3.Xon was packaged into AAVPHPeB vector to control the expression of SaCas9 protein and delivered to transgenic Ai14 reporter mice and BacHD mice (FIGS. 9A, 9C). Editing of the Ai14 reporter genomic locus was only observed on mice treated with LMI070, as demonstrated by the expression of tdTomato protein as result of editing of the Ai14 genomic locus (FIG. 9B).

In summary, a simple, highly adaptable tool for regulated gene expression for in vitro cell biology applications and in vivo evaluation of any protein and testing of new therapies is provided. The utility of the X^{on} system for gene addition and gene editing is shown, and its exquisite control in cells in culture, or in tissues via AAV delivery, is demonstrated. Researchers can use X^{on} in cells or animals to test for additional waves of expression, and use different promoters and drug doses for varying levels of inducibility. The X^{on} tool also gives researchers the means to test gene products that when constitutively expressed are toxic. Indeed, the X^{on} system can be applied to any biological question where fine expression control is desired.

### Example 3 - Regulated Control of Secreted Proteins With a Drug-induced Switch

Most secreted proteins have a signal peptide at their N-terminus that targets the nascent polypeptide to the secretory pathway. Because the AUG start codon in the minigene is positioned within the novel exon (PSEx) that which is included in response to drug, the first several amino acids of the target protein are encoded by a portion of the novel exon (PSEx) and Exon 2 (e2) of the minigene. Thus, for proteins that are secreted, it was considered that this may prevent target proteins from properly entering the secretory pathway by preventing recognition of the signal peptide. As an example, mouse erythropoietin (mEpo) is predicted (99.25%) to have a signal peptide with a cleavage site between amino acids 26 and 27 (92.03%) by SignalP 5.0. With the addition of the minigene sequences that would occur if mEpo were expressed using SF3B3.X^{on}, the prediction of a signal peptide dropped to 57.81% with a predicted cleavage site between amino acids 49 and 50 (53.52%).

As such, modifications of the SF3B3 new exon were made to accommodate secreted proteins. First, the position of the KozacATG was shifted further 3′ within the novel exon (PSEx) so that PSEx only encodes five amino acids, including the initial Met. Second, the sequence of PSEx downstream of the KozacATG was modified to encode the same first five amino acids as mouse Epo. Third, Exon 2 (e2) of the minigene was minimized to encode only one amino acid. Variations of the amino acid encoded by the one codon of the minimized e2 were made, while conserving sequences needed to maintain the splice site. As such, this single codon e2 can encode asparagine (as in SEQ ID NO: 13), arginine (as in SEQ ID NO; 15), or lysine (as in SEQ ID NO: 14). Finally, a BamHI clevage site, which encodes for a glycine and a serine, was kept immediately downstream of the minimized e2.

In order to predict the effect on predicted secretion, the mEpo sequence, starting a codon 7, was inserted downstream of the BamHI site. With an asparagine being encoded by e2, the prediction of a signal peptide increased to 98.32% with a predicted cleavage site between amino acids 28 and 29 (91.08%). With a lysine being encoded by e2, the prediction of a signal peptide increased to 98.83% with a predicted cleavage site between amino acids 28 and 29 (91.58%). With an arginine being encoded by e2, the prediction of a signal peptide increased to 98.89% with a predicted cleavage site between amino acids 28 and 29 (91.58%).

As another example, progranulin is predicted (99.91%) to have a signal peptide with a cleavage site between amino acids 17 and 18 (84.34%) by SignalP 5.0. With the addition of the minigene sequences that would occur if progranulin were expressed using SF3B3.x^{on}, the prediction of a signal peptide dropped to 53.31% with a predicted cleavage site between amino acids 40 and 41 (42.16%). Using the same optimized minigene designs as for mEpo, the progranulin sequence was inserted downstream of the BamHI site and the effect on predicted secretion determined. With an asparagine being encoded by e2, the prediction of a signal peptide increased to 97.90% with a predicted cleavage site between amino acids 24 and 25 (79.54%). With a lysine being encoded by e2, the prediction of a signal peptide increased to 99.25% with a predicted cleavage site between amino acids 24 and 25 (82.27%). With an arginine being encoded by e2, the prediction of a signal peptide increased to 99.45% with a predicted cleavage site between amino acids 24 and 25 (82.57%).

As another example, TPP1 is predicted (98.21%) to have a signal peptide with a cleavage site between amino acids 19 and 20 (63.83%) by SignalP 5.0. With the addition of the minigene sequences that would occur if TPP1 were expressed using SF3B3.X^{on}, the prediction of a signal peptide dropped to 14.27% with no predicted cleavage site. Using the same optimized minigene designs as for mEpo, the TPP1 sequence was inserted downstream of the BamHI site and the effect on predicted secretion determined. With an asparagine being encoded by e2, the prediction of a signal peptide increased to 84.58% with a predicted cleavage site between amino acids 26 and 27 (54.96%). With a lysine being encoded by e2, the prediction of a signal peptide increased to 89.39% with a predicted cleavage site between amino acids 26 and 27 (58.58%). With an arginine being encoded by e2, the prediction of a signal peptide increased to 90.04% with a predicted cleavage site between amino acids 26 and 27 (59.17%).

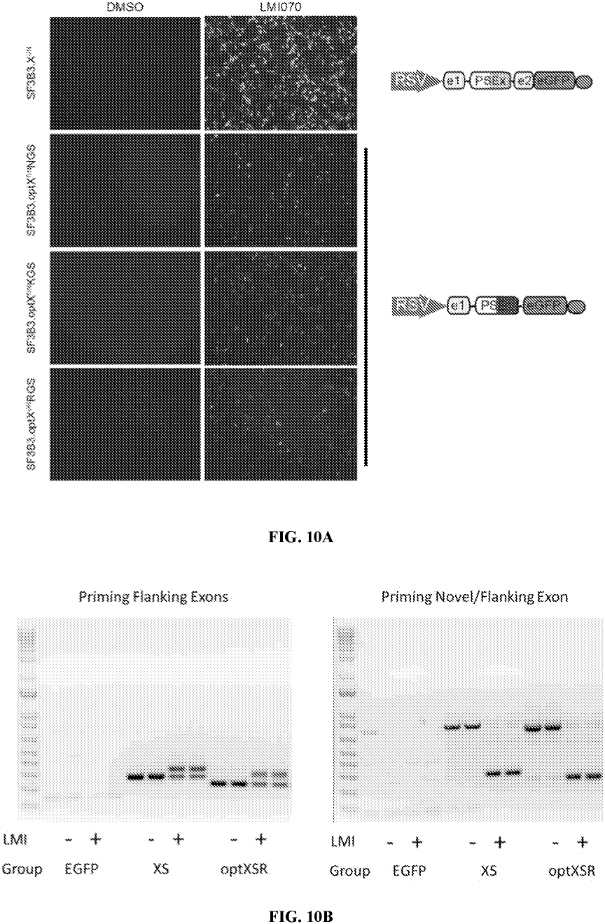

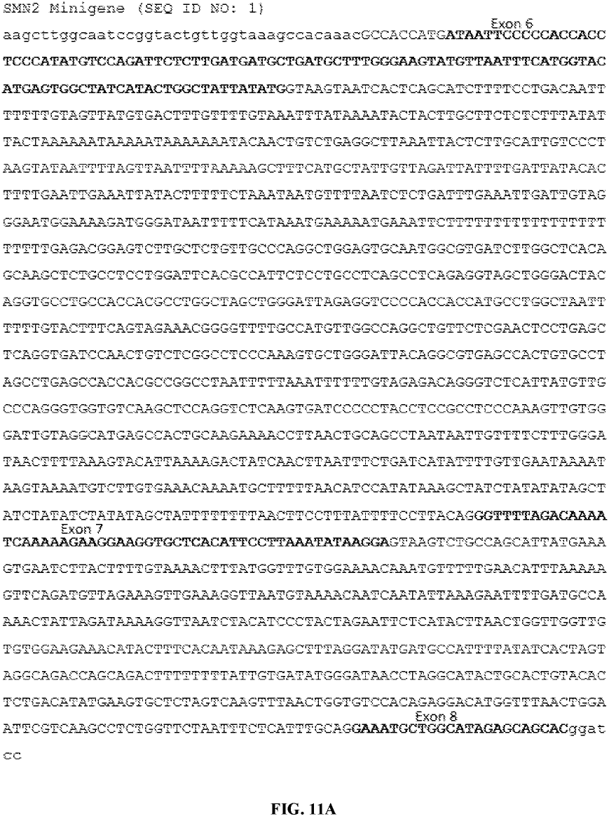

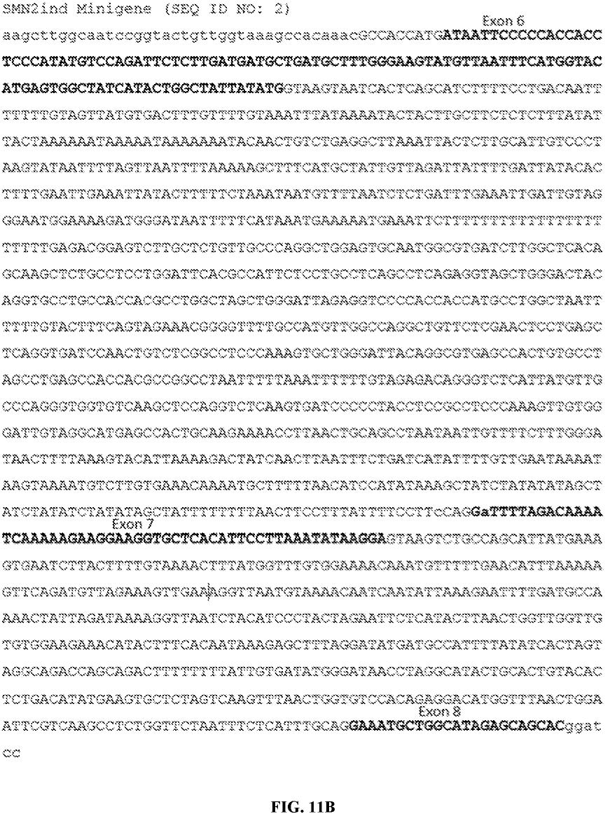

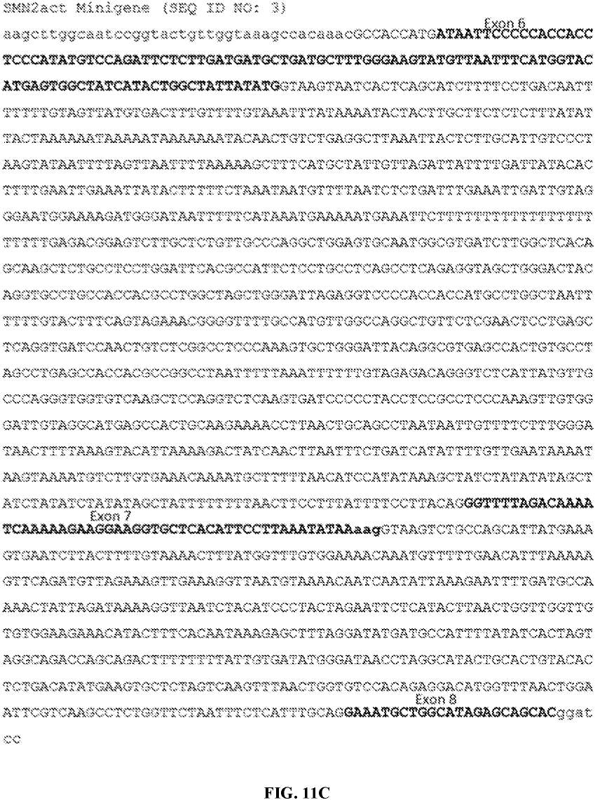

To determine if the optimized minigene designs for secreted protein was able to inducibly express proteins, eGFP was cloned downstream of the BamHI site. As shown in FIG. 10A, the protein expression level from the optimized minigenes was reduced compared to the original, however the inducibility was maintained. The use of a stronger promoter could compensate for the decreased expression level. To assess the baseline splicing from the optimized switch cassettes, two different PCR assays were done, one with primers binding the flanking exons to detect all transcripts, and another with a primer binding within the novel spliced exon (FIG. 10B).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

PCT/US2019/045401, published as WO 2020/033473

Brown, B. D., Venneri, M. A., Zingale, A., Sergi Sergi, L. & Naldini, L. Endogenous microRNA regulation suppresses transgene expression in hematopoietic lineages and enables stable gene transfer. Nat Med 12, 585-591, doi:10.1038/nm1398 (2006).

Cartegni, L., Hastings, M. L., Calarco, J. A., de Stanchina, E. & Krainer, A. R. Determinants of exon 7 splicing in the spinal muscular atrophy genes, SMN1 and SMN2. Am J Hum Genet 78, 63-77, doi:10.1086/498853 (2006).

Cartegni, L. & Krainer, A. R. Disruption of an SF2/ASF-dependent exonic splicing enhancer in SMN2 causes spinal muscular atrophy in the absence of SMN1. Nat Genet 30, 377-384, doi:10.1038/ng854 (2002).

Chan, K. Y. et al. Engineered AAVs for efficient noninvasive gene delivery to the central and peripheral nervous systems. Nat Neurosci 20, 1172-1179, doi:10.1038/nn.4593 (2017).

Charlesworth, C. T. et al. Identification of preexisting adaptive immunity to Cas9 proteins in humans. Nat Med 25, 249-254, doi:10.1038/s41591-018-0326-x (2019).

Cheung, A. K. et al. Discovery of Small Molecule Splicing Modulators of Survival Motor Neuron-2 (SMN2) for the Treatment of Spinal Muscular Atrophy (SMA). J Med Chem 61, 11021-11036, doi:10.1021/acs.jmedchem.8b01291 (2018).

Desmet, F. O. et al. Human Splicing Finder: an online bioinformatics tool to predict splicing signals. Nucleic Acids Res 37, e67, doi:10.1093/nar/gkp215 (2009).

Dobin, A. & Gingeras, T. R. Mapping RNA-seq Reads with STAR. Curr Protoc Bioinformatics 51, 11 14 11-11 14 19, doi:10.1002/0471250953.bi1114s51 (2015).

Domenger, C. & Grimm, D. Next-generation AAV vectors-do not judge a virus (only) by its cover. Hum Mol Genet 28, R3-R14, doi:10.1093/hmg/ddz148 (2019).

Ingolia, N. T., Ghaemmaghami, S., Newman, J. R. & Weissman, J. S. Genome-wide analysis in vivo of translation with nucleotide resolution using ribosome profiling. Science 324, 218-223, doi:10.1126/science.1168978 (2009).

Katz, Y. et al. Quantitative visualization of alternative exon expression from RNA-seq data. Bioinformatics 31, 2400-2402, doi:10.1093/bioinformatics/btv034 (2015).

Kent, W. J. et al. The human genome browser at UCSC. Genome Res 12, 996-1006, doi:10.1101/gr.229102 (2002).

Love, M. I., Huber, W. & Anders, S. Moderated estimation of fold change and dispersion for RNA-seq data with DESeq2. Genome Biol 15, 550, doi:10.1186/s13059-014-0550-8 (2014).

Monteys, A. M., Ebanks, S. A., Keiser, M. S. & Davidson, B. L. CRISPR/Cas9 Editing of the Mutant Huntingtin Allele In Vitro and In Vivo. Mol Ther 25, 12-23, doi:10.1016/j.ymthe.2016.11.010 (2017).

Nellore, A. et al. Human splicing diversity and the extent of unannotated splice junctions across human RNA-seq samples on the Sequence Read Archive. Genome Biol 17, 266, doi:10.1186/s13059-016-1118-6 (2016).

Palacino, J. et al. SMN2 splice modulators enhance U1-pre-mRNA association and rescue SMA mice. Nat Chem Biol 11, 511-517, doi:10.1038/nchembio.1837 (2015).

Ratni, H. et al. Specific Correction of Alternative Survival Motor Neuron 2 Splicing by Small Molecules: Discovery of a Potential Novel Medicine To Treat Spinal Muscular Atrophy. J Med Chem 59, 6086-6100, doi:10.1021/acs.jmedchem.6b00459 (2016).

Tabrizi, S. J., Ghosh, R. & Leavitt, B. R. Huntingtin Lowering Strategies for Disease Modification in Huntington’s Disease. Neuron 101, 801-819, doi:10.1016/j.neuron.2019.01.039 (2019).

Thorvaldsdottir, H., Robinson, J. T. & Mesirov, J. P. Integrative Genomics Viewer (IGV): high-performance genomics data visualization and exploration. Brief Bioinform 14, 178-192, doi:10.1093/bib/bbs017 (2013).

Vakulskas, C. A. et al. A high-fidelity Cas9 mutant delivered as a ribonucleoprotein complex enables efficient gene editing in human hematopoietic stem and progenitor cells. Nat Med 24, 1216-1224, doi:10.1038/s41591-018-0137-0 (2018).

Wang, J., Schultz, P. G. & Johnson, K. A. Mechanistic studies of a small-molecule modulator of SMN2 splicing. Proc Natl Acad Sci U S A 115, E4604-E4612, doi:10.1073/pnas.1800260115 (2018).

What is claimed is:

1. A nucleic acid molecule comprising a first expression cassette comprising, from 5′ to 3′, (a) a minigene having an alternatively spliced exon and (b) an encoded gene, wherein the mini gene comprises, from 5′ to 3′, Exon 1, Intron 1, Exon 2, Intron 2, and Exon 3, wherein Exon 2 is the alternatively spliced exon, and wherein Exon 2 comprises translation initiation regulatory sequences.

2. The nucleic acid molecule of claim 1, wherein the alternatively spliced exon is a pseudoexon.

3-4. (canceled)

5. The nucleic acid molecule of claim 1, wherein the number of nucleotides present in Exon 2 is not divisible by 3.

6. The nucleic acid molecule of claim 1, wherein Exon 3 comprises a stop codon that is in frame when Exon 2 is skipped.

7. The nucleic acid molecule of claim 1, wherein the encoded gene is in frame with the translation initiation regulatory sequence in Exon 2.

8. The nucleic acid molecule of claim 1, wherein the encoded gene encodes a signal peptide, wherein the amino acids encoded by Exon 2 correspond to a sequence of a predicted signal peptide.

9-11. (canceled)

12. The nucleic acid molecule of claim 1, wherein Exon 2 comprises a sequence having at least 90% identity to nucleotides 1203-1257 of SEQ ID NO: 1, and optionally wherein Intron 1 comprises a sequence having at least 90% identity to nucleotides 159-1202 of SEQ ID NO: 1 and/or Intron 2 comprises a sequence having at least 90% identity to nucleotides 1258-1701 of SEQ ID NO: 1.

13-15. (canceled)

16. The nucleic acid molecule of claim 1, wherein Exon 2 comprises a sequence having at least 90% identity to nucleotides 595-653 of SEQ ID NO: 4, optionally wherein Intron 1 comprises a sequence having at least 90% identity to nucleotides 97-594 of SEQ ID NO: 4 and/or Intron 2 comprises a sequence having at least 90% identity to nucleotides 654-1153 of SEQ ID NO: 4.

17-19. (canceled)

20. The nucleic acid molecule of claim 1, wherein Exon 2 comprises a sequence having at least 90% identity to nucleotides 427-471 of SEQ ID NO: 10, optionally wherein Intron 1 comprises a sequence having at least 90% identity to nucleotides 103-426 of SEQ ID NO: 10 and/or Intron 2 comprises a sequence having at least 90% identity to nucleotides 472-834 of SEQ ID NO: 10.

21-23. (canceled)

24. The nucleic acid molecule of claim 1, wherein Exon 2 comprises a sequence having at least 90% identity to nucleotides 621-759 of SEQ ID NO: 11, optionally wherein Intron 1 comprises a sequence having at least 90% identity to nucleotides 119-620 of SEQ ID NO: 11 and/or Intron 2 comprises a sequence having at least 90% identity to nucleotides 760-1228 of SEQ ID NO: 11.

25-27. (canceled)

28. The nucleic acid molecule of claim 1, wherein Exon 2 comprises a sequence having at least 90% identity to nucleotides 750-817 of SEQ ID NO: 12,optionally wherein Intron 1 comprises a sequence having at least 90% identity to nucleotides 99-749 of SEQ ID NO: 12 and/or wherein Intron 2 comprises a sequence having at least 90% identity to nucleotides 818-936 of SEQ ID NO: 12.

29-31. (canceled)

32. The nucleic acid molecule of claim 1, wherein Exon 2 comprises a sequence having at least 90% identity to nucleotides 593-650 of SEQ ID NO: 13, optionally wherein Exon 3 comprises a sequence having at least 90% identity to nucleotides 1149-1153 of SEQ ID NO: 13, 14, or 15 and/or wherein Intron 1 comprises a sequence having at least 90% identity to nucleotides 96-592 of SEQ ID NO: 13 and/or wherein Intron 2 comprises a sequence having at least 90% identity to nucleotides 651-1148 of SEQ ID NO: 13.

33-37. (canceled)

38. The nucleic acid molecule of claim 1, wherein the minigene comprises a sequence according to any of SEQ ID NOs: 1, 4, and 10-15.

39. The nucleic acid molecule of claim 1, wherein the minigene comprises fewer than 2000, fewer than 1900, fewer than 1800, fewer than 1700, fewer than 1600, fewer than 1500, fewer than 1400, fewer than 1300, fewer than 1200, fewer than 1100, fewer than 1000, fewer than 900, fewer than 800, fewer than 700, fewer than 600 or fewer than 500 nucleotides.

40. (canceled)

41. The nucleic acid molecule of claim 1, wherein the encoded gene encodes an inhibitory RNA, a therapeutic protein, a Cas9 protein, or a transactivator protein.

42-44. (canceled)

45. The nucleic acid molecule of claim 1, wherein the minigene and the encoded gene are separated by a cleavable peptide.

46. The nucleic acid molecule of claim 1, wherein the first expression cassette is operably linked to a first promoter.

47-48. (canceled)

49. The nucleic acid molecule of claim 1, further comprising a second expression cassette.

50. The nucleic acid molecule of claim 49, wherein the second expression cassette comprises a nucleic acid sequence encoding a guide RNA operably linked to a second promoter.

51. The nucleic acid molecule of claim 49, wherein the second expression cassette comprises a nucleic acid sequence encoding a therapeutic protein, an inhibitory RNA, or a Cas9 protein, wherein the nucleic acid sequence is operably linked to a second promoter, wherein the second promoter is activated by the transactivator encoded by the first expression cassette.

52. A cell comprising the nucleic acid molecule of claim 1.

53. A recombinant adeno-associated virus (rAAV) vector comprising an AAV capsid protein and nucleic acid molecule of claim 1.

54. A method of inducing the expression of the encoded gene in a cell of claim 52, the method comprising contacting the cell with a splicing modifier drug.

55-56. (canceled)

57. A method of administering the encoded gene to a patient in need thereof, the method comprising administering the nucleic acid molecule of claim 1 to the patient.

58-90. (canceled)


SEQUENCE LISTING (31 sequences)

SEQ ID NO: 1 - DNA, 1730 bp, Artificial sequence
  SMN2 Minigene
aagcttggcaatccggtactgttggtaaagccacaaacgccaccatgataattcccccaccacctcccatatgtccagattctcttgatgatgctgatgctttgggaagtatgttaatttcatggtacatgagtggctatcatactggctattatatggtaagtaatcactcagcatcttttcctgacaatttttttgtagttatgtgactttgttttgtaaatttataaaatactacttgcttctctctttatattactaaaaaataaaaataaaaaaatacaactgtctgaggcttaaattactcttgcattgtccctaagtataattttagttaattttaaaaagctttcatgctattgttagattattttgattatacacttttgaattgaaattatactttttctaaataatgttttaatctctgatttgaaattgattgtagggaatggaaaagatgggataatttttcataaatgaaaaatgaaattcttttttttttttttttttttttgagacggagtcttgctctgttgcccaggctggagtgcaatggcgtgatcttggctcacagcaagctctgcctcctggattcacgccattctcctgcctcagcctcagaggtagctgggactacaggtgcctgccaccacgcctggctagctgggattagaggtccccaccaccatgcctggctaattttttgtactttcagtagaaacggggttttgccatgttggccaggctgttctcgaactcctgagctcaggtgatccaactgtctcggcctcccaaagtgctgggattacaggcgtgagccactgtgcctagcctgagccaccacgccggcctaatttttaaattttttgtagagacagggtctcattatgttgcccagggtggtgtcaagctccaggtctcaagtgatccccctacctccgcctcccaaagttgtgggattgtaggcatgagccactgcaagaaaaccttaactgcagcctaataattgttttctttgggataacttttaaagtacattaaaagactatcaacttaatttctgatcatattttgttgaataaaataagtaaaatgtcttgtgaaacaaaatgctttttaacatccatataaagctatctatatatagctatctatatctatatagctattttttttaacttcctttattttccttacagggttttagacaaaatcaaaaagaaggaaggtgctcacattccttaaatataaggagtaagtctgccagcattatgaaagtgaatcttacttttgtaaaactttatggtttgtggaaaacaaatgtttttgaacatttaaaaagttcagatgttagaaagttgaaaggttaatgtaaaacaatcaatattaaagaattttgatgccaaaactattagataaaaggttaatctacatccctactagaattctcatacttaactggttggttgtgtggaagaaacatactttcacaataaagagctttaggatatgatgccattttatatcactagtaggcagaccagcagacttttttttattgtgatatgggataacctaggcatactgcactgtacactctgacatatgaagtgctctagtcaagtttaactggtgtccacagaggacatggtttaactggaattcgtcaagcctctggttctaatttctcatttgcaggaaatgctggcatagagcagcacggatcc

SEQ ID NO: 2 - DNA, 1730 bp, Artificial sequence
  SMN2ind Minigene
aagcttggcaatccggtactgttggtaaagccacaaacgccaccatgataattcccccaccacctcccatatgtccagattctcttgatgatgctgatgctttgggaagtatgttaatttcatggtacatgagtggctatcatactggctattatatggtaagtaatcactcagcatcttttcctgacaatttttttgtagttatgtgactttgttttgtaaatttataaaatactacttgcttctctctttatattactaaaaaataaaaataaaaaaatacaactgtctgaggcttaaattactcttgcattgtccctaagtataattttagttaattttaaaaagctttcatgctattgttagattattttgattatacacttttgaattgaaattatactttttctaaataatgttttaatctctgatttgaaattgattgtagggaatggaaaagatgggataatttttcataaatgaaaaatgaaattcttttttttttttttttttttttgagacggagtcttgctctgttgcccaggctggagtgcaatggcgtgatcttggctcacagcaagctctgcctcctggattcacgccattctcctgcctcagcctcagaggtagctgggactacaggtgcctgccaccacgcctggctagctgggattagaggtccccaccaccatgcctggctaattttttgtactttcagtagaaacggggttttgccatgttggccaggctgttctcgaactcctgagctcaggtgatccaactgtctcggcctcccaaagtgctgggattacaggcgtgagccactgtgcctagcctgagccaccacgccggcctaatttttaaattttttgtagagacagggtctcattatgttgcccagggtggtgtcaagctccaggtctcaagtgatccccctacctccgcctcccaaagttgtgggattgtaggcatgagccactgcaagaaaaccttaactgcagcctaataattgttttctttgggataacttttaaagtacattaaaagactatcaacttaatttctgatcatattttgttgaataaaataagtaaaatgtcttgtgaaacaaaatgctttttaacatccatataaagctatctatatatagctatctatatctatatagctattttttttaacttcctttattttccttccaggattttagacaaaatcaaaaagaaggaaggtgctcacattccttaaatataaggagtaagtctgccagcattatgaaagtgaatcttacttttgtaaaactttatggtttgtggaaaacaaatgtttttgaacatttaaaaagttcagatgttagaaagttgaaaggttaatgtaaaacaatcaatattaaagaattttgatgccaaaactattagataaaaggttaatctacatccctactagaattctcatacttaactggttggttgtgtggaagaaacatactttcacaataaagagctttaggatatgatgccattttatatcactagtaggcagaccagcagacttttttttattgtgatatgggataacctaggcatactgcactgtacactctgacatatgaagtgctctagtcaagtttaactggtgtccacagaggacatggtttaactggaattcgtcaagcctctggttctaatttctcatttgcaggaaatgctggcatagagcagcacggatcc

SEQ ID NO: 3 - DNA, 1730 bp, Artificial sequence
  SMN2act Minigene
aagcttggcaatccggtactgttggtaaagccacaaacgccaccatgataattcccccaccacctcccatatgtccagattctcttgatgatgctgatgctttgggaagtatgttaatttcatggtacatgagtggctatcatactggctattatatggtaagtaatcactcagcatcttttcctgacaatttttttgtagttatgtgactttgttttgtaaatttataaaatactacttgcttctctctttatattactaaaaaataaaaataaaaaaatacaactgtctgaggcttaaattactcttgcattgtccctaagtataattttagttaattttaaaaagctttcatgctattgttagattattttgattatacacttttgaattgaaattatactttttctaaataatgttttaatctctgatttgaaattgattgtagggaatggaaaagatgggataatttttcataaatgaaaaatgaaattcttttttttttttttttttttttgagacggagtcttgctctgttgcccaggctggagtgcaatggcgtgatcttggctcacagcaagctctgcctcctggattcacgccattctcctgcctcagcctcagaggtagctgggactacaggtgcctgccaccacgcctggctagctgggattagaggtccccaccaccatgcctggctaattttttgtactttcagtagaaacggggttttgccatgttggccaggctgttctcgaactcctgagctcaggtgatccaactgtctcggcctcccaaagtgctgggattacaggcgtgagccactgtgcctagcctgagccaccacgccggcctaatttttaaattttttgtagagacagggtctcattatgttgcccagggtggtgtcaagctccaggtctcaagtgatccccctacctccgcctcccaaagttgtgggattgtaggcatgagccactgcaagaaaaccttaactgcagcctaataattgttttctttgggataacttttaaagtacattaaaagactatcaacttaatttctgatcatattttgttgaataaaataagtaaaatgtcttgtgaaacaaaatgctttttaacatccatataaagctatctatatatagctatctatatctatatagctattttttttaacttcctttattttccttacagggttttagacaaaatcaaaaagaaggaaggtgctcacattccttaaatataaaaggtaagtctgccagcattatgaaagtgaatcttacttttgtaaaactttatggtttgtggaaaacaaatgtttttgaacatttaaaaagttcagatgttagaaagttgaaaggttaatgtaaaacaatcaatattaaagaattttgatgccaaaactattagataaaaggttaatctacatccctactagaattctcatacttaactggttggttgtgtggaagaaacatactttcacaataaagagctttaggatatgatgccattttatatcactagtaggcagaccagcagacttttttttattgtgatatgggataacctaggcatactgcactgtacactctgacatatgaagtgctctagtcaagtttaactggtgtccacagaggacatggtttaactggaattcgtcaagcctctggttctaatttctcatttgcaggaaatgctggcatagagcagcacggatcc

SEQ ID NO: 4 - DNA, 1199 bp, Artificial sequence
  SF3B3 Minigene
aagcttggcaatccggtactgttggtaaatttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgtggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttgaattgtgtgaaatttcaaatattggattaggaaatacaaagttactgaaagtgaggtactaatgtttataaaataaaaactttttcttgccatttgcagatttaacatttttgagtcaatccaagtgccaccatgcaggaggttcatgattgtgtagagtaagacataattttgttgaggtttaactctgaatacttaatgtggtactgaatacttaatgtggtactgagaggcagcctaactgaccacacagcattcatatttcatgttgttattttctctgatcctcattatggctcttcctgcgggtttgagtcttcatttgggtaatttagttattctttgctgcatttgattaaggaaatacgtatatgggaatttggagatttgtgactgacttcactatgtctaatttttttttgagacggagttttgctctgttgcccaggccggagtgcagtggcacagtcttggctcacagcaacctctacctcctgggttcaagcgattctcctgccttagcctcccgagtagctgggattacaggcatgtgccaccacgcctggctaatttttgtatttttagtagagacggggtttcaccatggtggccaggctgggttctggttgtttatgatctttattttttggtgatctaggaaccaaacaacaagaaattgttgtttcccgtgggtaagaggatcc

SEQ ID NO: 5 - DNA, 2142 bp, Artificial sequence
  SF3B3int Minigene
aagcttggcaatccggtactgttggtaaatttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgtggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttgaattgtgtgaaatttcaaatattggattaggaaatacaaagttactgaaagtgaggtactaatgtttataaaataaaaactttttcttgccatttgcagatttaacatttttgagtcaatccaagtgccaccatgcaggaggttcatgattgtgtagagtaagacataattttgttgaggtttaactctgaatacttaatgtggtactgaatacttaatgtggtactgagaggcagcctaactgaccacacagcattcatatttcatgttgttattttctctgatcctcattatggctcttcctgcgggtttgagtcttcatttgggtaatttagttattctttgctgcatttgattaaggaaatacgtatatgggaatttggagatttgtgactgacttcactatgtaatgcaatgaatctcagctggagtttggatagaatttttgaaagcacgtttttgttttttgtttgttttgtttttgttttttgtttgttttgtttttgtttttgttttttccttgaaacagagtcctgctctgtcacccaggctggagcgcagtggtgtgatcttggctcactgccacctccgcctcctgggttcaagcgattctcctgcctcagcctcctgagtagctgggattacatgcgtgcaccaccatgcccggctaatttttcatatttttagtagagatgaggtttcaccatattggccaggctgatctcgaattcctgacctcgtgatccacccaccttagcctcccaaagtgctgggattacaggcatgagctactgtgccctgctgaaagcacatttttaatactaattttatctttcaaattccttttccaattcagtcttcctttttatctaaaatgatggagaagttttcttttttttttttttttttttgagacgtagtcttgctgtgttgcccaggctggagtacagtagtgcgacctcggctcactgcaaactccgtctcccaggttcaagcgattctcctgtgtcagcctctggagtagctgggattataggcacgtgccaccacacctggctaatttttgtatttttagtagagatgggatcccaccatgttggccaggctggtttcaaactcctgacctcaagtgatctgcctgcctcagcctcccaaagtgctgggataacaggtgtgagccactgtgcgtggccttttttttttttttttttttttttagagacgatatcttgctgcgtttcccaggatggagtgcaatggcaggattataacccactacattctctgaactcctgggttcaaactgtcctcccacgtagctgggactagaggcacaggccatcacacctgactaatttttttttgagacggagttttgctctgttgcccaggccggagtgcagtggcacagtcttggctcacagcaacctctacctcctgggttcaagcgattctcctgccttagcctcccgagtagctgggattacaggcatgtgccaccacgcctggctaatttttgtatttttagtagagacggggtttcaccatggtggccaggctgggttctggttgtttatgatctttattttttggtgatctaggaaccaaacaacaagaaattgttgtttcccgtgggtaagaggatcc

SEQ ID NO: 6 - DNA, 1642 bp, Artificial sequence
  SF3B3int1 Minigene
aagcttggcaatccggtactgttggtaaatttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgtggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttgaattgtgtgaaatttcaaatattggattaggaaatacaaagttactgaaagtgaggtactaatgtttataaaataaaaactttttcttgccatttgcagatttaacatttttgagtcaatccaagtgccaccatgcaggaggttcatgattgtgtagagtaagacataattttgttgaggtttaactctgaatacttaatgtggtactgaatacttaatgtggtactgagaggcagcctaactgaccacacagcattcatatttcatgttgttattttctctgatcctcattatggctcttcctgcgggtttgagtcttcatttgggtaatttagttattctttgctgcatttgattaaggaaatacgtatatgggaatttggagatttgtgactgacttcactatgtaatgcaatgaatctcagctggagtttggatagaatttttgaaagcacgtttttgttttttgtttgttttgtttttgttttttgtttgttttgtttttgtttttgttttttccttgaaacagagtcctgctctgtcacccaggctggagcgcagtggtgtgatcttggctcactgccacctccgcctcctgggttcaagcgattctcctgcctcagcctcctgagtagctgggattacatgcgtgcaccaccatgcccggctaatttttcatatttttagtagagatgaggtttcaccatattggccaggctgatctcgatcttgctgcgtttcccaggatggagtgcaatggcaggattataacccactacattctctgaactcctgggttcaaactgtcctcccacgtagctgggactagaggcacaggccatcacacctgactaatttttttttgagacggagttttgctctgttgcccaggccggagtgcagtggcacagtcttggctcacagcaacctctacctcctgggttcaagcgattctcctgccttagcctcccgagtagctgggattacaggcatgtgccaccacgcctggctaatttttgtatttttagtagagacggggtttcaccatggtggccaggctgggttctggttgtttatgatctttattttttggtgatctaggaaccaaacaacaagaaattgttgtttcccgtgggtaagaggatcc

SEQ ID NO: 7 - DNA, 1597 bp, Artificial sequence
  SF3B3int2 Minigene
aagcttggcaatccggtactgttggtaaatttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgtggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttgaattgtgtgaaatttcaaatattggattaggaaatacaaagttactgaaagtgaggtactaatgtttataaaataaaaactttttcttgccatttgcagatttaacatttttgagtcaatccaagtgccaccatgcaggaggttcatgattgtgtagagtaagacataattttgttgaggtttaactctgaatacttaatgtggtactgaatacttaatgtggtactgagaggcagcctaactgaccacacagcattcatatttcatgttgttattttctctgatcctcattatggctcttcctgcgggtttgagtcttcatttgggtaatttagttattctttgctgcatttgattaaggaaatacaattcctgacctcgtgatccacccaccttagcctcccaaagtgctgggattacaggcatgagctactgtgccctgctgaaagcacatttttaatactaattttatctttcaaattccttttccaattcagtcttcctttttatctaaaatgatggagaagttttcttttttttttttttttttttgagacgtagtcttgctgtgttgcccaggctggagtacagtagtgcgacctcggctcactgcaaactccgtctcccaggttcaagcgattctcctgtgtcagcctctggagtagctgggattataggcacatcttgctgcgtttcccaggatggagtgcaatggcaggattataacccactacattctctgaactcctgggttcaaactgtcctcccacgtagctgggactagaggcacaggccatcacacctgactaatttttttttgagacggagttttgctctgttgcccaggccggagtgcagtggcacagtcttggctcacagcaacctctacctcctgggttcaagcgattctcctgccttagcctcccgagtagctgggattacaggcatgtgccaccacgcctggctaatttttgtatttttagtagagacggggtttcaccatggtggccaggctgggttctggttgtttatgatctttattttttggtgatctaggaaccaaacaacaagaaattgttgtttcccgtgggtaagaggatcc

SEQ ID NO: 8 - DNA, 1469 bp, Artificial sequence
  SF3B3i3 Minigene
aagcttggcaatccggtactgttggtaaatttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgtggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttgaattgtgtgaaatttcaaatattggattaggaaatacaaagttactgaaagtgaggtactaatgtttataaaataaaaactttttcttgccatttgcagatttaacatttttgagtcaatccaagtgccaccatgcaggaggttcatgattgtgtagagtaagacataattttgttgaggtttaactctgaatacttaatgtggtactgaatacttaatgtggtactgagaggcagcctaactgaccacacagcattcatatttcatgttgttattttctctgatcctcattatggctcttcctgcgggtttgagtcttcatttgggtaatttagttattctttgctgcatttgattaaggaaatacgtgccaccacacctggctaatttttgtatttttagtagagatgggatcccaccatgttggccaggctggtttcaaactcctgacctcaagtgatctgcctgcctcagcctcccaaagtgctgggataacaggtgtgagccactgtgcgtggccttttttttttttttttttttttttagagacgatatcttgctgcgtttcccaggatggagtgcaatggcaggattataacccactacattctctgaactcctgggttcaaactgtcctcccacgtagctgggactagaggcacaggccatcacacctgactaatttttttttgagacggagttttgctctgttgcccaggccggagtgcagtggcacagtcttggctcacagcaacctctacctcctgggttcaagcgattctcctgccttagcctcccgagtagctgggattacaggcatgtgccaccacgcctggctaatttttgtatttttagtagagacggggtttcaccatggtggccaggctgggttctggttgtttatgatctttattttttggtgatctaggaaccaaacaacaagaaattgttgtttcccgtgggtaagaggatcc

SEQ ID NO: 9 - DNA, 1283 bp, Artificial sequence
  SF3B3i4 Minigene
aagcttggcaatccggtactgttggtaaatttctgtacaacttaaccttgcagagagccactggcatcagctttgccattcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgtggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttgaattgtgtgaaatttcaaatattggattaggaaatacaaagttactgaaagtgaggtactaatgtttataaaataaaaactttttcttgccatttgcagatttaacatttttgagtcaatccaagtgccaccatgcaggaggttcatgattgtgtagagtaagacataattttgttgaggtttaactctgaatacttaatgtggtactgaatacttaatgtggtactgagaggcagcctaactgaccacacagcattcatatttcatgttgttattttctctgatcctcattatggctcttcctgcgggtttgagtcttcatttgggtaatttagttattctttgctgcatttgattaaggaaatacatcttgctgcgtttcccaggatggagtgcaatggcaggattataacccactacattctctgaactcctgggttcaaactgtcctcccacgtagctgggactagaggcacaggccatcacacctgactaatttttttttgagacggagttttgctctgttgcccaggccggagtgcagtggcacagtcttggctcacagcaacctctacctcctgggttcaagcgattctcctgccttagcctcccgagtagctgggattacaggcatgtgccaccacgcctggctaatttttgtatttttagtagagacggggtttcaccatggtggccaggctgggttctggttgtttatgatctttattttttggtgatctaggaaccaaacaacaagaaattgttgtttcccgtgggtaagaggatcc

SEQ ID NO: 10 - DNA, 880 bp, Artificial sequence
  Benc1 Minigene
aagcttggcaatccggtactgttggtaaactttcctggactgtgtgcagcagttcaaagaagaggttgagaaaggcgagacacgtttttgtcttccctacaggtcagtatagcgcaatgctgagtgaacttgtataggaccaaattgaacagaattgaagcagcagtaataatggaggcaaaacagtataaatggtgggaaagcaaaaaaatggcacagaatcacatgacattgatgggcagagactgacccagtactgcaagcaggacagaatgtgaccacctagtccttggagtagaagaatgttgccttagatggtttagggggttttcttggcatagagctcaagcctcctctccagtttcaagaaggtttttccctgaagcccttggaaatacacaggaatgcttcttttgatttttctaggatctcattggccaccatggatggaaactctagtttttacttagagtaaggctgcttctatcaccccaaaacaaagcatttcttttcttctgcctcctttctcccccgccaagttcagggatgcctaggaaaacagcaagaagtactgtaaccctcactcacccctactccaggcttctcttctctttgggtgtgtgtcctgtccctgtactctgcaatgacaatgaggctcttgtgacagtcttcttgatttttggctttcaagcaaatccaaaatacactatcattcctcaccaagtgttctttaatagataaaatgatgtgaatggtcccatgatcaagtccctgccccgaacttacttaactcagatctcagttactaatgagttttgctctgtccatctgcaggatcgatgtggaaaaaaacaagattgaaaacacaataaccggatcc

SEQ ID NO: 11 - DNA, 1284 bp, Artificial sequence
  C12orf4 Minigene
aagcttggcaatccggtactgttggtaaaagtatgtcggcaagtcagctggtttcccagtgcaggacgcgctgacagctcgagtgagcggactgcccaggacttcggaactagacgaggtgagcccagaaccgagtgaggatgccctttcagactgacaaccgttacagagggattagtcccctcacccggctgaagtttctcagctcgaccgtctgtcttccttttccctgattaatgtgagcctcagagcctccgctctgttggctgtgtgtgttcggtccagaccttccatcccattttcttatgggaaaccttcctggatttcacggttgctaaaactgaacccaggcttcaggatagaccaataaaaatttgtattgctttacgtttaattttaattctttctgagtaaaacaaataccctatctctttgcattatggtgaaatagacgaagatttttgtgtatagcctacatacagatttgaaaatatagtctggataattcaacttagccccgccccacgacatgtacaaaagtcctttgagttttttttatatttcaagtgatatctttttaaataaccgtatgaataattttttgttctttatttttccagtgagcaccataaaataaaaacgccatacaatccaacaattatttattagttcttgccattcgcaacatcctgcctattgccaccatggaatacaagacagtattccttccacttcaagaagactgttttctagccaagagtaagaaatagagcgtgataagtgccatgattcaaataagtctaggctaatatgagagcctgtcgggacccaatccagttttagggagttggataagtcttcctagaggcttaactgagccctgaagaattagcaattgttatccaatgaaattaagatcagtaagagtgctctaggcaaaggaaaaggcattcaaaaagattcggcatggaaggagaagtttggtgttgacagaagttcagaatggctgttttcttaaatcaagtttaaaacaaagtgtctttttttttaaagaagcactttgttttcatcatgaagatggaagtacagggataaacacttgggaaaaactagaataattggctcatagacaaaatctcttgaatggtggaatgttttcttagaaatgggacagcaataatgaaacataatttctttatggacttataacctactgcttttttgttagttctgtacaaatcctgtcaaaaaatatcaaaaaaaacagagaaagtaaccggatcc

SEQ ID NO: 12 - DNA, 985 bp, Artificial sequence
  PDXDC2 Minigene
aagcttggcaatccggtactgttggtaaactcggcttgcccttcccctgcttgtgccgtgtaccctgtaacactgtgtttcgatcccagcatcagatggtgagttctacttttggtttgtaaaatcatgtgggattgtgtttggaaactgcctttcaagtgaagttctttttgctggcctccagataattagtaacttactgatgtattgcttggacccttgcattgtgtaatatatgcttctgttaatatgtgaatgtccatgaaggttggttggtatataggtcaggagcacctggcttttaaggagtcttgtaattactgtatcgcctgctttaagtagaaacatggcagctcaattaagcaccagaaatttcttctgaagctcctacttttaatgagttagcactgttgcctggatttatgaatgggcagattttgaaaatgaggttcttcattgtcatcgtggaattgagttctgcttaatttcagacatgcaggtgataaatctctccttaatgattccagaaggaaaaggaagggatggattttagagttttttatttcttttgtcaaggtagttttattttctagttcaggtacatcagtgagtaatagtagagatacattcatggctgttttaggagagttaagagaacatttcatttaaatgtaaatgtttaaaatcaaagaaatgttcctgcatatgtacagtttttacctttaatcttgtttaactgatttttctagtcttcaaggaaaactatttgattttcacatctatgatgagagaaagccaccatgaaaaattgtcaagagtaagaattgatttgacattacttttgagagttatctgcttcttttgcaagtcataatttttcattttatagcttttatactaggcacctcattttttaatgatttgttttggttccaggatcttgccttcctggaaaaacttattaaaattgatatataacggatcc

SEQ ID NO: 13 - DNA, 1158 bp, Artificial sequence
  SF3B3.OPTXonNGS Minigene
aagcttggcaatccggtactgttggtaaatttctgtacaacttaaccttgcagagagccactggcatcagctttgccatcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgtggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttgaattgtgtaaatttcaaatattggattaggaaatacaaagttactgaaagtgaggtactaatgtttataaaataaaaactttttcttgccatttgcagatttaacatttttgagtcaatccaagtaatgcaggagggccaccatgggtgtaccagagtaagacataattttgttgaggtttaactctgaatacttatgtggtactgaatacttaatgtggtactgagaggcagcctaactgaccacacagcattcatatttcatgttgttattttctctgatcctcattatggctcttcctgcgggtttgagtcttcatttgggtaatttagttattctttgctgcatttgattaaggaaatacgtatatgggaatttggagatttgtgactgacttcactatgtctaatttttttttgagacggagttttgctctgttgcccaggccggagtgcagtggcacagtcttggctcacagcaacctctacctcctgggttcaagcgattctcctgccttagcctcccgagtagctgggattacaggcatgtgccaccacgcctggctaatttttgtatttttagtagagacggggtttcaccatggtggccaggctgggttctggttgtttatgatctttatttttggtgatctaggaacggatcc

SEQ ID NO: 14 - DNA, 1158 bp, Artificial sequence
  SF3B3.OPTXonKGS Minigene
aagcttggcaatccggtactgttggtaaatttctgtacaacttaaccttgcagagagccactggcatcagctttgccatcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgtggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttgaattgtgtaaatttcaaatattggattaggaaatacaaagttactgaaagtgaggtactaatgtttataaaataaaaactttttcttgccatttgcagatttaacatttttgagtcaatccaagtaatgcaggagggccaccatgggtgtaccagagtaagacataattttgttgaggtttaactctgaatacttatgtggtactgaatacttaatgtggtactgagaggcagcctaactgaccacacagcattcatatttcatgttgttattttctctgatcctcattatggctcttcctgcgggtttgagtcttcatttgggtaatttagttattctttgctgcatttgattaaggaaatacgtatatgggaatttggagatttgtgactgacttcactatgtctaatttttttttgagacggagttttgctctgttgcccaggccggagtgcagtggcacagtcttggctcacagcaacctctacctcctgggttcaagcgattctcctgccttagcctcccgagtagctgggattacaggcatgtgccaccacgcctggctaatttttgtatttttagtagagacggggtttcaccatggtggccaggctgggttctggttgtttatgatctttatttttggtgatctaggaaaggatcc

SEQ ID NO: 15 - DNA, 1158 bp, Artificial sequence
  SF3B3.OPTXonRGS Minigene
aagcttggcaatccggtactgttggtaaatttctgtacaacttaaccttgcagagagccactggcatcagctttgccatcttggaaacttttctggtaagttctctcgttaccatcttttgaaattttaagtgaattaatacatatcttgcttagtctcttgtgcaggaaatgttttccatttatgacaaaacagttgttggtaaatgtaaacaattcaaattatgagatgaggtgttaagttctggtgcattatctgttacctatttcagatgcatttcctagttcacaaattgtgtaatgattcttgtcagggcacacttttcttggctgcttacctagtgtggtttggtgcttgaagagatagaacttttgtatgagagaaaagggaaaatgcagtttaggtagggagtgctgtctgcatagaaacagataatttgcttacgtttaccatgtggggaatatttttgtaaagatggattaaggctaggtttgaattgtgtaaatttcaaatattggattaggaaatacaaagttactgaaagtgaggtactaatgtttataaaataaaaactttttcttgccatttgcagatttaacatttttgagtcaatccaagtaatgcaggagggccaccatgggtgtaccagagtaagacataattttgttgaggtttaactctgaatacttatgtggtactgaatacttaatgtggtactgagaggcagcctaactgaccacacagcattcatatttcatgttgttattttctctgatcctcattatggctcttcctgcgggtttgagtcttcatttgggtaatttagttattctttgctgcatttgattaaggaaatacgtatatgggaatttggagatttgtgactgacttcactatgtctaatttttttttgagacggagttttgctctgttgcccaggccggagtgcagtggcacagtcttggctcacagcaacctctacctcctgggttcaagcgattctcctgccttagcctcccgagtagctgggattacaggcatgtgccaccacgcctggctaatttttgtatttttagtagagacggggtttcaccatggtggccaggctgggttctggttgtttatgatctttatttttggtgatctaggcgcggatcc

SEQ ID NO: 16 - DNA, 195 bp, Artificial sequence
  SF3B3 Minigene sequence of FIG. 3F
agcttggcaatccggtactgttggtaaatttctgtacaacttaaccttgcagagagccactggcatcagctttgccattcttggaaacttttctggaaccaaacaacaagaaattgttgtttcccgtgggtaagaggatccgaagatgccaaaaacattaagaagggcccagcgccattctacccactcgaagac

SEQ ID NO: 17 - protein, 64 aa, Artificial sequence
  Amino acids encoded by SF3B3 Minigene sequence of FIG. 3F
SLAIRYCWISVQLNLAESHWHQLCHSWKLFWNQTTRNCCFPWVRGSEDAKNIKKGPAPFYPLED

SEQ ID NO: 18 - DNA, 203 bp, Artificial sequence
  BENC1 Minigene sequence of FIG. 3F
aagcttggcaatccggtactgttggtaaactttcctggactgtgtgcagcagttcaaagaagaggttgagaaaggcgagacacgtttttgtcttccctacaggatcgatgtggaaaaaaacaagattgaaaacacaataaccggatccgaagatgccaaaaacattaagaagggcccagcgccattctacccactcgaagacg

SEQ ID NO: 19 - protein, 66 aa, Artificial sequence
  Amino acids encoded by BENC1 Minigene sequence of FIG. 3F
SLAIRYCWTFLDCVQQFKEEVEKGETRFCLPYRIDVEKNKIENTITGSEDAKNIKKGPAPFYPLED

SEQ ID NO: 20 - DNA, 199 bp, Artificial sequence
  PDXDC2 Minigene sequence of FIG. 3F
aagcttggcaatccggtactgttggtaaactcggcttgcccttcccctgcttgtgccgtgtaccctgtaacactgtgtttcgatcccagcatcagatggatcttgccttcctggaaaaacttattaaaattgatatataacggagaagatgccaaaaacattaagaagggcccagcgccattctacccactcgaagacg

SEQ ID NO: 21 - protein, 66 aa, Artificial sequence
  Amino acids encoded by PDXDC2 Minigene sequence of FIG. 3F
KLGNPVLLVNSACPSPACAVYPVTLCFDPSIRWILPSWKNLLKLIYNGEDAKNIKKGPAPFYPLED

SEQ ID NO: 22 - DNA, 230 bp, Artificial sequence
  C12ORF4 Minigene sequence of FIG. 3F
aaagcttggcaatccggtactgttggtaaaagtatgtcggcaagtcagctggtttcccagtgcaggacgcgctgacagctcgagtgagcggactgcccaggacttcggaactagacgagttctgtacaaatcctgtcaaaaaatatcaaaaaaaacagagaaagtaaccggatccgaagatgccaaaaacattaagaagggcccagcgccattctacccactcgaagacg

SEQ ID NO: 23 - protein, 76 aa, Artificial sequence
  Amino acids encoded by C12ORF4 Minigene sequence of FIG. 3F
KLGNPVLLVKVCRQVSWFPSAGRADSSSERTAQDFGTRRVLYKSCQKISKKTEKVTGSEDAKNIKKGPAPFYPLED

SEQ ID NO: 24 - DNA, 10 bp, Artificial Sequence
  LMI070 Binding sequence in SF3B3
agagtccgac

SEQ ID NO: 25 - DNA, 10 bp, Artificial Sequence
  LMI070 Binding sequence in BENC1
agagtaaggc

SEQ ID NO: 26 - DNA, 10 bp, Artificial Sequence
  LMI070 Binding sequence in GXYLT1
agagtatagt

SEQ ID NO: 27 - DNA, 10 bp, Artificial Sequence
  LMI070 Binding sequence in C12orf4, PDXDC2, and CIP2A
agagtaagaa

SEQ ID NO: 28 - DNA, 10 bp, Artificial Sequence
  LMI070 Binding sequence in RARS
agagtaggat

SEQ ID NO: 29 - DNA, 10 bp, Artificial Sequence
  LMI070 Binding sequence in WNK1
agagtaggtg

SEQ ID NO: 30 - DNA, 10 bp, Artificial Sequence
  LMI070 Binding sequence in WDR27
agagtaagca

SEQ ID NO: 31 - DNA, 10 bp, Artificial Sequence
  LMI070 Binding sequence in ITF57
agagtaggcc